SEQUENCE LISTING (32 sequences)

SEQ ID NO: 1 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 2 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 3 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 4 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 5 - protein, 214 aa, synthetic construct
DIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 528 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 7 - protein, 8 aa, Murine Leukemia Virus
KSPWFTTL

SEQ ID NO: 8 - protein, 214 aa, synthetic construct
DIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, synthetic construct
DIQMTQSPSSLSASVGDRVTITCKSSEKLWANVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 214 aa, synthetic construct
DIQMTQSPSSLSASVGDRVTITCKAREKLWSAVAWYQQKPGKAPKLLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, synthetic construct
DIQMTQSPSSLSASVGDRVTITCKASEKLWSNVGWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, synthetic construct
DIQMTQSPSSLSASVGDRVTITCKASEKLWSNVGWYQQKPGKAPKLLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 538 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSSGGPALFKSSFPPGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 14 - protein, 518 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSSGGPALFKSSFPPGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 15 - protein, 508 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSSGGPALFKSSFPPGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 16 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSSGGPALFKSSFPPGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSSGGPALFKSSFPPGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 17 - protein, 518 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSSGGPALFKSSFPPGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSSGGPALFKSSFPPGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 18 - protein, 518 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSSGGPALFKSSFPPGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSSGGPALFKSSFPPGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 19 - protein, 518 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSSGGPALFKSSFPPGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 20 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPHLFKSFPFPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPHLFKSFPFPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 21 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPHKFKSKFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPHKFKSKFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 22 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPKLLFHLFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPKLLFHLFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 23 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPKKLFHIFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPKKLFHIFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 24 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSFPPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSFPPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 25 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSLPPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSLPPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 26 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSFPFPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSFPFPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 27 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPAKFRHLFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPAKFRHLFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 28 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALLKSIFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALLKSIFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 29 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPAKLRSKFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPAKLRSKFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 30 - protein, 534 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPLYAKLKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPLYAKLKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 31 - protein, 534 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPLAQKVKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPLAQKVKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 32 - protein, 534 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPLAQKLRSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPLAQKLRSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

(19) United States
(12) Patent Application Publication
(10) Pub. No.: US 2024/0374687 A1
(43) Pub. Date: Nov. 14, 2024

(54) IL-2 PRODRUG

(71) Applicant: Werewolf Therapeutics, Inc., Watertown, MA (US)

(72) Inventors: Jose Andres SALMERON-GARCIA, Acton, MA (US); William WINSTON, West Newton, MA (US); Daniel HICKLIN, Boston, MA (US); Cynthia SEIDEL-DUGAN, Belmont, MA (US); Heather BRODKIN, West Newton, MA (US); Christopher NIRSCHL, Medfield, MA (US); Tiziana CAFARELLI, Watertown, MA (US)

(21) Appl. No.: 18/626,430

(22) Filed: Apr. 4, 2024

Related U.S. Application Data
(63) Continuation of application No. PCT/US2022/077772, filed on Oct. 7, 2022.
(60) Provisional application No. 63/253,964, filed on Oct. 8, 2021.
(60) Provisional application No. 63/290,941, filed on Dec. 17, 2021.
(60) Provisional application No. 63/328,524, filed on Apr. 7, 2022.

(51) Int. Cl. A61K 38/20; A61P 35/00

(52) U.S. Cl. CPC A61K 38/2013; A61P 35/00

(57) ABSTRACT

This disclosure relates to methods and compositions for treating cancer using an inducible IL-2 prodrug.

The present application is a continuation of International Application No. PCT/US2022/077772, filed on Oct. 7, 2022, which designated the United States, which claims the benefit of U.S. Provisional Application No. 63/253,964, filed on Oct. 8, 2021, U.S. Provisional Application No. 63/290,941, filed on Dec. 17, 2021, and U.S. Provisional Application No. 63/328,524 filed on Apr. 7, 2022, the entire contents of each of which are incorporated herein by reference in their entireties.

This application contains a Sequence Listing which has been submitted electronically in XML format. The Sequence Listing XML is incorporated herein by reference. Said XML filed, created on Oct. 7, 2022, is named 761146.320320_SL.xml and is 42,925 bytes in size.

### 1. BACKGROUND

Cancer immunotherapy has rapidly established itself as the fourth pillar of cancer treatment largely owing to the clinical success of checkpoint inhibitors (1-3). Despite the durable responses achieved by some patients using these new therapies, the proportion of responders is still relatively low and restricted to only some cancer types. Tumor mutational burden, the presence or absence of T cell infiltration in tumors, and the overall immunosuppressive microenvironment of tumors greatly influences the response to immunotherapies. Although immune checkpoint blockade can prevent the physiological stop-signal that arises in response to immune activation, other approaches can be used to positively stimulate the anti-tumor immune response. One approach involves the use of immune-activating cytokines. Numerous preclinical and clinical studies have demonstrated the promise of cytokine therapy to increase anti-tumor immunity. In fact, these were some of the first cancer immunotherapies approved for clinical use. However, systemic toxicity and poor pharmacokinetic profiles have limited their clinical application (4).

Interleukin (IL)-2 is a critical cytokine driving the immune-mediated killing of cancer cells, and whose mechanism of action includes stimulation of both innate and adaptive immune cells. The IL-2 receptor (IL-2R) is composed of three subunits: cluster of differentiation (CD)25 (IL-2Rγ), CD122 (IL-2Rγ), and CD132 (IL-2Rγ). Signal transduction is mediated through a heterodimer of CD122 and CD132. Together, these molecules form the IL-2 medium-affinity receptor, which is expressed on natural killer (NK) cells, monocytes, macrophages, and resting CD4+ and CD8+ T cells. The trimeric IL-2 high-affinity receptor (CD25/CD122/CD132) is present on activated T and NK cells and constitutively expressed on CD4+FoxP3+ regulatory T cells (Tregs). IL-2 increases the proliferation and activation of T cells and NK cells, and induces the differentiation of CD8+ T cells into effector and memory cells (5,6). Recombinant human IL-2 (proleukin) is approved for clinical use in metastatic melanoma and renal cell carcinoma as a high-dose therapy, but this treatment is associated with serious side effects, including vascular leakage syndrome and hypotension, limiting its practical use (5,7).

To address the limitations of IL-2 high-dose therapy, several approaches have been pursued to develop next-generation IL-2 molecules that only bind the medium-affinity receptor (CD122/CD132) in the hope of alleviating toxicities and reducing the activation of Tregs (7-10). However, many of these molecules still activate IL-2 receptors on non-tumor specific immune cells located in normal tissues and therefore, may not minimize toxicities associated with IL-2 signaling. Molecules that block IL-2 signaling in the periphery while delivering a fully active native IL-2 in the tumor microenvironment may be a more appropriate approach to achieve the full potential of IL-2 anti-tumor activity with minimal systemic toxicities.

Inducible forms of IL-2, that are conditionally activated in the tumor microenvironment through protease cleavage to release the fully active, native IL-2 cytokine within the tumor to stimulate a potent anti-tumor immune response, are described in WO2021097376. These IL-2 prodrugs include a native IL-2 molecule attached through a protease cleavable linker to a half-life extension domain (e.g., anti-human serum albumin antibody binding fragment such as a VH domain) and an IL-2 blocking element (e.g., anti-IL-2 antibody binding fragment, such as a Fab) to block binding of IL-2 to IL-2β/γ receptors on normal tissue in the periphery. Upon cleavage of the protease cleavable linker, fully active native IL-2 is released within the tumor to stimulate a potent anti-tumor immune response.

### 2. SUMMARY

This disclosure relates to compositions and methods for treating cancer using an inducible IL-2 prodrug. The method generally comprises administering to a subject in need thereof an effective amount of an inducible IL-2 prodrug. The inducible IL-2 prodrug can be Compound 1, Compound 2, Compound 3, or Compound 4. The inducible IL-2 prodrug can be any one of Compounds 5-29.

The inducible IL-2 prodrug is conditionally active. The inducible IL-2 prodrug comprises two polypeptide chains. The first polypeptide chain can comprise from amino to carboxy terminus: the IL-2 polypeptide—a protease cleavable linker—an anti-human serum albumin (HSA) binding single antibody variable domain—a linker that is preferably protease cleavable—VH and CH1 of an antibody that binds IL-2. The first polypeptide chain can comprise from amino to carboxy terminus: the IL-2 polypeptide—a protease cleavable linker—VH and CH1 of an antibody that binds IL-2—a linker that is preferably protease cleavable—an anti-human serum albumin (HSA) binding single antibody variable domain. The second polypeptide chain comprises a VL and CL of an antibody that binds IL-2 and that together with the VH and CH1 of the first polypeptide chain form a Fab that binds the IL-2 polypeptide. When the inducible IL-2 prodrug is not in a site of interest (e.g., a tumor microenvironment), the prodrug typically remains intact. The intact prodrug has attenuated IL-2 receptor agonist activity. When the inducible IL-2 prodrug is in a site of interest (such as a tumor microenvironment), the protease cleavable linker is cleaved by a protease active in the site of interest, releasing an unattenuated form of IL-2. This conditional activity preserves the immune stimulatory effects of IL-2 while limiting the systemic toxicity associated with non-inducible IL-2 therapy. The intact IL-2 prodrug contains an element that extends its half-life, but the post-cleavage unattenuated form of IL-2 does not. As a result, the short half-life of IL-2 effectively limits toxicity outside of the site of interest.

As further described and exemplified herein, following systemic administration the amount of inducible IL-2 prodrug in the circulation (plasma) can be at least about 5-fold greater than the amount in the tumor, e.g., the amount in the circulation can be about at least about 5-fold, at least about 10-fold, at least about 15-fold, at least about 18-fold, at least about 20-fold, or at least about 25-fold greater than the amount of inducible IL-2 prodrug in the tumor. While more prodrug is found in the circulation than in the tumor microenvironment, the prodrug is processed (cleaved) to a greater extent in the tumor microenvironment to release active IL-2. Following systemic administration, there can be at least about 40-fold more cleavage of the prodrug to release active IL-2 in the tumor microenvironment compared to the circulation. In embodiments, there can be at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 93-fold, at least about 95-fold, or at least about 100-fold more cleavage of the inducible IL-2 prodrug in the tumor microenvironment compared with the circulation.

This disclosure relates to a method for treating cancer, comprising administering to a subject in need thereof an effective amount of an inducible interleukin-2 (IL-2) prodrug, wherein the inducible IL-2 prodrug is administered systemically, is activated by cleavage by a protease that has higher activity in the tumor microenvironment than in other locations, and results in at least about 40-fold more cleavage of the inducible IL-2 prodrug in the tumor microenvironment compared with the circulation. The method can result in a significant increase in the tumor reactive CD8+/Treg ratio.

This disclosure relates to a method for inducing immunological memory to a tumor. The method comprises administering to a subject in need thereof and effective amount of an inducible interleukin-2 (IL-2) prodrug, wherein the inducible IL-2 prodrug is administered systemically, is activated by cleavage by a protease that has higher activity in the tumor microenvironment than in other locations. Following systemic administration the amount of inducible IL-2 prodrug in the circulation (plasma) can be at least about 5-fold greater that the amount in the tumor, e.g., the amount in the circulation can be about at least about 5-fold, at least about 10-fold, at least about 15-fold, at least about 18-fold, at least about 20-fold, or at least about 25-fold greater than the amount of inducible IL-2 prodrug in the tumor. Following systemic administration, there can be at least about 40-fold more cleavage of the prodrug to release active IL-2 in the tumor microenvironment compared to the circulation. In embodiments, there can be at least about 45-fold, at least about 50-fold, at least about 55-fold, at least about 60-fold, at least about 65-fold, at least about 70-fold, at least about 75-fold, a t least about 80-fold, at least about 85-fold, at least about 90-fold, at least about 93-fold, at least about 95-fold, or at least about 100-fold more cleavage of the inducible IL-2 prodrug in the tumor microenvironment compared with the circulation. The immunological memory can be characterized by the presence of tumor reactive CD8+ cells with a memory phenotype (e.g., CD8+CD44hiCD62low), by tumor reactive CD8+ cells that produce TNF, IFNgamma and/or granzyme B upon restimulation, or tumor reactive CD8+ cells with a memory phenotype that produce TNF, IFNgamma and/or granzyme B upon restimulation.

This disclosure relates to a method for selectively activating effector CD8+ T cells in the tumor microenvironment, and to a method for selectively activating tumor infiltrating lymphocytes. These methods comprising administering to a subject in need thereof and effective amount of an inducible interleukin-2 (IL-2) prodrug, wherein the inducible IL-2 prodrug is administered systemically, is activated by cleavage by a protease that has higher activity in the tumor microenvironment than in other locations, and results a significantly higher frequency of CD8+ T cells that produce TNF and/or IFNgamma within the tumor in comparison to peripheral tissue. cleavage by a protease that has higher activity in the tumor microenvironment than in other locations. Following systemic administration the amount of inducible IL-2 prodrug in the circulation (plasma) can be at least about 5-fold greater that the amount in the tumor, e.g., the amount in the circulation can be about at least about 5-fold, at least about 10-fold, at least about 15-fold, at least about 18-fold, at least about 20-fold, or at least about 25-fold greater than the amount of inducible IL-2 prodrug in the tumor. Following systemic administration, there can be at least about 40-fold more cleavage of the prodrug to release active IL-2 in the tumor microenvironment compared to the circulation. In embodiments, there can be at least about 45-fold, at least about 50-fold, at least about 55-fold, at least about 60-fold, at least about 65-fold, at least about 70-fold, at least about 75-fold, a t least about 80-fold, at least about 85-fold, at least about 90-fold, at least about 93-fold, at least about 95-fold, or at least about 100-fold more cleavage of the inducible IL-2 prodrug in the tumor microenvironment compared with the circulation. These methods can result in a significant increase in the tumor reactive CD8+/Treg ratio in the tumor microenvironment.

In embodiments of the methods of this disclosure the inducible IL-2 prodrug can be administered about twice a week or less frequently, once a week or less frequently or about once every two weeks or less frequently. In certain embodiments, the inducible IL-2 prodrug can be administered about once every two weeks.

Preferred, inducible IL-2 prodrugs for use in the methods of this disclosure are Compound 1, Compound 2, Compound 3, Compound 4 or an amino acid sequence variant of any of the foregoing. Other preferred inducible IL-2 prodrugs for use in the methods of this disclosure are Compounds 5-29. Compound 1 comprises a first polypeptide chain of SEQ ID NO:1 and a second polypeptide chain of SEQ ID NO:5, and the amino acid sequence variant of Compound 1 can comprise a first polypeptide chain that has at least about 80% identity to SEQ ID NO:1 and a second polypeptide chain can comprise at least about 80% identity to SEQ ID NO:5. Compound 2 comprises a first polypeptide chain of SEQ ID NO:2 and a second polypeptide chain of SEQ ID NO:5, and the amino acid sequence variant of Compound 2 can comprise a first polypeptide chain that has at least about 80% identity to SEQ ID NO:2 and a second polypeptide chain that has at least about 80% identity to SEQ ID NO:5. Compound 3 comprises a first polypeptide chain of SEQ ID NO:3 and a second polypeptide chain of SEQ ID NO:5, and the amino acid sequence variant of Compound 3 can comprise a first polypeptide chain that has at least about 80% identity to SEQ ID NO:3 and a second polypeptide chain that has at least about 80% identity to SEQ ID NO:5. Compound 4 comprises a first polypeptide chain of SEQ ID NO:1 and a second polypeptide chain of SEQ ID NO:4, and the amino acid sequence variant of Compound 4 can comprise a first polypeptide chain that has at least about 80% identity to SEQ ID NO:4 and a second polypeptide chain that has at least about 80% identity to SEQ ID NO: 5.

### 3. BRIEF DESCRIPTION OF THE DRAWINGS

FIGS. 1A-1F show the design and development of inducible IL-2 prodrugs represented by Compound 1. FIG. 1A is a diagram of the components of Compound 1. FIG. 1B depicts a non-reduced SDS-PAGE comparing intact and protease-cleaved Compound 1 (IL-2, anti-HSA half-life extension domain, and the Fab inactivation domain). FIG. 1C shows the in vitro activity of Compound 1 in the HEK-Blue IL-2 reporter assay comparing intact (squares), and protease-activated (cleaved) Compound 1 (triangles) to rhIL-2 (circles). FIG. 1D shows the in vitro activity of intact (squares) and cleaved (triangles) Compound 1 in primary human Tblasts compared to rhIL-2 (circles). FIG. 1E shows the in vitro activity of intact (squares) and cleaved (triangles) Compound 1 in primary murine Tblasts compared to rhIL-2 (circles). FIG. 1F shows the in vitro activity of intact (circles) and cleaved (triangles) Compound 1-NC in primary human Tblasts compared with rhIL-2 squares). FIGS. 1C-1F curves are representative of at least duplicate wells and depict the mean±SD for individual points; data are representative of at least two experiments.

FIGS. 2A-2J depict that Compound 1 induced tumor regression in a cleavage-dependent manner. FIG. 2A is a series of graphs that show tumor volume over time in mice treated with various doses of Compound 1, Compound-NC (non-cleavable control), or vehicle. Spider plots for individual mice are shown (dashed lines), and the average tumor volume for the group is shown as the bold line. FIG. 2B are graphs that show the body weight and survival from individual mice over time is shown treated with either Compound 1 or WWO177 (a Compound 1 variant lacking the inactivation domain). Body weight and survival from individual mice over time is shown. Dosing of WWO177 was halted after 2 doses due to excessive toxicity, whereas mice receiving Compound 1 were given all four doses. FIG. 2C shows a western blot for Compound 1 that was diluted in murine plasma from either wild-type or MC38 tumor-bearing mice and incubated at 37° C. for 24, 48, or 72 hours before Compound 1 processing. Intact and cleaved controls were prepared in vitro. Data are representative of n=3 mice. FIG. 2D are graphs showing the tumor volume over time in mice treated with efficacious amounts of either Compound 1 (5.04 μM total) or rhIL-2 (15.5 μM total). Spider plots for individual mice are shown. FIG. 2E is a graph showing total IL-2 over time in the plasma from tumor-bearing mice. Samples were taken at various timepoints and analyzed for either the presence of the total inducible IL-2 protein using an ELISA that detects both intact Compound 1 as well as free IL-2. FIG. 2F is a graph showing total IL-2 over time in tumor samples from tumor-bearing mice. Samples were taken at various timepoints and analyzed for either the presence of the total inducible IL-2 protein using an ELISA that detects both intact Compound 1 as well as free IL-2. FIG. 2G is a graph showing total IL-2 over time in the plasma from tumor-bearing mice. Samples were taken at various timepoints and analyzed for either the presence of the total inducible IL-2 protein using an ELISA that detects free human IL-2 using an AlphaLISA specific for unblocked human IL-2. Mice received only two doses, and the timing of the doses is indicated by the arrows on the dependent (x) axis. FIG. 2H a graph showing total IL-2 over time in tumor samples from tumor-bearing mice. Samples were taken at various timepoints and analyzed for either the presence of the total inducible IL-2 protein using an ELISA that detects free human IL-2 using an AlphaLISA specific for unblocked human IL-2. Mice received only two doses, and the timing of the doses is indicated by the arrows on the dependent (x) axis. FIGS. 2E-2H are presented as the mean±SD, and area under the curve measurements were calculated using GraphPad Prism software. FIG. 2I is a graph showing tumor volume (mm³) at day 18 with vehicle, Compound 1 at 25 μg, 50 μg, 100 μg and 300 μg, and Compound 1-NC at 300 μg. FIG. 2J is a graph showing that the anti-tumor activity of Compound 1 was greatly reduced in mice when CD8+ T cells were depleted by anti-CD8 antibody treatment twice per week.

FIG. 3A-3I demonstrate that Compound 1 induced anti-tumor memory response. FIGS. 3A-3B show the frequency of tetramer-positive CD8+ T cells in splenocytes. FIG. 3C-3D show the expression of the memory cell markers CD62L and CD44 on tetramer-positive CD8+ T cells in splenocytes. FIG. 3E-3F show the frequency of tetramer-positive CD8+ T cells producing TNF or IFNγ. FIG. 3G are pie graphs showing the analysis of polyfunctional tetramer-positive CD8+ T cells co-expressing IFNγ and TNF. FIG. 3H is a schematic of a tumor challenge and rechallenge study. Naïve mice or mice that had previously rejected MC38 tumors after IL-2 INDUKINE™ protein treatment were re-challenged with MC38 tumor cells 60 days following the initial implantation. No treatment was administered to these mice during the re-challenge.

FIG. 31 shows a graph depicting tumor volume from MC38 CR (n=15) or naïve (n=33) mice was measured over time. Data are presented as mean±SD, with P values derived from t tests (*, P<0.05; **, P<0.01; ***, P<0.001; ****, P<0.0001).

FIGS. 4A-4M show that treatment with Compound 1 increased immune cell activation and infiltration of MC38 tumors. FIG. 4A depicts a heatmap of transcripts with statistically significant differences in expression between the two treatments (Compound 1 and Vehicle control). Transcripts were excluded from the heat map if they had average normalized counts below 50. Each lane represents an individual animal. FIG. 4B is a plot of transcripts differentially expressed between Compound 1 and vehicle-treated mice. FIG. 4C depict specific pathway scores for Compound 1 or vehicle-treated mice. P values are derived from a 2-way ANOVA with multiple comparisons (***, P<0.001; ****, P<0.0001). FIG. 4D shows normalized gene counts from selected immune checkpoint genes. FIG. 4E depicts diagrams from flow cytometry analysis of TIL density of various immune populations, including fold change information between the vehicle- and Compound 1-treated groups. FIG. 4F show the ratio of total CD8+ T cells or tetramer-positive CD8+ T cells to Tregs within the TILs, including fold change information between the vehicle- and Compound 1-treated groups. FIGS. 4G-4H show the frequency of tetramer-positive CD8+ T cells producing IFNγ after re-stimulation with PMA/lonomycin. FIG. 41 shows pie graphs of the analysis of polyfunctional tetramer-positive CD8+ T cells by examining co-expression of IFNγ, TNF, and granzyme B after PMA/lonomycin restimulation. FIGS. 4J-4K show the frequency of tumor-infiltrating FoxP3+ Tregs producing IFNγ in the vehicle (control) and Compound 1 groups. FIGS. 4L-4M show the frequency of tumor-infiltrating FoxP3+ Tregs producing TNF after PMA/lonomycin restimulation in the vehicle (control) and Compound 1 groups. Unless otherwise stated, data are presented as the mean±SD, and P values are derived from t tests (*, P<0.05; **, P<0.01; ***, P<0.001; ****, P<0.0001).

FIGS. 5A-5C show that systemic treatment with Compound 1 preferentially activated tumor-infiltrating T cells. FIG. 5A are graphs showing the frequency of tetramer-negative CD8+ T cells in vehicle (control) and Compound 1 groups in the TILs, spleenocytes, DLN, and peripheral blood. FIG. 5B are graphs showing the frequency of CD4+ non-Tregs producing IFNγ in vehicle (control) and Compound 1 groups in TILs, spleenocytes, DLN, and peripheral blood after re-stimulation with PMA/lonomycin. FIG. 5C is a graph showing tumor volume over time in mice treated with either vehicle (n=10), Compound 1 alone (n=10), or Compound 1 with daily FTY720 (n=10) treatment. FTY720 dosing was initiated 24 hours prior to starting Compound 1 treatment (25 μg dose) and maintained daily (10 μg dose) throughout the experiment. Tumor volume (mean±SEM) was measured over time. Unless otherwise stated, data are presented as the mean±SD, and P values are derived from t tests (*, P<0.05; **, P<0.01; ***, P<0.001; ****, P<0.0001).

FIGS. 6A-6G show that treatment with Compound 1 increased CD8+ T cell activation and Treg fragility in B16-F10. FIG. 6A shows tumor volume measured over time in mice treated with vehicle, Compound 1 at 100 μg/animal and 200 μg/animal, and Compound 1 at 100 μg/animal and 200 μg/animal in combination with an anti-PD1 inhibitor. Data from individual mice (dashed lines) are depicted with the group average presented in the bold line. FIG. 6B depicts a heatmap of transcripts with statistically significant differences in expression between the two treatments (vehicle control and Compound 1). Transcripts were excluded from the heat map if they had average normalized counts below 50. Each lane represents an individual animal. FIGS. 6C-6H are a series of graphs showing the results of TILs that were re-stimulated and examined for production of effector cytokines and proteins and proliferation. FIG. 6C are representative flow plots of tetramer-positive CD8+ T cells. FIG. 6D are graphs showing the quantitative analysis from individual mice. FIG. 6E are representative flow plots of NK cells and FIG. 6F are graphs showing the quantitative analysis from individual mice. FIG. 6G are representative flow plots of FoxP3+ Tregs and FIG. 6H are graphs showing the quantitative analysis from individual mice. P values are derived from t tests (*, P<0.05; **, P<0.01; ***, P<0.001; ****, P<0.0001).

FIGS. 7A-7B show that Compound 1 was stable in human serum and selectively processed by human tumor cells. FIG. 7A shows the results from a western blot analysis where Compound 1 was diluted into healthy human serum from n=3 donors and incubated at 37° C. for 24 or 72 hours before Compound 1 processing was measured by western blot analysis for IL-2. FIG. 7B shows a graph of total activation when Compound 1 was exposed to primary cells and ex vivo inducible IL-2 protein cleavage was measured in primary human tumor samples (n=97) and primary human healthy cells (n=13).

FIGS. 8A-8D show activity of Compound 1 in additional human donors and mice. FIGS. 8A-8B show CD25 expression by Tblasts over time in response to PHA stimulation. Data in FIG. 8A are representative of an individual human donor. FIG. 8B is pooled data from n=3 donors. FIGS. 8C-8D are graphs showing in vitro activity of Compound 1 in primary human Tblasts (FIG. 8C) and murine Tblasts (FIG. 8D) derived from additional donors, comparing intact (circle) and protease-activated (cleaved) WTX-124 (downward triangle) with rhIL-2 (upward triangle).

FIGS. 9A-9B shows that Compound 1 is well tolerated in mice. FIG. 9A is a graph showing the body weight over time of mice that were implanted with MC38 tumor cells and allowed to grow to an average volume of 100-150 mm³ before mice were randomized into treatment groups. Labels in the legend represent the dose per mouse per dosing day. Mice were dosed IP twice a week for a total of four doses, and body weight was measured over time. Average body weight of n=12 mice per group is shown. FIG. 9B is a graph showing a representative therapeutic window of rhIL-2 and Compound 1 in MC38 tumor-bearing mice.

FIGS. 10A-10B shows that effector cytokine production of MC38 tumor-infiltrating tetramer-positive CD8+ T cells. MC38 tumor cells were implanted and allowed to grow to an average volume of 100 mm³ before mice were randomized into treatment groups. Mice were dosed twice a week with Compound (100 μg) or PBS vehicle. Tumors were collected 24 hours after the second dose and dissociated for further analysis. The frequency of tetramer-positive CD8+ T cells producing TNF (FIG. 10A) or granzyme B after restimulation with PMA/lonomycin (FIG. 10B). P values are derived from t tests (*, P<0.05).

FIG. 11 is a graph showing that PD-1 monotherapy does not have anti-tumor activity in B16-F10. B16-F10 tumors were implanted and allowed to grow to an average volume of 100 mm³ before mice were randomized into treatment groups. Mice were dosed IP twice a week for two weeks with either vehicle (hollow circles; or anti-PD-1 (solid circles, 200 μg)). Tumor volume was measured over time. Data are representative of n=10 mice per group.

FIGS. 12A-12B shows the results of a baseline tumor-infiltrating lymphocyte analysis. MC38 and B16-F10 tumors were implanted and allowed to grow to an average volume of 100 mm³ before tumors were harvested for TIL analysis. FIG. 12A shows the gating strategy for the identification of various immune cell populations. FIG. 12B shows the frequency of various immune populations within the CD45+ population.

FIGS. 13A-13G shows the results of Compound 1 treatment in mice. MC38 tumor cells were implanted and allowed to grow to an average volume of 100-150 mm³ before mice were randomized into treatment groups. Labels in the legend represent the dose per mouse per dosing day. Mice were dosed IP twice a week for a total of four doses. FIG. 13A is a graph showing body weight measured over time. Average body weight of n=12 mice per group is shown. FIG. 13B is a graph showing tumor volume measured over time and is depicted as the mean+/−SEM. FIG. 13C is a graph showing the results of MC38 tumor bearing mice randomized and dosed with either vehicle, Compound 1, or a IL-2 prodrug missing the half life extension element. Tumor volume was measured over time and is depicted as the mean+/−SEM. FIG. 13D shows the results of mice dosed with equimolar amounts of recombinant human IL-2 (5 total doses over three days), WW0177 (2 doses over three days), or Compound 1 (2 doses over three days), before mice were injected intravenously with Evan's Blue solution. Evan's Blue extravasation into the lungs was measured 30 minutes following intravenous administration of the dye. FIG. 13E is a graph showing detection of either recombinant human IL-2 or recombinant mouse IL-2 by a human specific IL-2 ELISA. FIG. 13F is a graph showing detection of either Compound 1 or free IL-2 by a human specific IL-2 Alphalisa. FIG. 13G is a graph showing the Therapeutic Window representation of rhIL-2, WW0177, or Compound 1 in MC38 tumor-bearing mice. P values are derived from t tests (*, P<0.05; ***, P<0.001). ELISA, enzyme-linked immunosorbent assay; IL-2, interleukin-2; IP, intraperitoneal; MC, murine colon; rhIL-2, recombinant human IL-2; SEM, standard error of the mean; TW, therapeutic window.

FIGS. 14A-14C is a graph showing that Compound 1 is superior to equimolar amounts of recombinant human IL-2 at activating B16-F10 TILs. B16-F1 tumors were implanted and allowed to grow to an average volume of 100 mm³ before mice were randomized into treatment groups. Mice were dosed IP twice a week for two weeks with either vehicle (hollow circles; or anti-PD-1 (solid circles, 200 μg)). FIG. 14A is a graph showing tumor volume measured over time. Data are representative of n=10 mice per group, and are depicted as the mean+/−SEM. FIGS. 14B-14C show the results of tumors from mice treated with either the vehicle, Compound 1 (200 μg/dose) or equimolar amounts of recombinant human IL-2 that were harvested on Day 5. FIG. 14B shows quantitative analysis of CD25 expression and FIG. 14C Ki67 expression by various immune cell subsets. P values are derived from one way ANOVA analysis with multiple comparisons (*, P<0.05; **, P<0.01; ***, P<0.001; ****, P<0.0001).

FIGS. 15A-15C are graphs showing that a variant of Compound 1 containing a non-alpha IL-2 mutein (Compound 5) has no anti-tumor activity when compared to the same dose of Compound 1. FIG. 15A are graphs showing tumor volume measured overtime in MC38 tumor beating mice treated either with vehicle, Compound 1 (containing a native IL-2 payload, 100 μg/dose), or a variant of Compound 1 containing a non-alpha IL-2 mutein as a payload (100 μg/dose) (Compound 5). FIG. 15B are graphs depicting the frequency of tumor infiltrating tetramer+CD8+ T cells producing Granzyme B, IFNγ, or TNF on day 5. FIG. 15C are graphs showing the frequency of tumor infiltrating NK cells producing Granzyme B or IFNγ.

### 4. DETAILED DESCRIPTION

This disclosure relates to compositions and methods for treating cancer using an inducible IL-2 prodrug. The method generally comprises administering to a subject in need thereof an effective amount of an inducible IL-2 prodrug. The inducible IL-2 prodrug can be Compound 1, Compound 2, Compound 3, or Compound 4. The inducible IL-2 prodrug can be any one of Compounds 5-29. The inducible IL-2 prodrugs can selectively activate IL-2 in the tumor microenvironment and decreases IL-2-related toxicity while improving anti-tumor effects in patients with cancer. The inventors demonstrate and exemplify herein that inducible IL-2 is preferentially activated in tumor tissue by tumor-associated proteases, releasing active IL-2 in the tumor microenvironment. In vitro assays confirmed that the activity of an inducible IL-2 prodrug (Compound 1) is dependent on proteolytic activation, and an inducible IL-2 prodrug treatment results in complete rejection of established tumors in a cleavage-dependent manner.

The inventors show that treatment with inducible IL-2 prodrug triggers the activation of T cells and natural killer cells, and markedly shifts the immune activation profile of the tumor microenvironment, resulting in significant inhibition of tumor growth in syngeneic tumor models. The inventors further showed that inducible IL-2 prodrug minimizes the toxicity of IL-2 treatment in the periphery while retaining the full pharmacology of TL-2 in the tumor microenvironment, supporting its further development as a novel cancer immunotherapy treatment.

### A. IL-2 Prodrugs

The inducible IL-2 prodrug for use in the methods and compositions of this disclosure overcome the toxicity and short half-life problems that have severely limited the clinical use of cytokines in oncology. The inducible IL-2 prodrug contains an IL-2 polypeptide that has receptor agonist activity of native IL-2, including binding to and activating signaling through IL-2Rα/β/γ and IL-2Rβ/γ, but in the context of the inducible pro-drug, the cytokine receptor agonist activity is attenuated, and the circulating half-life is extended. The prodrug includes protease cleavage sequences, which are cleaved by proteases that are associated with, and are typically enriched or selectively present in, the tumor microenvironment. Thus, the inducible IL-2 prodrugs are preferentially (or selectively) and efficiently cleaved in the tumor microenvironment to release active IL-2, and to limit IL-2 activity substantially to the tumor microenvironment. The IL-2 that is released upon cleavage has a short half-life, which is substantially similar to the half-life of naturally occurring IL-2, further restricting IL-2 activity to the tumor microenvironment. Even though the half-life of the inducible IL-2 prodrug is extended, toxicity is dramatically reduced or eliminated because the circulating prodrug has attenuated TL-2 activity, and active IL-2 is restricted to the tumor microenvironment.

The inducible IL-2 prodrug comprises two polypeptide chains. The first polypeptide chain can comprise from amino to carboxy terminus: the IL-2 polypeptide—a protease cleavable linker—an anti-human serum albumin (HSA) binding single antibody variable domain—a linker that is preferably protease cleavable—VH and CH1 of an antibody that binds IL-2. The first polypeptide chain can comprise from amino to carboxy terminus: the IL-2 polypeptide—a protease cleavable linker—VH and CH1 of an antibody that binds TL-2—a linker that is preferably protease cleavable—an anti-human serum albumin (HSA) binding single antibody variable domain. The second polypeptide chain comprises a VL and CL of an antibody that binds IL-2 and that together with the VH and CH1 of the first polypeptide chain form a Fab that binds the IL-2 polypeptide. Compounds 1, 2, 3 and 4 are specific examples of inducible IL-2 prodrugs for use according to this disclosure. Compounds 1, 2, 3, and 4 and additional details regarding their activity is disclosed in WO2021/097376. Compounds 5-29 are additional examples of inducible IL-2 prodrugs for use according to this disclosure.

TABLE 1
Inducible IL-2 prodrugs

| IL-2 Prodrug | First Polypeptide | Second Polypeptide |
|---|---|---|
| Compound 1 | SEQ ID NO: 1 | SEQ ID NO: 5 |
| Compound 2 | SEQ ID NO: 2 | SEQ ID NO: 5 |
| Compound 3 | SEQ ID NO: 3 | SEQ ID NO: 5 |
| Compound 4 | SEQ ID NO: 4 | SEQ ID NO: 5 |
| Compound 5 | SEQ ID NO: 1 | SEQ ID NO: 8 |
| Compound 6 | SEQ ID NO: 1 | SEQ ID NO: 9 |
| Compound 7 | SEQ ID NO: 1 | SEQ ID NO: 10 |
| Compound 8 | SEQ ID NO: 1 | SEQ ID NO: 11 |
| Compound 9 | SEQ ID NO: 1 | SEQ ID NO: 12 |
| Compound 10 | SEQ ID NO: 13 | SEQ ID NO: 5 |
| Compound 11 | SEQ ID NO: 14 | SEQ ID NO: 5 |
| Compound 12 | SEQ ID NO: 15 | SEQ ID NO: 5 |
| Compound 13 | SEQ ID NO: 16 | SEQ ID NO: 5 |
| Compound 14 | SEQ ID NO: 17 | SEQ ID NO: 5 |
| Compound 15 | SEQ ID NO: 18 | SEQ ID NO: 5 |
| Compound 16 | SEQ ID NO: 19 | SEQ ID NO: 5 |
| Compound 17 | SEQ ID NO: 20 | SEQ ID NO: 5 |
| Compound 18 | SEQ ID NO: 21 | SEQ ID NO: 5 |
| Compound 19 | SEQ ID NO: 22 | SEQ ID NO: 5 |
| Compound 20 | SEQ ID NO: 23 | SEQ ID NO: 5 |
| Compound 21 | SEQ ID NO: 24 | SEQ ID NO: 5 |
| Compound 22 | SEQ ID NO: 25 | SEQ ID NO: 5 |
| Compound 23 | SEQ ID NO: 26 | SEQ ID NO: 5 |
| Compound 24 | SEQ ID NO: 27 | SEQ ID NO: 5 |
| Compound 25 | SEQ ID NO: 28 | SEQ ID NO: 5 |
| Compound 26 | SEQ ID NO: 29 | SEQ ID NO: 5 |
| Compound 27 | SEQ ID NO: 30 | SEQ ID NO: 5 |
| Compound 28 | SEQ ID NO: 31 | SEQ ID NO: 5 |
| Compound 29 | SEQ ID NO: 32 | SEQ ID NO: 5 |

Amino acid sequence variants of compounds 1, 2, 3 and 4, that retain attenuated IL-2 activity in the periphery and that release active IL-2 upon protease cleavage in the tumor microenvironment can also be used in accordance with this disclosure. For example, a prodrug can comprise a first polypeptide that has at least about 8000, at least about 8500, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:1 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:2 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:3 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:4 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO:5.

Amino acid sequence variants of compounds 5-29, that retain attenuated IL-2 activity in the periphery and that release active IL-2 upon protease cleavage in the tumor microenvironment can also be used in accordance with this disclosure.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 1 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 8.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 1 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 9.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 1 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 10.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 1 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 11.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 1 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 12.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 13 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 14 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 15 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 16 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 17 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 18 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 19 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 20 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 21 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 22 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 23 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 24 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 25 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 26 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 27 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 28 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 29 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 30 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 31 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

A prodrug can comprise a first polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 32 and a second polypeptide that has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% amino acid sequence identity with SEQ ID NO: 5.

For all amino acid sequence variant prodrugs it is preferred that the protease cleavage site contain no amino acid replacements, or only conservative amino acid replacements, so that the sequence variant prodrug is cleaved in the tumor microenvironment and releases IL-2 to substantially the same degree as the corresponding parental prodrug. Similarly, it is preferred that the complementarity determining regions of the anti-HAS single variable domain and the anti-IL2 Fab contain no amino acid replacements, or only conservative amino acid replacements, so that a) the serum half-life of the sequence variant prodrug is substantially the same as the corresponding parental prodrug, and b) the attenuation of IL-2 agonist activity of the sequence variant prodrug is substantially the same as the corresponding parental prodrug.

Exemplary amino acid substitutions are provided in Table 2.

TABLE 2
Exemplary amino acid substitutions

| | Exemplary |
|---|---|
| Amino Acid | Substitutions |
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

### B. Therapeutic Use and Pharmaceutical Compositions

This disclosure further relates to methods and compositions for treating cancer using an inducible IL-2 prodrug, optionally in combination with one or more additional therapeutic agents, such as a chemotherapeutic agents, cytokines, oncolytic viruses, immune-oncology agents, or a check point inhibitors (e.g., an anti-PD-1 antibody or an anti-PD-L1 antibody). Suitable chemotherapeutic agents (e.g., cyclophosphamide, mechlorethamine, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mitomycin, diaziquone (AZQ), cisplatin, carboplatin, oxaliplatin, procarbazine, hexamethylmelamine, methotrexate, pemetrexed, fluorouracil (e.g. 5-fluorouracil), capecitabine, cytarabine, gemcitabine, decitabine, azacitidine, fludarabine, nelarabine, cladribine, clofarabine, pentostatin, thioguanine, mercaptopurine, vincristine, vinblastine, vinorelbine, vindesine, vinflunine, paclitaxel, docetaxel, etoposide, teniposide, doxorubicin, daunorubicin, epirubicin, idarubicin, pirarubicin, aclarubicin, mitoxantrone, actinomycin, bleomycin, bisantrene, gemcitabine, cytarabine, and the like), Immune checkpoint proteins include, for example, PD-1 which binds ligands PD-L1 (B7-H1, CD274) and PD-L2 (B7-DC, CD273), CTLA-4 (CD152) which binds B7-1 (CD80) and B7-2 (CD86), LAG 3 (CD223) which binds Galectin3, LSECtin and FGL1; TIM3 (HAVCR2) which binds ligands CeacamI and Galectin9; TIGIT (VSTM3, WUCAM) which binds CD 112 and CD155; BTLA (CD272) which binds HVEM (TNFRSF14), B7-H3 (CD276), B7-H4 (VTCN1), VISTA (B7-H5), KIR, CD44 (2B4), CD160 (BY55) which bind HVEM; CD134 (TNRFSR4, OX40) which binds CD252 (OX-40L). Therapeutic agents, such as antibodies, that bind immune checkpoint proteins and inhibit their immunosuppressive activity include the anti-PD1 antibodies pembrolizumab (KEYTRUDA), dostarlimab (JEMPERLI), cemiplimab-rwlc (LIBATYO), nivolumab (OPDIVO), camrelizumab, tislelizumab, toripalimab, and sintilimab (TYVYT); the anti-PD-L1 antibodies avelumab (BAVENCIO), durvalumab (IMFINZI), and atezolizumab (TECENTRIQ); the anti-CTLA-4 antibody ipilimumab (YERVOY).

The inducible IL-2 prodrug and any additional therapeutic agents is typically administered systemically, for example by intravenous injection or preferably intravenous infusion. Other types of administration can be used, such as orally, parenterally, intravenous, intravenously, intra-articularly, intraperitoneally, intramuscularly, subcutaneously, intracavity, transdermally, intrahepatically, intracranially, nebulization/inhalation, by installation via bronchoscopy, or intratumorally.

The methods and compositions disclosed herein can be used to treat any suitable cancer, in particular solid tumors, such as sarcomas and carcinomas. For examples, the methods and compositions disclosed herein can be used to treat acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, glioblastoma, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. The non-small cell lung cancer (NSCLC) can be, for example, adenocarcinoma NSCLC, squamous cell NSCLC or large cell carcinoma NSCLC.

In certain embodiments, the methods and compositions disclosed herein can be used to treat adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, non-Hodgkin lymphoma, squamous carcinoma of the head and neck, malignant pleural mesothelioma, and Wilms tumor.

In certain preferred embodiments, the methods and compositions disclosed herein are used to treat melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), urothelial carcinoma, colorectal cancer or oesophageal carcinoma. In certain preferred embodiments, the methods and compositions disclosed herein are used to treat glioblastoma.

In certain preferred embodiments, the methods and compositions disclosed herein are used to treat Merkel Cell Carcinoma (MCC), Urothelial Carcinoma (UC), Renal Cell Carcinoma (RCC), non-small cell lung cancer (NSCLC), small cell lung cancer (SCLC), triple negative breast cancer (TNBC), endometrial cancer, cutaneous squamous cell carcinoma (CSCC), basal cell carcinoma (BCC), melanoma, malignant pleural mesothelioma, classical Hodgkin lymphoma (cHL), squamous cell carcinoma of the head and neck (SCCHN), hepatocellular carcinoma (HCC), esophageal squamous cell carcinoma (ESCC), non-squamous non-small cell lung cancer, or nasopharyngeal carcinoma (NPC).

Preferably, the methods and compositions disclosed herein are used to treat colon cancer, lung cancer, melanoma, renal cell carcinoma, or breast cancer.

In certain preferred embodiments, the methods and compositions disclosed herein are used to treat melanoma. As an example, the methods and compositions disclosed herein can be used to treat melanoma in subjects with unresectable or metastatic melanoma. As another example, the methods and compositions disclosed herein can be used for the adjuvant treatment of subjects with melanoma with involvement of lymph node(s) following complete resection.

In certain preferred embodiments, the methods and compositions disclosed herein are used to treat non-small cell lung cancer (NSCLC). As an example, the methods and compositions disclosed herein can be used to treat NSCLC in subjects with NSCLC expressing PD-L1 (e.g., Tumor Proportion Score (TPS)≥1%) as determined by an FDA-approved test, with no EGFR or ALK genomic tumor aberrations, and is: stage III where subjects are not candidates for surgical resection or definitive chemoradiation, or metastatic. As another example, the methods and compositions disclosed herein can be used to treat NSCLC in patients with metastatic NSCLC whose tumors express PD-L1 (TPS≥1%) as determined by an FDA-approved test, with disease progression on or after platinum-containing chemotherapy. As another example, the methods and compositions disclosed herein can be used in combination with pemetrexed and platinum chemotherapy, as first-line treatment of patients with metastatic nonsquamous NSCLC, with no EGFR or ALK genomic tumor aberrations. A s another example, the methods and compositions disclosed herein can be used in combination with carboplatin and either paclitaxel or paclitaxel protein-bound, as first-line treatment of patients with metastatic squamous NSCLC.

In certain preferred embodiments, the methods and compositions disclosed herein are used to treat SCLC. As an example, the methods and compositions disclosed herein can be used to treat SCLC in subjects with metastatic SCLC with disease progression on or after platinum-based chemotherapy and at least one other prior line of therapy.

In certain preferred embodiments, the methods and compositions disclosed herein are used to treat HNSCC. As an example, the methods and compositions disclosed herein can be used to treat HNSCC in subjects with metastatic or with unresectable, recurrent HNSCC whose tumors express PD-L1 (e.g., Combined Positive Score (CPS)≥1) as determined by an FDA-approved test. As another example, the methods and compositions disclosed herein can be used to treat HNSCC in subjects with recurrent or metastatic HNSCC with disease progression on or after platinum-containing chemotherapy. A s another example, the methods and compositions disclosed herein can be used in combination with platinum and fluorouracil for the first-line treatment of patients with metastatic or with unresectable, recurrent HNSCC.

In certain preferred embodiments, the methods and compositions disclosed herein are used to treat cHL. As an example, the methods and compositions disclosed herein can be used to treat cHL in subjects with relapsed or refractory cHL. As another example, the methods and compositions disclosed herein can be used to treat cHL in pediatric subjects with refractory cHL, or cHL that has relapsed after 2 or more lines of therapy.

In certain preferred embodiments, the methods and compositions disclosed herein are used to treat PMBCL. As an example, the methods and compositions disclosed herein can be used to treat PMBCL in subjects with refractory PMBCL, or in subjects who have relapsed after 2 or more prior lines of therapy.

In certain preferred embodiments, the methods and compositions disclosed herein are used to treat urothelial carcinoma. As an example, the methods and compositions disclosed herein can be used to treat urothelial carcinoma in subjects with locally advanced or metastatic urothelial carcinoma who are not eligible for cisplatin-containing chemotherapy and whose tumors express PD-L1 (e.g., Combined Positive Score (CPS)≥10) as determined by an FDA-approved test, or in subjects who are not eligible for any platinum-containing chemotherapy regardless of PD-L1 status. As another example, the methods and compositions disclosed herein can be used to treat urothelial carcinoma in subjects with locally advanced or metastatic urothelial carcinoma who have disease progression during or following platinum-containing chemotherapy or within 12 months of neoadjuvant or adjuvant treatment with platinum-containing chemotherapy. As another example, the methods and compositions disclosed herein can be used to treat urothelial carcinoma in subjects with Bacillus Calmette-Guerin (BCG)-unresponsive, high-risk, non-muscle invasive bladder cancer (NMIBC) with carcinoma in situ (CIS) with or without papillary tumors who are ineligible for or have elected not to undergo cystectomy.

In certain preferred embodiments, the methods and compositions disclosed herein are used to treat Microsatellite Instability-High (MSI-H) or Mismatch Repair Deficient (dMMR) Cancer. As an example, the methods and compositions disclosed herein can be used to treat MSI-H or dMMR cancer in subjects with unresectable or metastatic MSI-H or dMMR cancer wherein the solid tumors have progressed following prior treatment and the subject has no satisfactory alternative treatment options, or wherein the colorectal cancer has progressed following treatment with a fluoropyrimidine, oxaliplatin, and irinotecan.

In certain preferred embodiments, the methods and compositions disclosed herein are used to treat Microsatellite Instability-High (MSI-H) or Mismatch Repair Deficient (dMMR) Colorectal Cancer. As an example, the methods and compositions disclosed herein can be used to treat MSI-H or dMMR colorectal cancer in subjects with unresectable or metastatic MSI-H or dMMR colorectal cancer.

In certain preferred embodiments, the methods and compositions disclosed herein are used to treat gastric cancer. As an example, the methods and compositions disclosed herein can be used to treat gastric cancer in subjects with recurrent locally advanced or metastatic gastric or gastroesophageal junction adenocarcinoma whose tumors express PD-L1 (e.g., Combined Positive Score (CPS)≥1) as determined by an FDA-approved test, with disease progression on or after 2 or more prior lines of therapy including fluoropyrimidine- and platinum-containing chemotherapy and if appropriate, HER2/neu-targeted therapy.

In certain preferred embodiments, the methods and compositions disclosed herein are used to treat esophageal cancer. As an example, the methods and compositions disclosed herein can be used to treat esophageal cancer in subjects with locally advanced or metastatic esophageal or gastroesophageal junction (GEJ) (e.g., tumors with epicenter 1 to 5 centimeters above the GEJ) carcinoma that is not amenable to surgical resection or definitive chemoradiation, in combination with platinum- and fluoropyrimidine-based chemotherapy. As another example, the methods and compositions disclosed herein can be used to treat esophageal cancer in subjects with locally advanced or metastatic esophageal or gastroesophageal junction (GEJ) (e.g., tumors with epicenter 1 to 5 centimeters above the GEJ) carcinoma that is not amenable to surgical resection or definitive chemoradiation, after one or more prior lines of systemic therapy for patients with tumors of squamous cell histology that express PD-L1 (CPS≥10) as determined by an FDA-approved test.

In certain preferred embodiments, the methods and compositions disclosed herein are used to treat cervical cancer. As an example, the methods and compositions disclosed herein can be used to treat cervical cancer in subjects with recurrent or metastatic cervical cancer with disease progression on or after chemotherapy whose tumors express PD-L1 (e.g., Combined Positive Score (CPS)≥1) as determined by an FDA-approved test.

In certain preferred embodiments, the methods and compositions disclosed herein are used to treat HCC. As an example, the methods and compositions disclosed herein can be used to treat HCC in subjects who have been previously treated with sorafenib.

In certain preferred embodiments, the methods and compositions disclosed herein are used to treat MCC. As an example, the methods and compositions disclosed herein can be used to treat MCC in subjects with recurrent locally advanced or metastatic MCC.

In certain preferred embodiments, the methods and compositions disclosed herein are used to treat RCC. As an example, the methods and compositions disclosed herein can be used in combination with axitinib, for the first-line treatment of patients with advanced RCC.

In certain preferred embodiments, the methods and compositions disclosed herein are used to treat endometrial carcinoma. As an example, the methods and compositions disclosed herein can be used in combination with lenvatinib, for the treatment of subjects with advanced endometrial carcinoma that is not MSI-H or dMMR, who have disease progression following prior systemic therapy and are not candidates for curative surgery or radiation.

In certain preferred embodiments, the methods and compositions disclosed herein are used to treat Tumor Mutational Burden-High (TMB-H) Cancer. As an example, the methods and compositions disclosed herein can be used to treat TMB-H cancer in subjects with unresectable or metastatic tumor mutational burden-high (e.g., ≥10 mutations/megabase (mut/Mb)) solid tumors, as determined by an FDA-approved test, that have progressed following prior treatment and who have no satisfactory alternative treatment options.

In certain preferred embodiments, the methods and compositions disclosed herein are used to treat Cutaneous Squamous Cell Carcinoma (cSCC). As an example, the methods and compositions disclosed herein can be used to treat cSCC in subjects with recurrent or metastatic cutaneous squamous cell carcinoma that is not curable by surgery or radiation.

In certain preferred embodiments, the methods and compositions disclosed herein are used to treat Triple-Negative Breast Cancer (TNBC). As an example, the methods and compositions disclosed herein can be used in combination with chemotherapy, for the treatment of subjects with locally recurrent unresectable or metastatic TNBC whose tumors express PD-L1 (e.g., Combined Positive Score (CPS)≥10) as determined by an FDA approved test.

In certain preferred embodiments, the methods and compositions disclosed herein can be used to treat Merkel cell carcinoma (MCC). As an example, a combination comprising Avelumab can be used to treat MCC in subjects with metastatic MCC.

In certain preferred embodiments, the methods and compositions disclosed herein can be used to treat Urothelial Carcinoma (UC). As an example, a combination comprising avelumab can be used to treat UC in subjects with locally advanced or metastatic UC who have disease progression during or following platinum-containing chemotherapy. As another example, a combination comprising avelumab can be used to treat UC in subjects with locally advanced or metastatic UC who have disease progression within 12 months of neoadjuvant or adjuvant treatment with platinum-containing chemotherapy.

In certain preferred embodiments, the methods and compositions disclosed herein can be used to treat Renal Cell Carcinoma (RCC). As an example, a combination comprising avelumab and axitinib can be used in a subject with advanced RCC.

In certain preferred embodiments, the methods and compositions disclosed herein can be used to treat urothelial carcinoma (UC). As an example, a combination comprising Durvalumab can be used to treat UC in subjects with locally advanced or metastatic urothelial carcinoma who have disease progression during or following platinum-containing chemotherapy. As another example, a combination comprising Durvalumab can be used to treat UC in subjects with locally advanced or metastatic urothelial carcinoma who have disease progression within 12 months of neoadjuvant or adjuvant treatment with platinum-containing chemotherapy.

In certain preferred embodiments, the methods and compositions disclosed herein can be used to treat non-small cell lung cancer (NSCLC). As an example, a combination comprising Durvalumab can be used to treat NSCLC in subjects with unresectable, Stage III non-small cell lung cancer (NSCLC) whose disease has not progressed following concurrent platinum-based chemotherapy and radiation therapy.

In certain preferred embodiments, the methods and compositions disclosed herein can be used to treat small cell lung cancer (SCLC). As an example, a combination comprising Durvalumab can be used in combination with etoposide and either carboplatin or cisplatin, as first-line treatment of adult subjects with extensive-stage small cell lung cancer (ES-SCLC).

In certain preferred embodiments, the methods and compositions disclosed herein can be used to treat urothelial carcinoma (UC). As an example, a combination comprising Atezolizumab can be used to treat UC in adult subjects with locally advanced or metastatic urothelial carcinoma who are not eligible for cisplatin-containing chemotherapy and whose tumors express PD-L1 (e.g., PD-L1 stained tumor-infiltrating immune cells [IC] covering≥5% of the tumor area), as determined by an FDA-approved test, or are not eligible for any platinum-containing chemotherapy regardless of PD-L1 status, or have disease progression during or following any platinum-containing chemotherapy, or within 12 months of neoadjuvant or adjuvant chemotherapy.

In certain preferred embodiments, the methods and compositions disclosed herein can be used to treat NSCLC. As an example, a combination comprising Atezolizumab can be used to treat NSCLC in adult subjects with metastatic NSCLC whose tumors have high PD-L1 expression (e.g., PD-L1 stained≥50% of tumor cells [TC≥50%] or PD-L1 stained tumor-infiltrating immune cells [IC] covering≥10% of the tumor area [IC≥10%]), as determined by an FDA-approved test, with no EGFR or ALK genomic tumor aberrations. As another example, a combination comprising Atezolizumab can be used in combination with bevacizumab, paclitaxel, and carboplatin, for the first-line treatment of adult subjects with metastatic non-squamous NSCLC with no EGFR or ALK genomic tumor aberrations. As another example, a combination comprising Atezolizumab can be used in combination with paclitaxel protein-bound and carboplatin for the first-line treatment of adult subjects with metastatic non-squamous NSCLC with no EGFR or ALK genomic tumor aberrations. A s another example, a combination comprising Atezolizumab can be used to treat NSCLC in adult subjects with metastatic NSCLC who have disease progression during or following platinum-containing chemotherapy.

In certain preferred embodiments, the methods and compositions disclosed herein can be used to treat triple negative breast cancer (TNBC). As an example, a combination comprising Atezolizumab can be used in combination with paclitaxel protein-bound for the treatment of adult subjects with unresectable locally advanced or metastatic TNBC whose tumors express PD-L1 (e.g., PD-L1 stained tumor-infiltrating immune cells [IC] of any intensity covering≥1% of the tumor area), as determined by an FDA approved test.

In certain preferred embodiments, the methods and compositions disclosed herein can be used to treat Small cell lung cancer (SCLC). As an example, a combination comprising Atezolizumab can be used in combination with carboplatin and etoposide, for the first-line treatment of adult subjects with extensive-stage small cell lung cancer (ES-SCLC).

In certain preferred embodiments, the methods and compositions disclosed herein can be used to treat endometrial cancer. As an example, a combination comprising Dostarlimab can be used to treat endometrial cancer in adult subjects with mismatch repair deficient (dMMR) recurrent or advanced endometrial cancer, as determined by an FDA-approved test, that has progressed on or following prior treatment with a platinum-containing regimen.

In certain preferred embodiments, the methods and compositions disclosed herein can be used to treat cutaneous squamous cell carcinoma (CSCC). As an example, a combination comprising Cemiplimab-rwlc can be used to treat CSCC in subjects with metastatic cutaneous squamous cell carcinoma (mCSCC) or locally advanced CSCC (laCSCC) who are not candidates for curative surgery or curative radiation.

In certain preferred embodiments, the methods and compositions disclosed herein can be used to treat basal cell carcinoma (BCC). As an example, a combination comprising Cemiplimab-rwlc can be used to treat BCC in subjects with locally advanced BCC (laBCC) previously treated with a hedgehog pathway inhibitor or for whom a hedgehog pathway inhibitor is not appropriate.

In certain preferred embodiments, the methods and compositions disclosed herein can be used to treat NSCLC. As an example, a combination comprising Cemiplimab-rwlc can be used to treat NSCLC in subjects whose tumors have high PD-L1 expression (e.g., Tumor Proportion Score (TPS)≥50%) as determined by an FDA-approved test, with no EGFR, ALK or ROS1 aberrations, and is locally advanced where subjects are not candidates for surgical resection or definitive chemoradiation, or metastatic.

In certain preferred embodiments, the methods and compositions disclosed herein can be used to treat melanoma. As an example, a combination comprising Nivolumab can be used to treat melanoma in subjects with unresectable or metastatic melanoma, as a single agent or in combination with ipilimumab. As another example, a combination comprising Nivolumab can be used to treat melanoma in subjects with melanoma with lymph node involvement or metastatic disease who have undergone complete resection, in the adjuvant setting.

In certain preferred embodiments, the methods and compositions disclosed herein can be used to treat NSCLC. As an example, a combination comprising Nivolumab can be used to treat NSCLC in adult subjects with metastatic non-small cell lung cancer expressing PD-L1 (≥1%) as determined by an FDA-approved test, with no EGFR or ALK genomic tumor aberrations, as first-line treatment in combination with ipilimumab. As another example, a combination comprising NSCLC can be used to treat melanoma in adult subjects with metastatic or recurrent non-small cell lung cancer with no EGFR or ALK genomic tumor aberrations as first-line treatment, in combination with ipilimumab and 2 cycles of platinum-doublet chemotherapy. As another example, a combination comprising NSCLC can be used to treat melanoma in subjects with metastatic non-small cell lung cancer and progression on or after platinum-based chemotherapy.

In certain preferred embodiments, the methods and compositions disclosed herein can be used to treat malignant pleural mesothelioma. As an example, a combination comprising Nivolumab can be used to treat malignant pleural mesothelioma in adult subjects with unresectable malignant pleural mesothelioma, as first-line treatment in combination with ipilimumab.

In certain preferred embodiments, the methods and compositions disclosed herein can be used to treat RCC. As an example, a combination comprising Nivolumab can be used to treat RCC in subjects with intermediate or poor risk advanced renal cell carcinoma, as a first-line treatment in combination with ipilimumab. As another example, a combination comprising Nivolumab can be used to treat RCC in subjects with advanced renal cell carcinoma, as a first-line treatment in combination with cabozantinib. A s another example, a combination comprising Nivolumab can be used to treat RCC in subjects with advanced renal cell carcinoma who have received prior anti-angiogenic therapy.

In certain preferred embodiments, the methods and compositions disclosed herein can be used to treat classical Hodgkin lymphoma (cHL). As an example, a combination comprising Nivolumab can be used to treat cHL in adult subjects with cHL that has relapsed or progressed after autologous hematopoietic stem cell transplantation (HSCT) and brentuximab vedotin, or 3 or more lines of systemic therapy that includes autologous HSCT.

In certain preferred embodiments, the methods and compositions disclosed herein can be used to treat squamous cell carcinoma of the head and neck (SCCHN). As an example, a combination comprising Nivolumab can be used to treat SCCHN in subjects with recurrent or metastatic squamous cell carcinoma of the head and neck with disease progression on or after a platinum-based therapy.

In certain preferred embodiments, the methods and compositions disclosed herein can be used to treat urothelial carcinoma (UC). As an example, a combination comprising Nivolumab can be used to treat UC in subjects with locally advanced or metastatic urothelial carcinoma who have disease progression during or following platinum-containing chemotherapy or have disease progression within 12 months of neoadjuvant or adjuvant treatment with platinum-containing chemotherapy.

In certain preferred embodiments, the methods and compositions disclosed herein can be used to treat colorectal cancer. As an example, a combination comprising Nivolumab can be used to treat colorectal cancer in subjects with microsatellite instability-high (MSI-H) or mismatch repair deficient (dMMR) metastatic colorectal cancer that has progressed following treatment with a fluoropyrimidine, oxaliplatin, and irinotecan, as a single agent or in combination with ipilimumab.

In certain preferred embodiments, the methods and compositions disclosed herein can be used to treat hepatocellular carcinoma (HCC). As an example, a combination comprising Nivolumab can be used to treat HCC in subjects with HCC who have been previously treated with sorafenib, as a single agent or in combination with ipilimumab.

In certain preferred embodiments, the methods and compositions disclosed herein can be used to treat esophageal squamous cell carcinoma (ESCC). As an example, a combination comprising Nivolumab can be used to treat ESCC in subjects with unresectable advanced, recurrent or metastatic esophageal squamous cell carcinoma after prior fluoropyrimidine- and platinum-based chemotherapy.

In certain preferred embodiments, a combination comprising Camrelizumab can be used to treat cHL.

In certain preferred embodiments, a combination comprising Tislelizumab can be used to treat non-squamous non-small cell lung cancer. In certain preferred embodiments, a combination comprising Tislelizumab can be used to treat hepatocellular carcinoma (HCC).

In certain preferred embodiments, a combination comprising Toripalimab can be used to treat urothelial carcinoma. In certain preferred embodiments, a combination comprising Toripalimab can be used to treat melanoma. In certain preferred embodiments, a combination comprising Toripalimab can be used to treat nasopharyngeal carcinoma (NPC).

In certain preferred embodiments, a combination comprising Sintilimab can be used to treat non-squamous non-small cell lung cancer. In certain preferred embodiments, a combination comprising Sintilimab can be used to treat cHL.

The cancer to be treated using the methods and compositions of this disclosure can be metastatic cancer. The methods and compositions disclosed herein can be used to treat metastatic renal clear cell carcinoma or metastatic cutaneous malignant melanoma.

If desired, additional therapeutic agents can be administered to the subject. Typically such additional therapeutic agents are anti-cancer agents such as chemotherapeutic agents immunocheck point inhibitors, other cytokines (such as IL-12, inducible IL-12 prodrugs, inducible IFN, inducible IFN prodrugs, IL-2 or IL-2 prodrugs), angiogenesis inhibitors, antibody-drug conjugates (e.g., trastuzumab emtansine (KADCYLA), trastuzumab deruxtecan (ENHERTU), enfortumab vedotin (PADCEV), sacituzumab govitecan (TRODELVY), cellular therapies (e.g., CAR-T, TCT-T, T-cell therapy, such as tumor infiltrating lymphocyte (TIL) therapy), oncolytic viruses, radiation therapy and/or small molecules, as describride further herein.

The pharmaceutical compositions can take a variety of forms, e.g., liquid, lyophilized, and typically contain a suitable pharmaceutically acceptable carrier. Pharmaceutically acceptable carriers (or excipients) are the non-active ingredient components of the pharmaceutical composition and are not biologically or otherwise undesirable, i.e., the material is administered to a subject without causing undesirable biological effects or interacting in a deleterious manner with the other components of the pharmaceutical formulation or composition in which it is contained. Carriers are frequently selected to minimize degradation of the active ingredient and to minimize adverse side effects in the subject.

Suitable carriers and their formulations are described in Remington: The Science and Practice of Pharmacy, 21st Edition, David B. Troy, ed., Lippicott Williams & Wilkins (2005). Examples of the pharmaceutically-acceptable carriers include, but are not limited to, sterile water, saline, buffered solutions like Ringer's solution, and dextrose solution. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the chimeric polypeptides or nucleic acid sequences encoding the chimeric polypeptides to humans or other subjects.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. Typically, an appropriate amount of a pharmaceutically-acceptable salt is used in the formulation to render the formulation isotonic, although the formulation can be hypertonic or hypotonic if desired. The pH of the solution is generally about 5 to about 8 or from about 7 to 7.5.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

This disclosure also relates to a kit that includes a pharmaceutical composition that contains an a) inducible IL-2 prodrug composition, for example as a liquid composition or a lyophilized composition, in a suitable container (e.g., a vial, bag or the like), and b) a pembrolizumab composition, for example as a liquid composition or a lyophilized composition, in a suitable container (e.g., a vial, bag or the like). The kit can further include other components, such as sterile water or saline for reconstitution of lyophilized compositions.

### C. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

“Cytokine” is a well-known term of art that refers to any of a class of immunoregulatory proteins (such as interleukin or interferon) that are secreted by cells especially of the immune system and that are modulators of the immune system. Cytokine polypeptides that can be used in the fusion proteins disclosed herein include, but are not limited to transforming growth factors, such as TGF-α and TGF-β (e.g., TGFbeta1, TGFbeta2, TGFbeta3); interferons, such as interferon-α, interferon-β, interferon-γ, interferon-kappa and interferon-omega; interleukins, such as IL-1, IL-la, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21 and IL-25; tumor necrosis factors, such as tumor necrosis factor alpha and lymphotoxin; chemokines (e.g., C-X-C motif chemokine 10 (CXCL10), CCL19, CCL20, CCL21), and granulocyte macrophage-colony stimulating factor (GM-CS), as well as fragments of such polypeptides that active the cognate receptors for the cytokine (i.e., functional fragments of the foregoing). “Chemokine” is a term of art that refers to any of a family of small cytokines with the ability to induce directed chemotaxis in nearby responsive cells.

As used herein, the terms “inducible” refer to the ability of a protein, i.e. IL-2, IL-12, or IFN, that is part of a prodrug, to bind its receptor and effectuate activity upon cleavage of the prodrug in the tumor microenvironment. The inducible cytokine prodrugs disclosed herein have attenuated or no cytokine agonist activity, but upon cleavage in the tumor microenvironment release active cytokine.

“Attenuated” activity, means that biological activity and typically cytokine (i.e., IL-2, IL-12 or IFN) agonist activity is decreased as compared to the activity of the natural cytokine (i.e., IL-2, IL-12 or IFN). The inducible cytokine prodrugs disclosed herein have attenuated cytokine receptor agonists activity, that is at least about 10×, at least about 50×, at least about 100×, at least about 250×, at least about 500×, at least about 1000× or less agonist activity as compared to natural cytokine (i.e., IL-2, IL-12 or IFN). Upon cleavage in the tumor microenvironment, cytokine is released that is active. Typically, the cytokine that is released has cytokine receptor agonist activity that is at least about 10×, at least about 50×, at least about 100×, at least about 250×, at least about 500×, or at least about 1000× greater than the IL-2 receptor activating activity of the prodrug.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, “subject” can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, “patient” or “subject” may be used interchangeably and can refer to a subject with a disease or disorder (e.g. cancer). The term patient or subject includes human and veterinary subjects.

As used herein the terms “treatment”, “treat”, or “treating” refers to a method of reducing the effects of a disease or condition or symptom of the disease or condition. Thus, in the disclosed methods, treatment can refer to at least about 10%, at least about 20%, at least about 30%, at least about 40%, at least about 50%, at least about 60%, at least about 70%, at least about 80%, at least about 90%, or substantially complete reduction in the severity of an established disease or condition or symptom of the disease or condition, such as reduction in tumor volume, reduction in tumor burden, reduction in death. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms “prevent”, “preventing”, and “prevention” of a disease or disorder refers to an action, for example, administration of the chimeric polypeptide or nucleic acid sequence encoding the chimeric polypeptide, that occurs before or at about the same time a subject begins to show one or more symptoms of the disease or disorder, which inhibits or delays onset or exacerbation of one or more symptoms of the disease or disorder.

As used herein, references to “decreasing”, “reducing”, or “inhibiting” include a change of at least about 10%, of at least about 20%, of at least about 30%, of at least about 40%, of at least about 50%, of at least about 60%, of at least about 70%, of at least about 80%, of at least about 90% or greater as compared to a suitable control level. Such terms can include but do not necessarily include complete elimination of a function or property, such as agonist activity.

The term “sequence variant” refers to an amino acid sequence of a polypeptide that has substantially similar biological activity as a reference polypeptide but differs in amino acid sequence or to the nucleotide sequence of a nucleic acid that has substantially similar biological activity (e.g., encodes a protein with substantially similar activity) as a reference sequence but differs in nucleotide sequence. Typically the amino acid or nucleotide sequence of a “sequence variant” is highly similar (e.g. at least about 80% similar) to that of a reference sequence. Those of skill in the art readily understand how to determine the identity of two polypeptides or two nucleic acids. For example, the identity can be calculated after aligning the two sequences so that the identity is at its highest level over a defined number of nucleotides or amino acids. Optimal alignment of sequences for comparison may be conducted by the local identity algorithm of Smith and Waterman Adv. Appl. Math. 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The term “conservative amino acid substitution” is a term of art that refers to the replacement of an amino acid in a polypeptide with another amino acid that has similar biochemical properties, such as size, charge and hydrophobicity as a reference amino acid. It is well-known that conservative amino acid replacements in the amino acid sequence of a polypeptide frequently do not significantly alter the overall structure or function of the polypeptide. Conservative substitutions of amino acids are known to those skilled in the art. Conservative substitutions of amino acids can include, but not limited to, substitutions made amongst amino acids within the following groups: (a) M, I, L, V; (b) F, Y, W; (c) K, R, H; (d) A, G; (e) S, T; (f) Q, N; and (g) E, D. For instance, a person of ordinary skill in the art reasonably expect that an isolated replacement of a leucine with an isoleucine or valine, an aspartate with a glutamate, a threonine with a serine, or a similar replacement of an amino acid with a structurally related amino acid will not have a major effect on the biological activity of the resulting molecule.

The term “effective amount,” as used herein, refers to the amount of agent (e.g., inducible IL-2 prodrug) that is administered to achieve the desired effect under the conditions of administration, such an amount that reduces tumor size, reduces tumor burden, extends progression free survival or extends overall survival. The actual effective amount selected will depend on the particular cancer being treated and its stage and other factors, such as the subject's age, gender, weight, ethnicity, prior treatments and response to those treatments and other factors. Suitable amounts of inducible cytokine prodrug and any additional agents to be administered, and dosage schedules for a particular patient can be determined by a clinician of ordinary skill based on these and other considerations.

Preferably, the methods and compositions disclosed herein are used to treat colon cancer, lung cancer, melanoma, renal cell carcinoma, breast cancer, squamous carcinoma of the head and neck.

In certain preferred embodiments, the methods and compositions disclosed herein are used to treat melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), urothelial carcinoma, colorectal cancer or oesophageal carcinoma.

### 5. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

6. SEQUENCES

| SEQ ID | | |
|---|---|---|
| NO. | Name | Sequence |
| 1 | ACP457 | APTSSSTKKTQLQLEHLLLDLQMILNGINNYKNPKLTRMLTFKFYMPK |
| | (WW0621) | KATELKHLQCLEEELKPLEEVLNLAQSKNFHLRPRDLISNINVIVLELK |
| | | GSETTFMCEYADETATIVEFLNRWITFCQSIISTLTSGGPALFKSSFPPGS |
| | | EVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYY |
| | | CTIGGSLSVSSQGTLVTVSSGGGGSGGGGSGGGGSGGGGSGGGGSGG |
| | | GGSSGGPALFKSSFPPGSEVQLVESGGGLVQPGGSLRLSCAASGFTFSS |
| | | YTLAWVRQAPGKGLEWVAAIDSSSYTYSPDTVRGRFTISRDNAKNSLY |
| | | LQMNSLRAEDTAVYYCARDSNWDALDYWGQGTTVTVSSASTKGPSV |
| | | FPLAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKSC** |
| 2 | ACP378 | APTSSSTKKTQLQLEHLLLDLQMILNGINNYKNPKLTRMLTFKFYMPK |
| | (WW0520) | KATELKHLQCLEEELKPLEEVLNLAQSKNFHLRPRDLISNINVIVLELK |
| | | GSETTFMCEYADETATIVEFLNRWITFCQSIISTLTSGGPGPAGLYAQPG |
| | | SEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVY |
| | | YCTIGGSLSVSSQGTLVTVSSGGGGSGGGGSGGGGSGGGGSGGGGSG |
| | | GGGSSGGPGPAGLYAQPGSEVQLVESGGGLVQPGGSLRLSCAASGFTF |
| | | SSYTLAWVRQAPGKGLEWVAAIDSSSYTYSPDTVRGRFTISRDNAKNS |
| | | LYLQMNSLRAEDTAVYYCARDSNWDALDYWGQGTTVTVSSASTKGP |
| | | SVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSC |
| | | ** |
| 3 | WW0735 | APTSSSTKKTQLQLEHLLLDLQMILNGINNYKNPKLTRMLTFKFYMPK |
| | | KATELKHLQCLEEELKPLEEVLNLAQSKNFHLRPRDLISNINVIVLELK |
| | | GSETTFMCEYADETATIVEFLNRWITFCQSIISTLTSGGPGPAGLYAQPG |
| | | SEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVY |
| | | YCTIGGSLSVSSQGTLVTVSSGGGGSGGGGSGGGGSGGGGSGGGGSG |
| | | GGGSGGGGSGGGGSGGGGSEVQLVESGGGLVQPGGSLRLSCAASGFT |
| | | FSSYTLAWVRQAPGKGLEWVAAIDSSSYTYSPDTVRGRFTISRDNAKN |
| | | SLYLQMNSLRAEDTAVYYCARDSNWDALDYWGQGTTVTVSSASTKG |
| | | PSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSC |
| 4 | WW0736 | APTSSSTKKTQLQLEHLLLDLQMILNGINNYKNPKLTRMLTFKFYMPK |
| | | KATELKHLQCLEEELKPLEEVLNLAQSKNFHLRPRDLISNINVIVLELK |
| | | GSETTFMCEYADETATIVEFLNRWITFCQSIISTLTSGGPALFKSSFPPGS |
| | | EVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYY |
| | | CTIGGSLSVSSQGTLVTVSSGGGGSGGGGSGGGGSGGGGSGGGGSGG |
| | | GGSGGGGSGGGGSGGGGSEVQLVESGGGLVQPGGSLRLSCAASGFTF |
| | | SSYTLAWVRQAPGKGLEWVAAIDSSSYTYSPDTVRGRFTISRDNAKNS |
| | | LYLQMNSLRAEDTAVYYCARDSNWDALDYWGQGTTVTVSSASTKGP |
| | | SVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSC |
| 5 | ACP381 | DIQMTQSPSSLSASVGDRVTITCKAREKLWSAVAWYQQKPGKAPKSLI |
| | (WW0523) | YSASFRYSGVPSRFSGSGSGTDFTLTISSLQPEDFATYYCQQYYTYPYTF |
| | | GGGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| 6 | WW00865 | APASSSTKKTQLQLEHLLLDLQMILNGINNYKNPKLTRMLTAKFAMPK |
| | | KATELKHLQCLEEELKPLEEVLNGAQSKNFHLRPRDLISNINVIVLELK |
| | | GSETTFMCEYADETATIVEFLNRWITFAQSIISTLTsggpALFKSSFPpgsEV |
| | | QLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTI |
| | | GGSLSVSSQGTLVTVSSggggsggggsggggsggggsggggsggggssggpALFKSSF |
| | | PpgsEVQLVESGGGLVQPGGSLRLSCAASGFTFSSYTLAWVRQAPGKGL |
| | | EWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpe |
| | | pvtvswnsgaltsgvhtfpavlqssglyslssvvtvpssslgtqtyicnvnhkpsntkvdkrvepksc |
| 13 | WW01055 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggssggpALFKSSF |
| | | PpgsggggsEVQLVESGGGLVQPGNSLRLSCAASGFTFSKFGMSWVRQAP |
| | | GKGLEWVSSISGSGRDTLYAESVKGRFTISRDNAKTTLYLQMNSLRPE |
| | | DTAVYYCTIGGSLSVSSQGTLVTVSSggggsggggsggggsggggsggggsggggss |
| | | ggpALFKSSFPpgsEVQLVESGGGLVQPGGSLRLSCAASGFTFSSYTLAW |
| | | VRQAPGKGLEWVAAIDSSSYTYSPDTVRGRFTISRDNAKNSLYLQMNS |
| | | LRAEDTAVYYCARDSNWDALDYWGQGTTVTVSSastkgpsvfplapsskstsgg |
| | | taalgclvkdyfpepvtvswnsgaltsgvhtfpavlqssglyslssvvtvpssslgtqtyicnvnhkpsntkvd |
| | | krvepksc** |
| 14 | WW01056 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpALFKSSFPpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggggggssggpALFKSSFPpgsggggggggsEVQL |
| | | VESGGGLVQPGGSLRLSCAASGFTFSSYTLAWVRQAPGKGLEWVAAI |
| | | DSSSYTYSPDTVRGRFTISRDNAKNSLYLQMNSLRAEDTAVYYCARDS |
| | | NWDALDYWGQGTTVTVSSastkgpsvfplapsskstsggtaalgclvkdyfpepvtvswns |
| | | galtsgvhtfpavlqssglyslssvvtvpssslgtqtyicnvnhkpsntkvdkrvepksc** |
| 15 | WW01057 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpALFKSSFPpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggssggpALFKSSFPpgsggggsEVQLVESGGGL |
| | | VQPGGSLRLSCAASGFTFSSYTLAWVRQAPGKGLEWVAAIDSSSYTYS |
| | | PDTVRGRFTISRDNAKNSLYLQMNSLRAEDTAVYYCARDSNWDALDY |
| | | WGQGTTVTVSSastkgpsvfplapsskstsggtaalgclvkdyfpepvtvswnsgaltsgvhtfpav |
| | | lqssglyslssvvtvpssslgtqtyicnvnhkpsntkvdkrvepksc** |
| 16 | WW01058 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggssggpALFKSSF |
| | | PpgsggggsEVQLVESGGGLVQPGNSLRLSCAASGFTFSKFGMSWVRQAP |
| | | GKGLEWVSSISGSGRDTLYAESVKGRFTISRDNAKTTLYLQMNSLRPE |
| | | DTAVYYCTIGGSLSVSSQGTLVTVSSggggsggggssggpALFKSSFPpgsgggg |
| | | sggggsEVQLVESGGGLVQPGGSLRLSCAASGFTFSSYTLAWVRQAPGK |
| | | GLEWVAAIDSSSYTYSPDTVRGRFTISRDNAKNSLYLQMNSLRAEDTA |
| | | VYYCARDSNWDALDYWGQGTTVTVSSastkgpsvfplapsskstsggtaalgclvkdy |
| | | fpepvtvswnsgaltsgvhtfpavlqssglyslssvvtvpssslgtqtyicnvnhkpsntkvdkrvepksc** |
| 17 | WW01059 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggssggpALFKSSF |
| | | PpgsggggsEVQLVESGGGLVQPGNSLRLSCAASGFTFSKFGMSWVRQAP |
| | | GKGLEWVSSISGSGRDTLYAESVKGRFTISRDNAKTTLYLQMNSLRPE |
| | | DTAVYYCTIGGSLSVSSQGTLVTVSSggggssggpALFKSSFPpgsggggsEVQ |
| | | LVESGGGLVQPGGSLRLSCAASGFTFSSYTLAWVRQAPGKGLEWVAA |
| | | IDSSSYTYSPDTVRGRFTISRDNAKNSLYLQMNSLRAEDTAVYYCARD |
| | | SNWDALDYWGQGTTVTVSSastkgpsvfplapsskstsggtaalgclvkdyfpepvtvswn |
| | | sgaltsgvhtfpavlqssglyslssvvtvpssslgtqtyicnvnhkpsntkvdkrvepksc** |
| 18 | WW01163 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltGGGGSsggpALFKS |
| | | SFPpgsGGGGSEVQLVESGGGLVQPGGSLRLSCAASGFTFSSYTLAWVR |
| | | QAPGKGLEWVAAIDSSSYTYSPDTVRGRFTISRDNAKNSLYLQMNSLR |
| | | AEDTAVYYCARDSNWDALDYWGQGTTVTVSSastkgpsvfplapsskstsggtaa |
| | | lgclvkdyfpepvtvswnsgaltsgvhtfpavlqssglyslssvvtvpssslgtqtyicnvnhkpsntkvdkr |
| | | vepkscGGGGSsggpALFKSSFPpgsGGGGSEVQLVESGGGLVQPGNSLRLS |
| | | CAASGFTFSKFGMSWVRQAPGKGLEWVSSISGSGRDTLYAESVKGRFT |
| | | ISRDNAKTTLYLQMNSLRPEDTAVYYCTIGGSLSVSSQGTLVTVSS** |
| 19 | WW01164 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltGGGGSsggpALFKS |
| | | SFPpgsGGGGSEVQLVESGGGLVQPGGSLRLSCAASGFTFSSYTLAWVR |
| | | QAPGKGLEWVAAIDSSSYTYSPDTVRGRFTISRDNAKNSLYLQMNSLR |
| | | AEDTAVYYCARDSNWDALDYWGQGTTVTVSSastkgpsvfplapsskstsggtaa |
| | | lgclvkdyfpepvtvswnsgaltsgvhtfpavlqssglyslssvvtvpssslgtqtyicnvnhkpsntkvdkr |
| | | vepkscGGGGSGGGGSGGGGSGGGGSGGGGSEVQLVESGGGLVQPGNS |
| | | LRLSCAASGFTFSKFGMSWVRQAPGKGLEWVSSISGSGRDTLYAESVK |
| | | GRFTISRDNAKTTLYLQMNSLRPEDTAVYYCTIGGSLSVSSQGTLVTVS |
| | | S** |
| 20 | WW01060 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpHLFKSFPFpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggsggggsggggsggggsggggsggggssggpHLFKSFP |
| | | FpgsEVQLVESGGGLVQPGGSLRLSCAASGFTFSSYTLAWVRQAPGKGL |
| | | EWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpe |
| | | pvtvswnsgaltsgvhtfpavlqssglyslssvvtvpssslgtqtyicnvnhkpsntkvdkrvepksc** |
| 21 | WW01061 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpHKFKSKFPpgs |
| | | EVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYY |
| | | CTIGGSLSVSSQGTLVTVSSggggsggggsggggsggggsggggsggggssggpHKFK |
| | | SKFPpgsEVQLVESGGGLVQPGGSLRLSCAASGFTFSSYTLAWVRQAPG |
| | | KGLEWVAAIDSSSYTYSPDTVRGRFTISRDNAKNSLYLQMNSLRAEDT |
| | | AVYYCARDSNWDALDYWGQGTTVTVSSastkgpsvfplapsskstsggtaalgclvk |
| | | dyfpepvtvswnsgaltsgvhtfpavlqssglyslssvvtvpssslgtqtyicnvnhkpsntkvdkrvepksc |
| | | ** |
| 22 | WW01062 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpKLLFHLFPpgs |
| | | EVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYY |
| | | CTIGGSLSVSSQGTLVTVSSggggsggggsggggsggggsggggsggggssggpKLLF |
| | | HLFPpgsEVQLVESGGGLVQPGGSLRLSCAASGFTFSSYTLAWVRQAPG |
| | | KGLEWVAAIDSSSYTYSPDTVRGRFTISRDNAKNSLYLQMNSLRAEDT |
| | | AVYYCARDSNWDALDYWGQGTTVTVSSastkgpsvfplapsskstsggtaalgclvk |
| | | dyfpepvtvswnsgaltsgvhtfpavlqssglyslssvvtvpssslgtqtyicnvnhkpsntkvdkrvepksc |
| | | ** |
| 23 | WW01063 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpKKLFHIFPpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggsggggggggsggggsggggsggggssggpKKLFHIF |
| | | PpgsEVQLVESGGGLVQPGGSLRLSCAASGFTFSSYTLAWVRQAPGKGL |
| | | EWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpe |
| | | pvtvswnsgaltsgvhtfpavlqssglyslssvvtvpssslgtqtyicnvnhkpsntkvdkrvepksc** |
| 24 | WW01064 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpALFKSFPPpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggsggggsggggsggggsggggsggggssggpALFKSFP |
| | | PpgsEVQLVESGGGLVQPGGSLRLSCAASGFTFSSYTLAWVRQAPGKGL |
| | | EWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpe |
| | | pvtvswnsgaltsgvhtfpavlqssglyslssvvtvpssslgtqtyicnvnhkpsntkvdkrvepksc** |
| 25 | WW01065 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpALFKSLPPpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggsggggsggggsggggsggggsggggssggpALFKSLP |
| | | PpgsEVQLVESGGGLVQPGGSLRLSCAASGFTFSSYTLAWVRQAPGKGL |
| | | EWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpe |
| | | pvtvswnsgaltsgvhtfpavlqssglyslssvvtvpssslgtqtyicnvnhkpsntkvdkrvepksc** |
| 26 | WW01066 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpALFKSFPFpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggsggggsggggsggggsggggsggggssggpALFKSFP |
| | | FpgsEVQLVESGGGLVQPGGSLRLSCAASGFTFSSYTLAWVRQAPGKGL |
| | | EWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpe |
| | | pvtvswnsgaltsgvhtfpavlqssglyslssvvtvpssslgtqtyicnvnhkpsntkvdkrvepksc** |
| 27 | WW01067 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpAKFRHLFPpgs |
| | | EVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYY |
| | | CTIGGSLSVSSQGTLVTVSSggggsggggsggggggggsgggssggpAKFR |
| | | HLFPpgsEVQLVESGGGLVQPGGSLRLSCAASGFTFSSYTLAWVRQAPG |
| | | KGLEWVAAIDSSSYTYSPDTVRGRFTISRDNAKNSLYLQMNSLRAEDT |
| | | AVYYCARDSNWDALDYWGQGTTVTVSSastkgpsvfplapsskstsggtaalgclvk |
| | | dyfpepvtvswnsgaltsgvhtfpavlqssglyslssvvtvpssslgtqtyicnvnhkpsntkvdkrvepksc |
| | | ** |
| 28 | WW01068 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpALLKSIFPpgsE |
| | | VQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCT |
| | | IGGSLSVSSQGTLVTVSSggggsggggsggggsggggsggggggggssggpALLKSIF |
| | | PpgsEVQLVESGGGLVQPGGSLRLSCAASGFTFSSYTLAWVRQAPGKGL |
| | | EWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpe |
| | | pvtvswnsgaltsgvhtfpavlqssglyslssvvtvpssslgtqtyicnvnhkpsntkvdkrvepksc** |
| 29 | WW01069 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpAKLRSKFPpgs |
| | | EVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYY |
| | | CTIGGSLSVSSQGTLVTVSSggggsggggsggggsggggggggsggggssggpAKLR |
| | | SKFPpgsEVQLVESGGGLVQPGGSLRLSCAASGFTFSSYTLAWVRQAPG |
| | | KGLEWVAAIDSSSYTYSPDTVRGRFTISRDNAKNSLYLQMNSLRAEDT |
| | | AVYYCARDSNWDALDYWGQGTTVTVSSastkgpsvfplapsskstsggtaalgclvk |
| | | dyfpepvtvswnsgaltsgvhtfpavlqssglyslssvvtvpssslgtqtyicnvnhkpsntkvdkrvepksc |
| | | ** |
| 30 | WW01070 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpPLYAKLKSSFP |
| | | pgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAV |
| | | YYCTIGGSLSVSSQGTLVTVSSggggsggggsggggsggggsggggsggggssggpPL |
| | | YAKLKSSFPpgsEVQLVESGGGLVQPGGSLRLSCAASGFTFSSYTLAWV |
| | | RQAPGKGLEWVAAIDSSSYTYSPDTVRGRFTISRDNAKNSLYLQMNSL |
| | | RAEDTAVYYCARDSNWDALDYWGQGTTVTVSSastkgpsvfplapsskstsggt |
| | | aalgclvkdyfpepvtvswnsgaltsgvhtfpavlqssglyslssvvtvpssslgtqtyicnvnhkpsntkvd |
| | | krvepksc** |
| 31 | WW01071 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpPLAQKVKSSF |
| | | PpgsEVQLVESGGGLVQPGNSLRLSCAASGFTFSKFGMSWVRQAPGKG |
| | | LEWVSSISGSGRDTLYAESVKGRFTISRDNAKTTLYLQMNSLRPEDTA |
| | | VYYCTIGGSLSVSSQGTLVTVSSggggsggggsggggggggggggsggggssggpP |
| | | LAQKVKSSFPpgsEVQLVESGGGLVQPGGSLRLSCAASGFTFSSYTLAW |
| | | VRQAPGKGLEWVAAIDSSSYTYSPDTVRGRFTISRDNAKNSLYLQMNS |
| | | LRAEDTAVYYCARDSNWDALDYWGQGTTVTVSSastkgpsvfplapsskstsgg |
| | | taalgclvkdyfpepvtvswnsgaltsgvhtfpavlqssglyslssvvtvpssslgtqtyicnvnhkpsntkvd |
| | | krvepksc** |
| 32 | WW01072 | aptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltsggpPLAQKLRSSFP |
| | | pgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAV |
| | | YYCTIGGSLSVSSQGTLVTVSSggggsggggsggggsggggsggggsggggssggpPL |
| | | AQKLRSSFPpgsEVQLVESGGGLVQPGGSLRLSCAASGFTFSSYTLAWV |
| | | RQAPGKGLEWVAAIDSSSYTYSPDTVRGRFTISRDNAKNSLYLQMNSL |
| | | RAEDTAVYYCARDSNWDALDYWGQGTTVTVSSastkgpsvfplapsskstsggt |
| | | aalgclvkdyfpepvtvswnsgaltsgvhtfpavlqssglyslssvvtvpssslgtqtyicnvnhkpsntkvd |
| | | krvepksc** |
| 7 | MulV p15E | KSPWFTTL |
| | peptide | |
| 8 | WW00524 | DIQMTQSPSSLSASVGDRVTITCKVTEKVWGNVAWYQQKPGKAPISLI |
| | | YSPSLRKSGVPSRFSGSGSGTDFTLTISSLQPEDFATYYCQQYYTYPYTF |
| | | GGGTKVEIKrtvaapsvfifppsdeqlksgtasvvcllnnfypreakvqwkvdnalqsgnsqesvteq |
| | | dskdstyslsstltlskadyekhkvyacevthqglsspvtksfnrgec ** |
| 9 | WW00590 | DIQMTQSPSSLSASVGDRVTITCKSSEKLWANVAWYQQKPGKAPKsLI |
| | | YSASFRYSGVPSRFSGSGSGTDFTLTISSLQPEDFATYYCQQYYTYPYTF |
| | | GGGTKVEIKrtvaapsvfifppsdeqlksgtasvvcllnnfypreakvqwkvdnalqsgnsqesvteq |
| | | dskdstyslsstltlskadyekhkvyacevthqglsspvtksfnrgec ** |
| 10 | WW00592 | DIQMTQSPSSLSASVGDRVTITCKAREKLWSAVAWYQQKPGKAPKLLI |
| | | YSASFRYSGVPSRFSGSGSGTDFTLTISSLQPEDFATYYCQQYYTYPYTF |
| | | GGGTKVEIKrtvaapsvfifppsdeqlksgtasvvcllnnfypreakvqwkvdnalqsgnsqesvteq |
| | | dskdstyslsstltlskadyekhkvyacevthqglsspvtksfnrgec ** |
| 11 | WW01073 | diqmtqspsslsasvgdrvtitckasEKLWSnvgwyqqkpgkapkSliysasfrysgvpsrfsgsgsgt |
| | | dftltisslqpedfatyycqqyytypytfgggtkveikrtvaapsvfifppsdeqlksgtasvvcllnnfyprea |
| | | kvqwkvdnalqsgnsqesvteqdskdstyslsstltlskadyekhkvyacevthqglsspvtksfnrgec** |
| 12 | WW01074 | diqmtqspsslsasvgdrvtitckasEKLWSnvgwyqqkpgkapkLliysasfrysgvpsrfsgsgsgt |
| | | dftltisslqpedfatyycqqyytypytfgggtkveikrtvaapsvfifppsdeqlksgtasvvcllnnfyprea |
| | | kvqwkvdnalqsgnsqesvteqdskdstyslsstltlskadyekhkvyacevthqglsspvtksfnrgec** |

### 7. EXAMPLES

The following are examples of methods and compositions of the invention. It is understood that various other embodiments may be practiced, given the general description provided herein.

### 1.1 Materials and Methods

### Cell Lines

MC3 8 and B 16-F10 cell lines were obtained from ATCC and were regularly checked for pathogen contamination. All cell lines were grown and maintained according to ATCC guidelines and kept in culture for no longer than two weeks. Frozen MC38 or B16-F10 cells were thawed and maintained for 1-3 passages in DMEM (ThermoFisher Scientific) supplemented with 1000 heat-inactivated FCS (Gibco) and 1× penicillin/streptomycin (Gibco). Prior to tumor implantation, cells were washed twice with PBS and counted. Cells were inoculated in PBS (efficacy studies) or 50% Matrigel (TIL, harvests, Corning).

### Mice, Tumor Implantations, and In Vivo Dosing

All mouse in vivo work was performed in accordance with current regulations and standards of the U.S. Department of Agriculture and the NIH at Charles River Laboratories (Morrisville, NC and Worcester, MA). Female, 6-8 week-old C57Bl/6 mice from Charles River Laboratories were shaved on their flank 1 day prior to tumor cell implantation. A total of 5×10⁵ MC38 or 1×10⁵ B16-F10 cells were injected subcutaneously and monitored for tumor growth. Extra mice were implanted in order to have sufficiently sized tumors for randomization. Tumor volume was monitored until the group average was 100-150 mm³, and mice were randomized into treatment groups on Day 0. Mice receiving inducible IL-2 prodrugs were dosed twice a week. Mice receiving rhIL-2 were dosed twice a day for 5 days before receiving a 2-day break (5/2 regimen). In studies where PD-1 blockade was used, mice were dosed with anti-PD-1 (200 μg, clone RMP1-14, BioXCell) on a twice-weekly schedule. In studies using FTY720, mice were initially dosed with 25 μg on the first dose, then treated daily with 10 μg per dose throughout the course of the experiment.

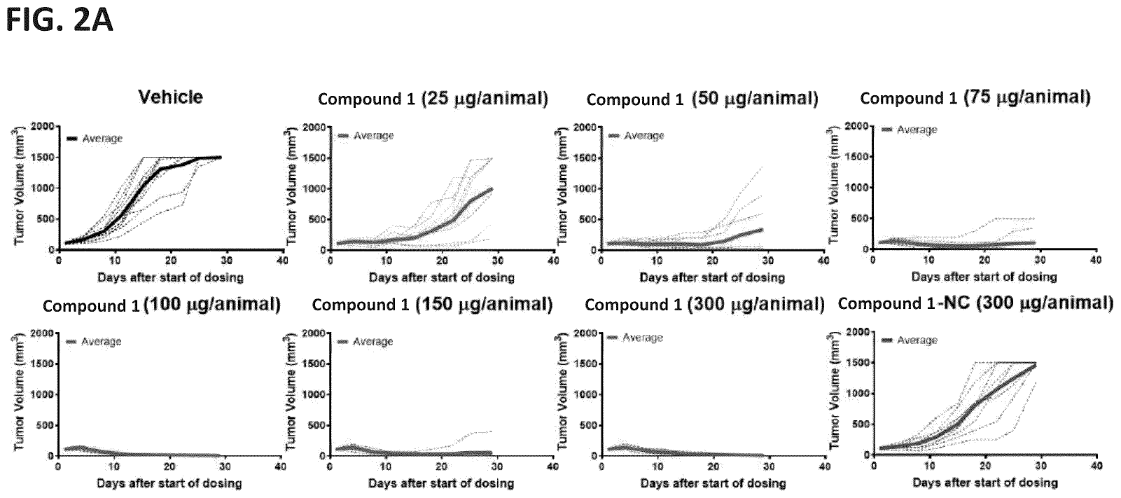

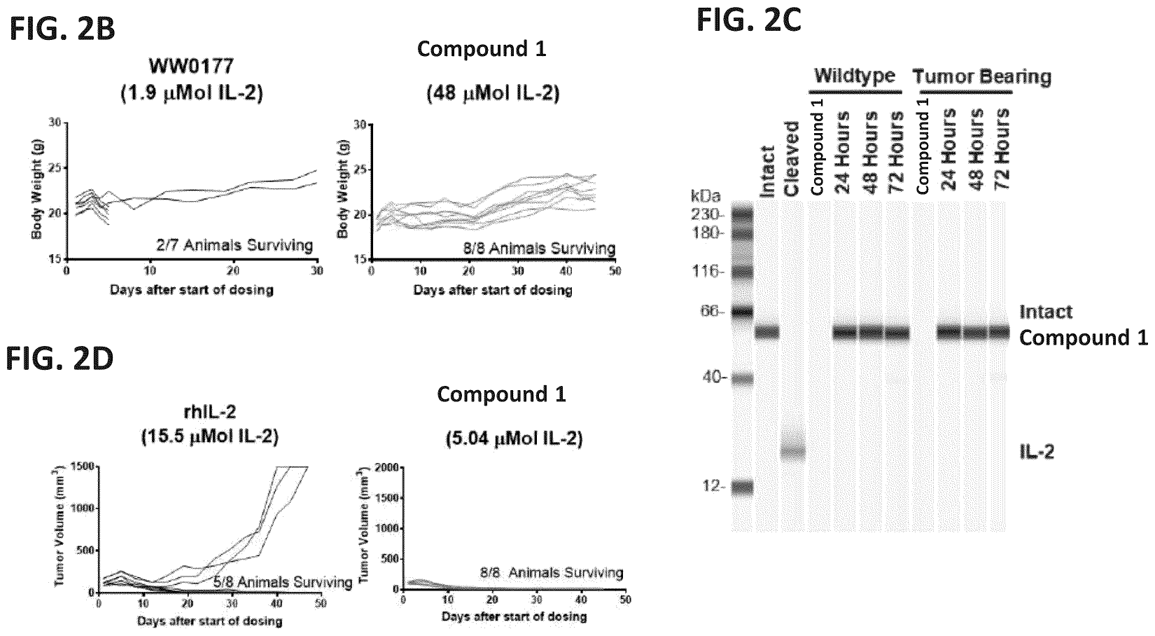

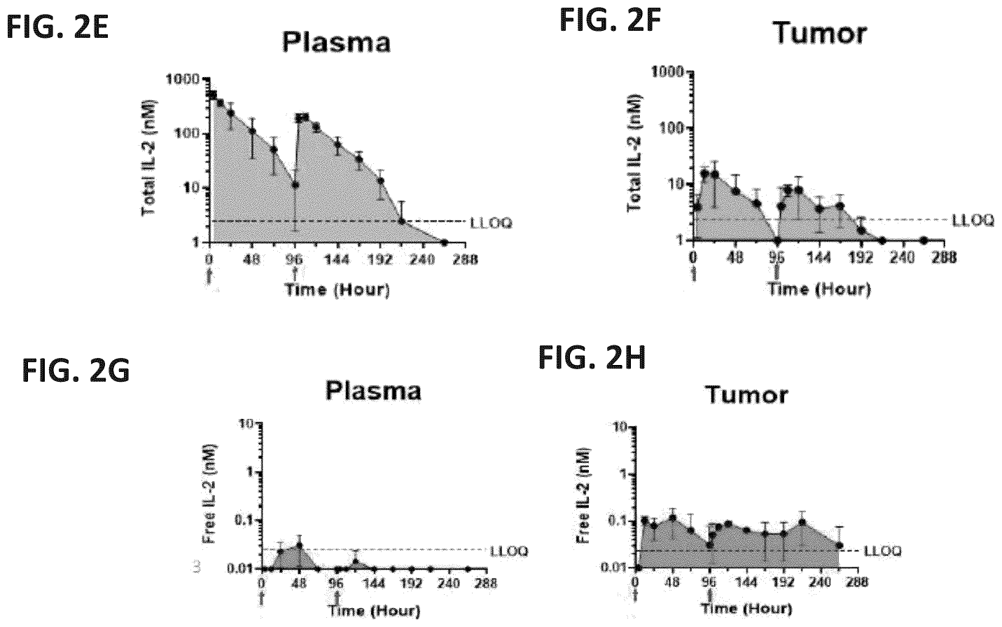

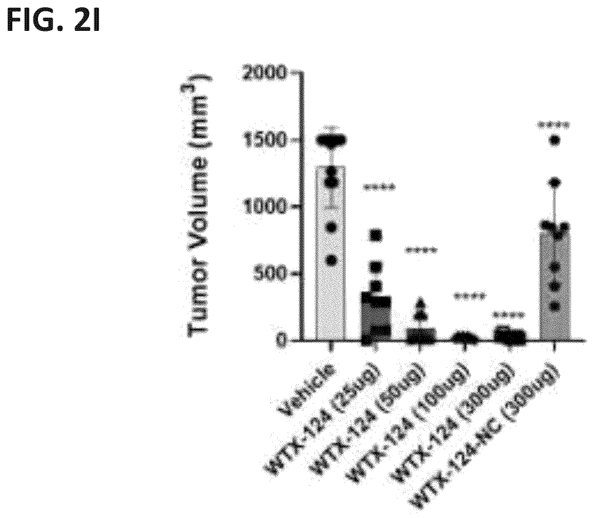

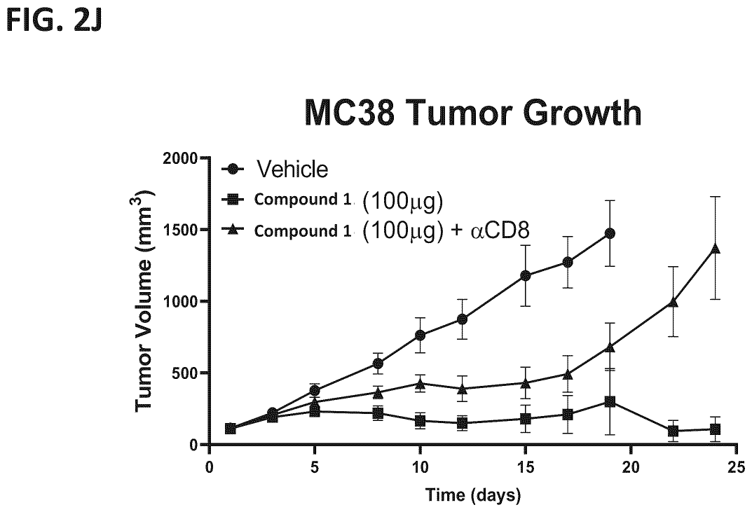

In some studies antitumor activity was assessed in mice in which CD8+ cells were depleted. In those studies mice were dosed with anti-CD8 antibody (200 μg/dose, clone 2.43 from Bio X Cell) twice a week via intraperitoneal injection. The average tumor volume for each group is displayed as the mean+/−the SEM (FIG. 2I). The results showed that repletion of CD8+ cells reduced the anti-tumor effect of Compound 1.

In some studies MC38 bearing mice were treated with either vehicle, Compound 1 (containing a native IL-2 payload at 100 μg/dose), or an inducible form of an IL-2 mutein that does not bind IL-2 receptor a upon cleavage but does bind IL-2 receptor beta-gamma receptors (Compound 5) at 100 μg/dose. Compound 5 comprises a first polypeptide having SEQ ID NO: 6 and a second polypeptide comprising SEQ ID NO: 5 (FIGS. 15A-15C). The results demonstrated, that the non-alpha mutein did not have significant anti-tumor activity in the model.

All treatments were administered by intraperitoneal injection, and mice were dosed for 2 weeks unless otherwise noted. Body weight and tumor volume were both measured twice weekly for the duration of the study. Tumors were measured in two dimensions using calipers, and volume was calculated using the formula: Tumor volume (mm3) [(w2×l) 2] where w=width and l=length, in mm, of a tumor. Mice were kept on study until tumors reached 1500 mm3, or the study reached the termination point at Day 45. In some instances, mice with complete regressions were saved for later memory experiments.

### Murine Vascular Leak Syndrome Model

Murine VLS experiments were performed in accordance with current regulations and standards of the U.S. Department of Agriculture and the NIH at Biomodels LLC (Waltham, MA). Female, 8-10 week-old C57Bl/6 were dosed with equimolar amounts of either recombinant human IL-2 (100 g/dose, given 7 times over four days), WW0177 (given twice on DO and D3), or Compound 1 (given twice on DO and D3) by intraperitoneal injection. On Day 3, animals were given intravenous injections of Evan's Blue dye, and animals were perfused thirty minutes later with 50 mL of saline with heparin at a rate of 10 mL/minute. Lungs were harvested and placed in formamide at 37° C. for 24 hours. After 24 hours, Evan's Blue extravasation into the lungs was measured using a spectrometer by measuring absorbance at 620 nm and 650 nm and comparing the absorbance values to a freshly prepared standard curve of Evan's Blue dye.

### Inducible IL-2 Prodrug Production, Protease Activation, and SDS Page Analysis

Compound 1, recombinant human IL-2, and Compound 1-NC were produced. Proteins were expressed using the Expi293 expression system from Life Technologies according to the manufacturer's protocol. On Day 4 post-transfection, the cultures were spun down, filtered with 0.2 m bottle top filters, and left to rotate overnight in the presence of MabSelect resin. The following day, the culture/resin mixture was applied to a gravity column and the resin was washed with PBS (TEKNOVA, endotoxin tested). Proteins were eluted with 200 mM acetic acid pH 3.5, 50 mM NaCl and neutralized with 1 M Tris pH 8. Elutions were pooled, dialyzed, concentrated, aliquoted and stored for future use at −80° C. WTX-124 was dialyzed into 20 mM histidine pH 6, 150 mM NaCl, while recombinant human IL-2 and WW0177 were dialyzed against 1×PBS. Extinction coefficients were determined for each protein theoretically using SnapGene (v 5.0.7) and protein concentration was determined by A280. For SDS page gels, 3 ag of protein was loaded on a 16% Tris-Glycine gel (ThermoFisher) under non-reducing conditions.

### HEK-Blue IL-2 Reporter Assay

The HEK-Blue IL-2 reporter cell assay was performed according to the manufacturer's protocol (Invivogen). On assay Day 1, the cells were rinsed, resuspended in media containing 1.5% human serum albumin and plated at a concentration of 5×10⁴ cells per well in a 96-well flat bottom plate. Titrated amounts of intact and protease-activated (cleaved) inducible IL-2 proteins or rhIL-2 were added to the cells to generate a full dose-response curve. On Day 2, SEAP levels measured according to the manufacturer's protocol.

### Human and Marine Primary Cell Assays

Human PBMCs were isolated using Ficoll-Paque Plus (GE Healthcare) according to the manufacturer's protocol and frozen in Recovery Cell Culture Freezing Media (Gibco) for later use. To generate activated T cells (Tblasts), PBMCs were thawed, counted, and stimulated with 5 g/mL of PHA (Sigma-Aldrich) for 72 hours before being frozen for later use. To measure intact or protease-activated (cleaved) inducible IL-2 protein activity, Tblasts were plated in a 96-well round bottom plate, and titrated amounts of intact or protease-activated (cleaved) inducible IL-2 proteins or rhIL-2 were added to the cells to generate a full dose-response curve. After 72 hours, proliferation was measured using Cell Titer glow reagent (Promega) according to the manufacturer's protocol.

For murine Tblast experiments, splenocytes were thawed, washed, and stimulated with 2 g/mL of Concanavalin A (Sigma-Aldrich) for 72 hours before being frozen in Recovery Cell Culture Freezing Media (Gibco). T cell activation was performed in complete media (RPMI-1640 media supplemented with 10% FBS, 100 units/mL of penicillin, 100 μg/mL streptomycin and 0.1% 2-mercaptoethanol). To measure inducible IL-2 protein activity, murine Tblasts were plated in a 96-well round bottom plate. Titrated amounts of intact or protease-activated (cleaved) inducible IL-2 protein or rhIL-2 were added to the cells to generate a full dose-response curve. After 72 hours, proliferation was measured using Cell Titer glow reagent (Promega) according to the manufacturer's protocol.

### Inducible IL-2 Stability in Murine Plasma and Human Serum

Whole blood from 6-8 week-old female C57Bl/6 mice was used to generate plasma. Human serum was purchased from BioIVT. On Day 1 of the assay, inducible IL-2 was added to either the murine plasma or human serum before the samples were mixed and divided into three aliquots, which were incubated at 37° C. for the indicated times before being frozen for later analysis. To assess the enzymatic processing of inducible IL-2, samples were thawed, and inducible IL-2 cleavage was assessed using western blot analysis against human IL-2. Intact and protease-activated inducible IL-2 were included as positive and negative controls.

Western blot analysis was performed using the JESS system (Protein Simple) according to the manufacturer's protocol. The primary anti-human IL-2 antibody was purchased from R&D Systems (AF-202-NA) and the anti-goat secondary antibody was purchased from Jackson Labs (AB 2338513). Samples and antibodies were loaded into a 12-230 kDA Jess separation module and run using a Jess system set to the standard settings for chemiluminescence. Analysis of the resulting western blot was performed using Compass for Simple Western Software (v4.1.0).

### Pharmacokinetic Analysis

Plasma and tumor samples were collected at indicated time points by Charles River Laboratories (Morrisville, North Carolina) and shipped on dry ice where they were stored at −80° C. MC38 tumor lysates were generated by homogenizing each tumor with a Qiagen TissueRuptor homogenizer with disposable probes (Qiagen) in ice cold Lysis Buffer (1× Tris Buffered Saline, 1 mM EDTA, 1% Triton X-100, with protease inhibitors in diH2O). Plasma and tumor lysates were analyzed using the BioLegend IL-2 ELISA (431804), which detects both intact inducible IL-2 as well as free IL-2, as per manufacturer's instructions. Intact inducible IL-2 was used to generate a 12-point standard curve. To specifically analyze the level of free IL-2, samples were measured using an IL-2 AlphaLISA (PerkinElmer, AL221C), which detects free human IL-2 but not intact inducible IL-2 due to competition with the inactivation domain. All AlphaLISAs were performed according to manufacturer's instructions and analyzed on a Perkin Elmer Enspire reader and software.

### Tumor Digestions and NanoString Analysis

MC38 and B16-F10 tumors were chopped into small pieces (<5 mm3) in phenol-free RPMI-1640 (Thermofisher) before being enzymatically digested with Collagenase IV (3 mg/mL, Gibco) at 37° C. for 35 minutes while shaking. After digestion, tumor samples were mechanically dissociated through a 70 μM cell strainer. For flow cytometry analysis involving effector cytokines, samples were restimulated for 4 hours at 37° C. in complete media containing phorbol 12-myristate 13-acetate (50 ng/mL, Sigma-Aldrich), Ionomycin (1 μg/mL, Sigma-Aldritch), and 1× Brefeldin A (eBioscience). For NanoString analysis, 5×10⁵ cells were frozen in 100 μL of RLT Lysis buffer (Qiagen). RNA samples were shipped to LakePharma, and analyzed using the nCounter Mouse PanCancer Immune Profiling Codeset Panel with the nCounter FLEX analysis system. NanoString analysis was performed using nSolver™ Software with the Advanced Analysis module installed.

### Flow Cytometry

All cell staining was performed in 96-well round bottom plates using FACs Buffer (PBS+0.5% BSA) or 1× Permeablization Buffer (eBioscience) where appropriate. Cells were first treated with FC block (BioLegend) at room temperature before tetramer staining was performed for 20 minutes at room temperature. After tetramer staining, cells were washed and then stained with a master mix of extracellular antibodies for 20 minutes at 4° C. Cells were then washed and fixed/permeabilized overnight using the eBioscience™ Foxp3 Transcription Factor Staining Buffer Set according to the manufacturer's protocol. The next day, samples were washed with Perm Buffer and stained with intracellular markers for 20 minutes at 4° C. Cells were then washed and analyzed on a Cytek Aurora system. Fluorescence minus one (FMO) and single stain controls were included for all stains. In some instances, OneComp ebeads™ (Thermofisher) were stained alongside cells to act as single stain controls. Individual cell populations were defined as described by the gating strategy in FIGS. 12A-12B. When flow cytometry was used to assess effector cytokine production, cells were restimulated with PMA (50 ng/mL, Sigma Aldrich) and Ionomycin (1 μg/mL, Sigma Aldrich) in the presence of 1× Breldin A (Thermofisher Scientific) for 4 hours in complete media at 37° C. prior to staining. Flow cytometry fluorescent dye-conjugated antibodies to the following proteins were purchased from Biolegend: CD8a APC, clone 53-67; CD4 BV650, clone RM4-5; CD3 AF700, clone 17A2; CD45 BV605, clone 30-F11; CD49b APC/Cy7, clone DX5; CD25 BV421, clone PC61; CD25 APC/Fire 750, clone PC61; Ki67 PeCy7, clone 16A8; Ki67 AF700, clone 16A8; granzyme B FITC, clone GB11; IFN7 PE, clone XMG1.2; F4/80 Pe/Dazzle 594, clone BM8; CD3 Complex PeCy7, clone 17A2; FC Block, clone 93. Flow cytometry fluorescent dye-conjugated antibodies to the following proteins were purchased from eBioscience: CD45 BUV395, clon30-F11; CD4 BUV496, clone GK1.5; CD8 BUV563, 53.6-7; TNF BV750, clone MP6-XT22; CD49B Pe-Cy5, clone DX5, FoxP3 AF488, clone FJK-16s; FoxP3 eFlour450, clone FJK-16s. The fluorescent dye-conjugated tetramer against the MulV p15E peptide KSPWFTTL (SEQ ID NO: 7) was purchased from ThermoFisher Scientific (50-168-9385). The Live/Dead Blue Dye was also purchased from ThermoFisher Scientific (L23105).

### Ex Vivo Inducible IL-2 Prodrug Processing Assay

Primary human healthy cells were purchased from either ATCC, Lonza, or Zen-Bio, and cultured according to the manufacturer's protocol. Dissociated human tumor samples were purchased from Discovery Life Sciences. These samples are generated from primary human tumor samples that were surgically removed and enzymatically digested on site prior to being frozen. All purchased samples were shipped on dry ice and were stored in a liquid nitrogen freezer.

To examine inducible IL-2 prodrug processing, samples were thawed, washed, and counted. Cells were then resuspended in X-Vivo 15 media containing either Compound 1, Compound 1-NC, or pre-cut Compound 1. Inducible IL-2 prodrug were incubated with cells for 48 hours before cell culture supernatants were collected and frozen for later analysis. The IL-2 Bioassay (Promega), which utilizes thaw-and-use IL-2 reporter cells, was used to assess the IL-2 activity in the cell culture supernatants (Catalog #JA2201/JA2205). This bioassay was used according to the manufacturer's protocol. Relative luminescence unit (RLU) values were translated into percent full activity using the following equation:

$$Percent of Full Activity = ( 1 - \frac{( Sample - Uncleavable Ctrl )}{( Cleaved Ctrl - Uncleavable Ctrl )} ) * 100 \qquad Eq . 1$$

### Data Representations and Statistics

For murine tumor experiments, mice were implanted with the respective tumor cell lines such that each group had at least n=8 mice per group at the time of randomization and the initiation of dosing. In order to have sufficient animals to appropriately randomize based on tumor size, the total number of mice implanted was calculated by adding 30% to the total number of animals needed on study. Sample size was determined by previous experience with this model, and tumor measurements were made in an unblinded fashion. Flow cytometry plots were generated with FlowJo Software and are representative samples. All the quantitative plots were generated using GraphPad Prism 8 Software for Windows (64-Bit) (San Diego, CA). For in vitro activity assays, data were analyzed using a non-linear sigmoidal, 4PL curve fit model without constraints. Statistical analysis was also performed using GraphPad Prism software (San Diego, CA). Two sample comparisons used the students t-test while comparisons of more than two groups used an analysis of variance (ANOVA) test with multiple comparisons. Antitumor effects over time were analyzed by using a mixed-effects model, whereas antitumor effects on specific times points were analyzed using an unpaired t test. For the NanoString dataset, statistical analysis was performed using nSolver™ software with the Advanced Analysis Module installed.

### 1.2 Results

### Inducible IL-2 Signaling and Activity is Dependent on Proteolytic Activation

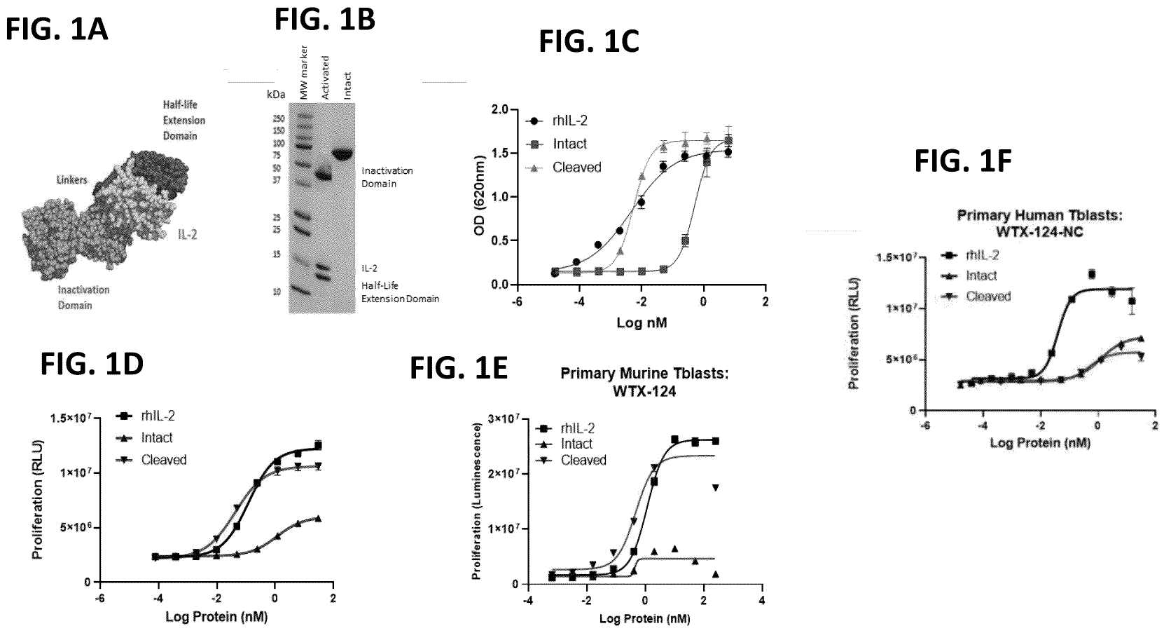

Compound 1, an inducible IL-2 prodrug, was designed to enhance the clinical profile of recombinant human IL-2 treatment by facilitating less frequent systemic delivery, increasing the tumor exposure of the molecule, and decreasing the toxicity associated with high-dose IL-2 (FIG. 1A). Compound 1 includes native human IL-2, a Fab antibody fragment that prevents IL-2 from binding to the medium affinity IL-2 receptor (IL-2Rβ/γ), thereby acting as an inactivation domain, and an anti-human serum albumin (αHSA) single domain antibody acting as a half-life extension domain. These two domains are linked to the IL-2 payload via a protease-cleavable linker sequence. Compound 1 in the prodrug state is half-life extended, and the activity of IL-2 is inhibited by blocking the binding of the molecule to the IL-2 receptors. However, when the linkers are enzymatically cleaved in tumor tissue, the result is the removal of the half-life extension and inactivation domains, and the release of native IL-2 (FIG. 1B)

In order to measure the difference in activity between the intact and protease cleaved Compound 1, HEK-Blue IL-2 reporter cells were incubated with either recombinant human IL-2 (rhIL-2), intact Compound 1, or protease activated Compound 1 (cleaved) and then IL-2 signaling was measured. In this assay, intact Compound 1 had approximately 100-fold less activity than either rhIL-2 or cleaved Compound 1 (FIG. 1C). Additionally, human PBMCs were stimulated with PHA to form Tblasts, which express the high affinity IL-2 receptor (CD25/CD122/CD132) (FIGS. 8A-8B) and respond to IL-2 signaling by proliferating. Human Tblasts from multiple donors were exposed to rhIL-2, intact Compound 1, or cleaved Compound 1 for 72 hours and then Tblast proliferation was measured. In this system, intact Compound 1 had less activity than either cleaved Compound 1 or rhIL-2 across multiple donors (FIG. 1D, FIG. 8C). More specifically, intact Compound 1 had approximately 23-fold less activity in terms of an increased EC₅₀ and plateaued at only 60% of the maximum activity seen with either cleaved Compound 1 or rhIL-2.

The activity of intact and cleaved Compound 1 was also characterized in a mouse primary T blast assay. While cleaved Compound land rhIL-2 induced similar proliferation by murine Tblasts, intact Compound 1 had almost no measurable activity in cells isolated from multiple mice (FIG. 1E, FIG. 8D). To confirm that the activity of cleaved was Compound 1 dependent on linker cleavage and not an unknown processing event, a non-cleavable variant of Compound 1, named Compound 1-NC, was generated by replacing the linker sequence with a non-cleavable glycine/serine sequence. As a control, Compound 1-NC was treated to the same enzymatic digestion as Compound 1 before being tested in human Tblasts. As expected, no difference in activity was seen between the intact and “cleaved” forms of Compound 1-NC, demonstrating the necessity for linker cleavage to restore full activity of IL-2 released from the Compound 1 prodrug (FIG. 1F)

### Compound 1 Treatment Controlled Tumor Growth in a Cleavage-Dependent Manner

To test whether Compound 1 treatment could inhibit tumor growth, mice were implanted with MC38 tumor cells and randomized into treatment groups when the tumors were between 100-150 mm³. Mice were then treated twice a week with vehicle (PBS) or titrated amounts of either Compound 1 or Compound 1-NC (non-cleavable control) for a total of four doses. Given the residual activity observed with intact Compound 1 when tested at a high concentration in vitro, Compound 1-NC also acts as a control for the level of the in vivo activity derived specifically from intact Compound 1 (FIGS. 1C-1E). In this model, even the lowest dose of Compound 1 (25 μg) resulted in statistically significant tumor growth inhibition (FIGS. 2A, 2I and 13B). Furthermore, doses of 100 μg, 150 μg, or 300 μg were all highly efficacious. Of the 24 mice in those dosing groups, 23 had complete responses, with no measurable tumor remaining at the end of the experiment (FIG. 2A). All of these dose levels were well tolerated by the mice, with no signs of body weight loss (FIG. 9A). In contrast, even when Compound 1-NC was dosed at the highest tested amount (300 μg), it had negligible effects on tumor growth, demonstrating that the anti-tumor activity of Compound 1 is dependent on in vivo enzymatic cleavage of its linkers. Further, anti-tumor activity of Compound 1 was decreased when CD8+ T cells were depleted (FIG. 2I), demonstrating that Compound 1 supports the host effector cell anti-tumor response.

The major impediment to widespread clinical use of recombinant human IL-2 is the toxicity observed when this cytokine is given systemically. Since Compound 1 was designed to enhance the PK profile of IL-2 treatment, it was possible that using a half-life extended IL-2 could actually result in even greater toxicity than the original free cytokine. Therefore, we tested whether the half-life extension element of the Compound 1 was required for the anti-tumor activity. Indeed, when an inducible IL-2 prodrug lacking the half-life extension element (WW0057) was tested in vivo, this molecule failed to generate anti-tumor immunity, even when given at 10× the fully efficacious dose of Compound 1(FIG. 13C), demonstrating the necessity of including the half-life extension element to generate anti-tumor immunity with an IL-2 prodrug. We examined whether or not the blocking element of Compound 1 was able to prevent the increased toxicity that would be expected with exposure to half-life extended IL-2.

To better understand the effectiveness of the inactivation domain at limiting toxicity, a variant of Compound 1 without the blocking domain was created (WWO177). WWO177 differs from Compound 1 in that it contains a non-cleavable linker sequence between the half-life extension domain and the fully active IL-2, and it does not have an inactivation domain, thereby representing the level of toxicity that should be expected if the inactivation domain was not functioning properly. MC38 tumor-bearing mice were dosed with either WWO177 or Compound 1, and their weight was monitored over time (FIG. 2B). After only 2 doses of WWO177, dosing had to be halted due to body weight loss, and only 2/7 mice survived. In contrast, mice treated with four doses of Compound 1 had no evidence of weight loss, despite being given approximately 26-times the molar amount of IL-2 that was administered to the group receiving WW0177.

While loss is a useful surrogate to monitor overall toxicity in regard to immunotherapy, it was also important to investigate the effects of Compound 1 on organ specific toxicity. Vascular leak syndrome (VLS) is the major dose-limiting toxicity associated with high dose IL-2 treatment in the clinic, and it not only restricts the clinical utility of high dose IL-2, but also prevents half-life extended IL-2 from being a viable clinical strategy. In mice, VLS is induced by high doses of recombinant IL-2 and can be measured by examining the amount of Evan's Blue dye that leaks into the lungs following i.v. injection. In agreement with the overall toxicity data, when recombinant human IL-2, WW0177, or Compound 1 were administered in equimolar amounts, only recombinant human IL-2 and WWO177 resulted on detectable levels of Evans Blue leaking into the lungs, while the Compound 1 did not (FIG. 13D). These data demonstrate that the blocking domain of Compound 1 effectively blocks the activity of IL-2 in peripheral tissues and prevents the induction of VLS compared to peripherally active IL-2 molecules.

Although the inactivation domain of Compound 1 is highly effective, the activity of this domain depends on the blocker remaining linked to the IL-2 molecule (FIG. 1) To examine the stability of Compound 1 in the periphery, Compound 1 was incubated in murine plasma from either naïve or MC38 tumor-bearing mice for 24, 48, or 72 hours before the level of intact Compound 1 was measured by western blot. In agreement with the tolerability of the molecule, there was no evidence of Compound 1 cleavage across all the tested timepoints (FIG. 2C).

In addition to managing peripheral toxicity, Compound 1 was designed to facilitate less frequent, systemic delivery of the treatment without sacrificing potency and anti-tumor activity of high dose IL-2. Therefore, it was important to directly compare the activity of Compound 1 to native IL-2. MC38 tumor-bearing mice were treated with titrated amounts of either Compound 1 as before (twice weekly for two weeks), or rhIL-2 dosed twice a day for two weeks (dosing regimen: 5 days dosing, 2 days rest schedule for 2 weeks). The differences in the dosing schedules reflects the poor in vivo pharmacokinetic properties of rhIL-2 in both humans (15) and mice (16), and mimics the dosing of patients with high-dose IL-2 in the clinic. Since the two treatments are delivered on different dosing regimens, the correct way to compare treatment groups is to compare the total amount of IL-2 delivered during the dosing period. When MC38 tumor-bearing mice were treated with a total of 5.04 μM of Compound 1, complete tumor rejection was seen in 8/8 mice. In contrast, even when mice were treated with 15.5 μM of native IL-2 (three times the total amount of IL-2 dosed with Compound 1) only 5/8 mice completely rejected the tumors (FIG. 2D).

As noted previously, the poor pharmacokinetic profile (t_{1/2}<1 hour) of proleukin treatment results in an impractical dosing regimen, with many patients receiving a high dose every 8 hours for up to 15 doses (17). Likewise, in mice, rhIL-2 is rapidly cleared from circulation (16). We hypothesized that the increased activity of Compound 1 compared with native IL-2 is due to its extended half-life and pharmacokinetic profile. To confirm this, tumor-bearing mice were dosed once on Day 0 and once on Day 4, and the drug exposure was measured in the plasma and within the tumor at various timepoints. In contrast to rhIL-2, Compound 1 dosing resulted in extended exposure in the plasma, with a half-life of approximately 20 hours, and exposure maintained over the course of 4 days (FIG. 2E). Additionally, intraperitoneal dosing of Compound 1 resulted in prolonged drug exposure within the tumor itself, demonstrating tissue penetrance by Compound 1 (FIG. 2F). Total Compound 1 levels reached a Cₘₐₓ at 6 hours post-dosing in the plasma and peaked at 12 hours post-dosing in the tumor.

Compound 1 was designed to restrict the systemic activity of IL-2 while delivering fully active IL-2 locally to the tumor via the use of cleavable linkers. To test whether systemic dosing of Compound 1 resulted in localized delivery of rhIL-2 into the tumor, plasma and tumor samples were collected at various timepoints after dosing and analyzed for the presence of free human IL-2 (i.e. not bound to the blocking Fab) released due to the enzymatic processing. To specifically measure human IL-2 released from the IL-2 prodrug by proteolytic processing, we identified an ELISA kit that was specific for human IL-2 (FIG. 13E), where the blocking domain of Compound 1 prevented the binding of the ELISA detection reagents (FIG. 13F). This allowed us to assess the level of free, human IL-2, which could only be present in the murine system through the in vivo processing of Compound 1. Systemic dosing with Compound 1 resulted in almost no detectable free IL-2 in the plasma (FIG. 2G). In contrast, systemic dosing with Compound 1 did result in prolonged levels of detectable free IL-2 in the tumor (FIG. 211), demonstrating that tumor dependent processing drives increased exposure of fully active IL-2 in the tumor following systemic delivery of the Compound 1.

To better quantify the differences between plasma and tumor in terms of Compound 1 processing, the area under the exposure curves (FIGS. 2E-2H) was measured and is reported in Table 3. Strikingly, while the amount of total Compound 1 in the plasma was about 18-fold greater than what was detected in the tumor, the amount of free IL-2 in the tumor was still over 5-fold greater than what was detected in the plasma, which suggests an approximately 93-fold increase in Compound 1 processing in the tumor compared with the plasma.

TABLE 3
Area under the curve analysis of total Compound 1 and free
IL-2 in the plasma and tumors of MC38 tumor-bearing mice.

| | | PLASMA | TUMOR | TUMOR/PLASMA |
|---|---|---|---|---|
| TARGET | MEASUREMENTS | EXPOSURE | EXPOSURE | EXPOSURE |
| READOUT | INCLUDED | (NG*H*L−1) | (NG*H*L⁻¹) | RATIO |
| Total IL-2 | Intact WTX-124 + | 22,438 | 1,283 | 0.057 |
| | free human IL-2 | | | |
| Free IL-2 | Free human IL-2 | 3.411 | 18.12 | 5.31 |
| | only | | | |

*AUC measurements were calculated from the plots in FIGS. 2E-2H using prism software. These values were used to calculate the exposure ratio comparing the exposure in the tumor to the exposure in the plasma.
*AUC, area under the curve; IL-2, interleukin-2; MC, murine colon.

The therapeutic window (TW) of a therapy is defined as the ratio of the maximum tolerated dose and the lowest efficacious dose, thereby identifying the difference between activity and serious adverse events. In the clinic, the TW for proleukin is relatively small. Similarly, the TW of rhIL-2 in MC8 tumor bearing mice was calculated to be less than 4-fold in our model (FIG. 9B). Since the half-life extension element of WWO177 makes it a more active version of IL-2, less WWO177 is required to reach full efficacy compared to recombinant hIL-2. However, WWO177 also has a lower maximum tolerated dose, resulting in a TW of less than 2. However, since Compound 1 was significantly more active than equimolar amounts of rhIL-2, and no toxicity was seen in tumor bearing mice dosed with up to 960 μg/dose of Compound 1, the TW of Compound 1 is greater than 20 at a minimum, representing at least a 5-fold improvement over rhIL-2 (FIG. 13G). These data demonstrate that Compound 1 is not simply an attenuated IL-2 molecule, but instead a unique, inducible IL-2 pro-drug that enhances the activity of the payload while restricting its systemic activity.

### Compound 1 Treatment of MC38 Tumor-Bearing Mice Resulted in Immunological Memory

One hallmark of immunological rejection of a tumor is the development of protective memory against subsequent tumor re-challenge. To test whether Compound 1 treatment resulted in tumor-specific memory following tumor rejection, mice were implanted with MC38 tumor cells and randomized into vehicle or Compound 1 treatment groups, and tumor growth was measured. As with previous studies, Compound 1 treatment resulted in tumor rejection, whereas the control tumors continued to grow.

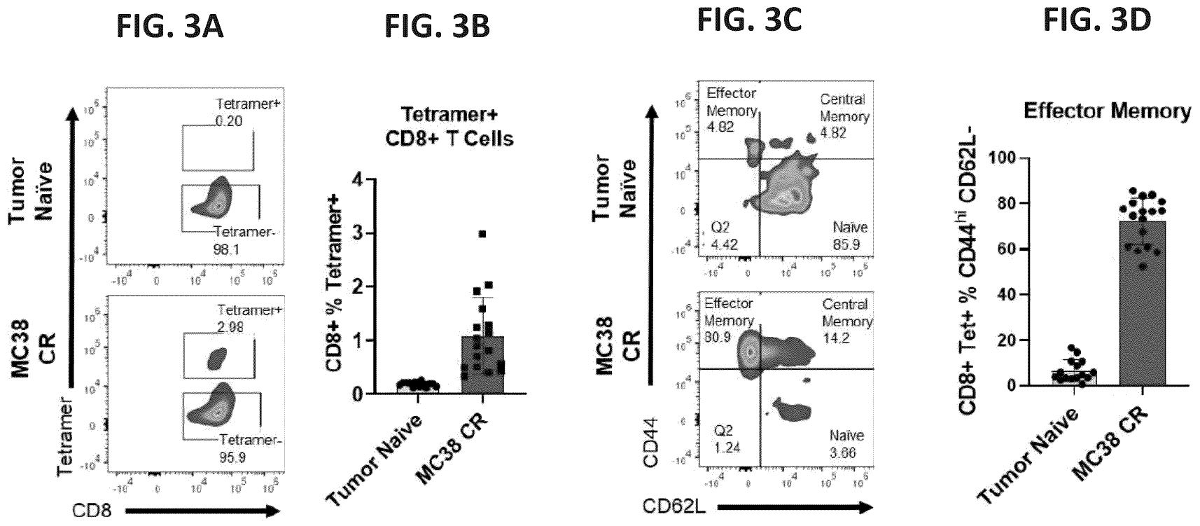

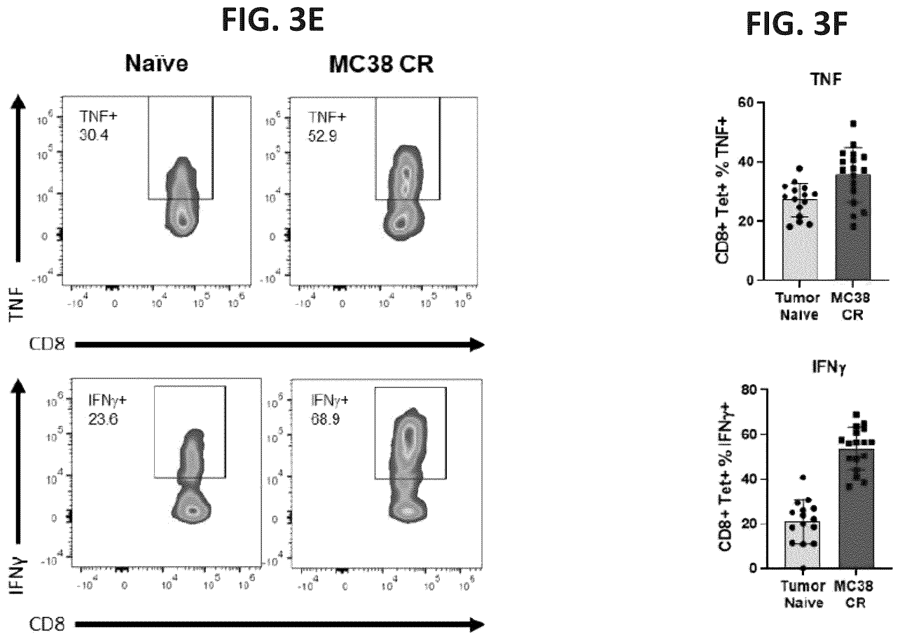

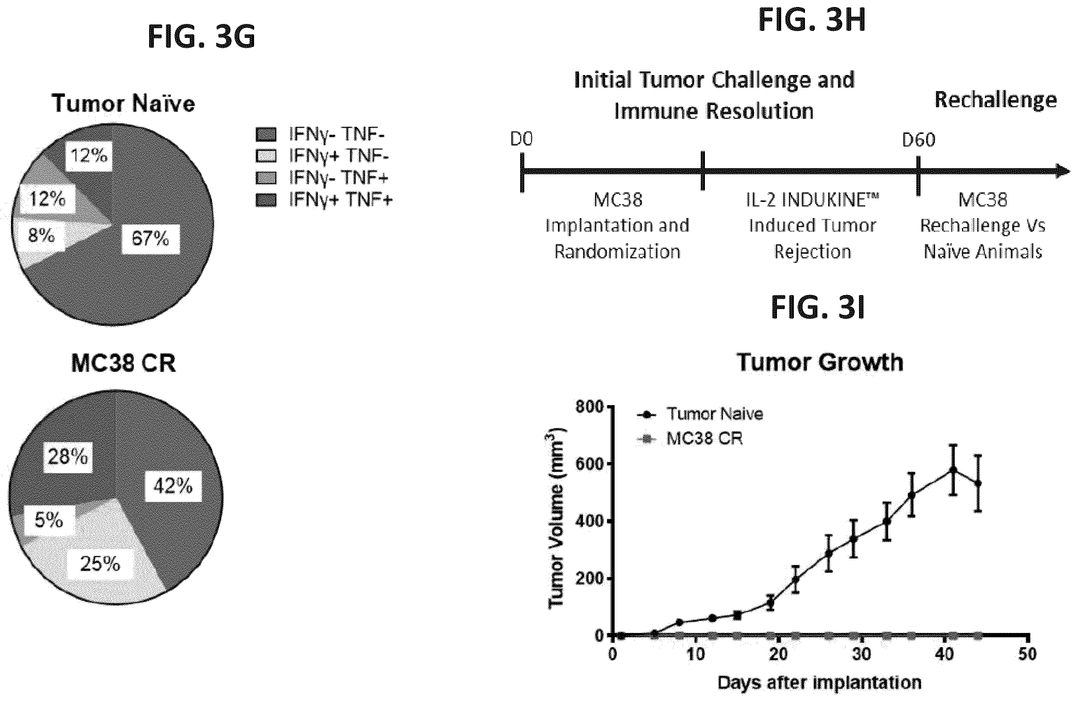

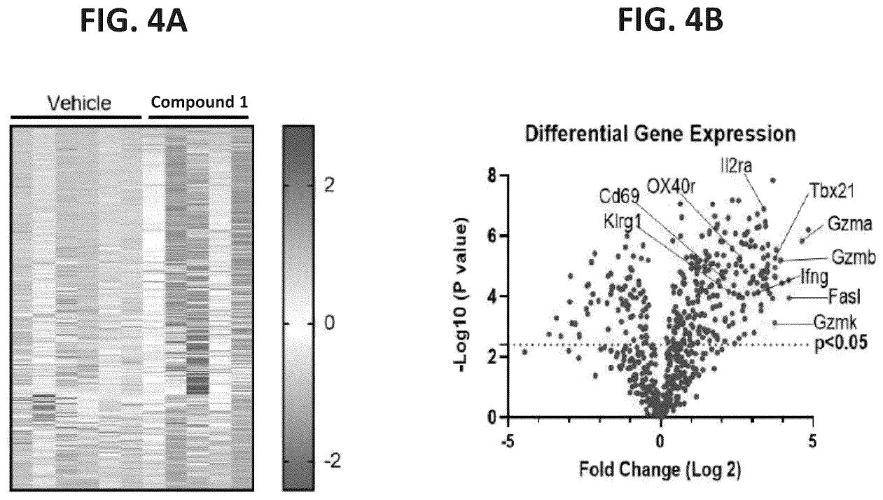

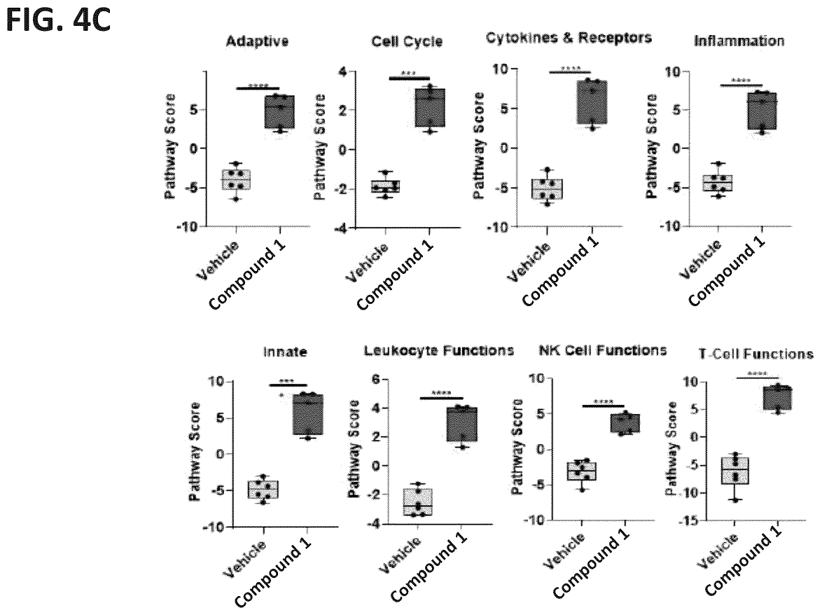

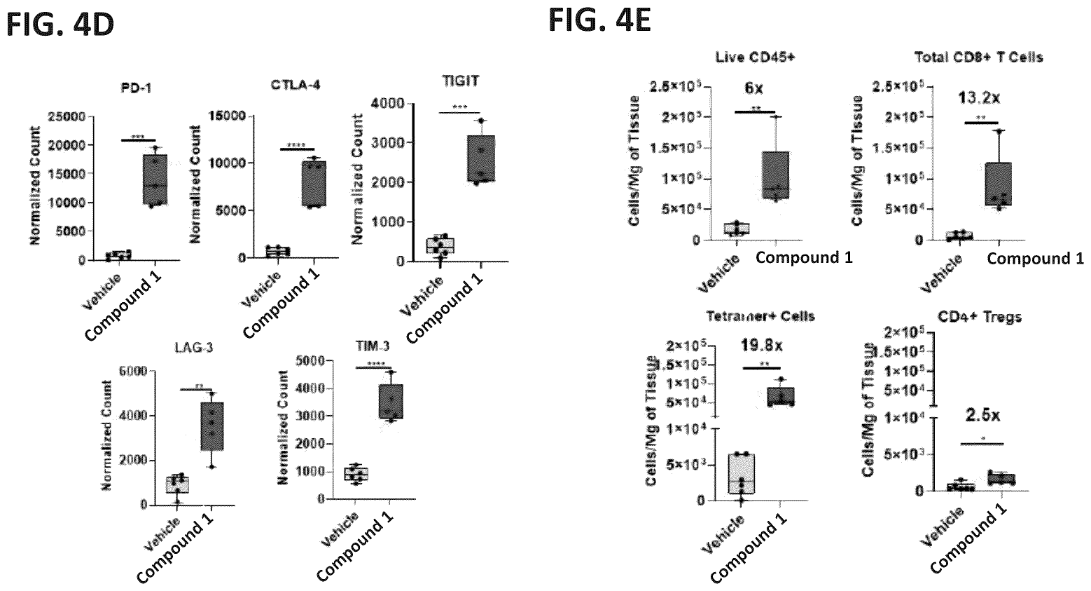

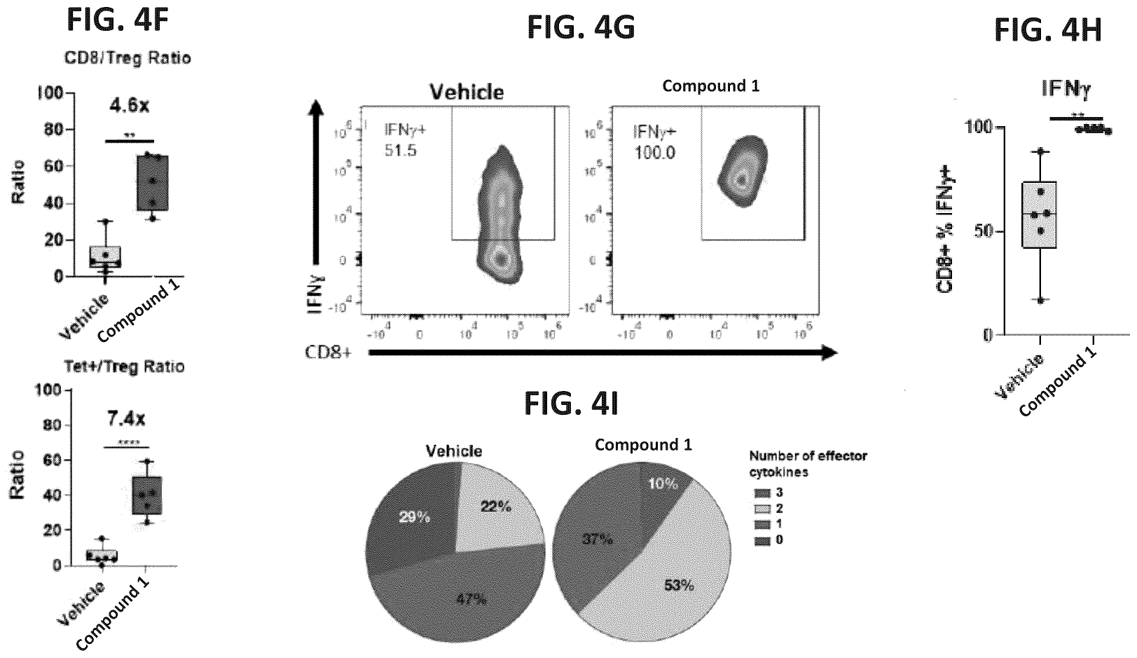

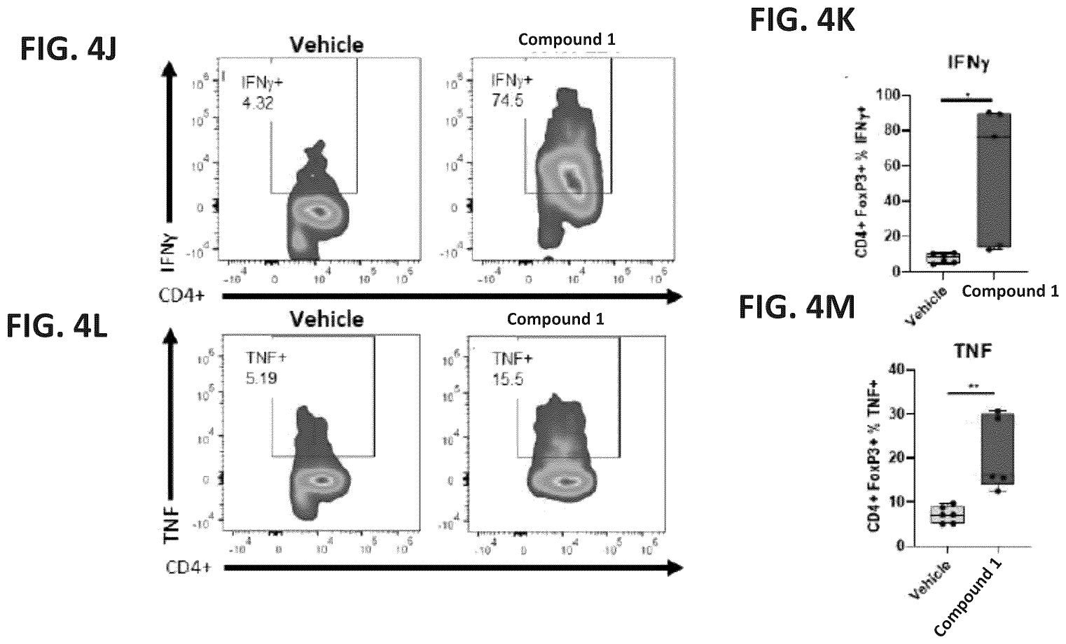

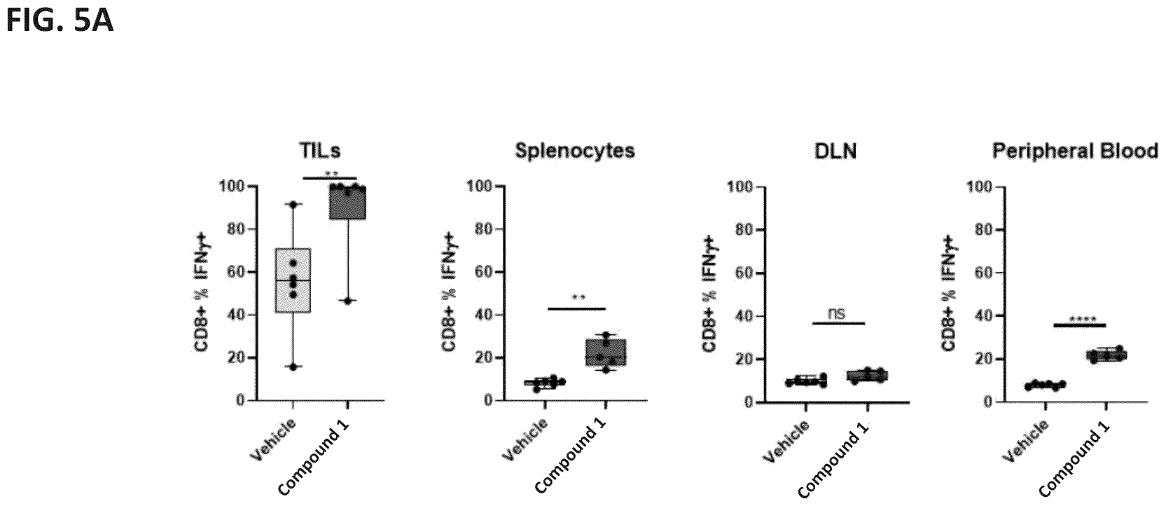

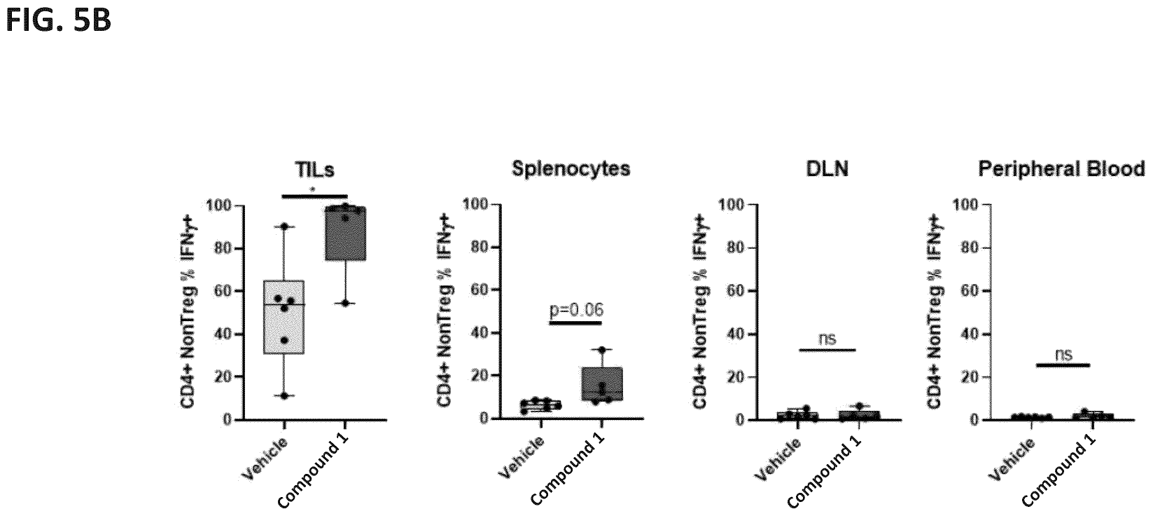

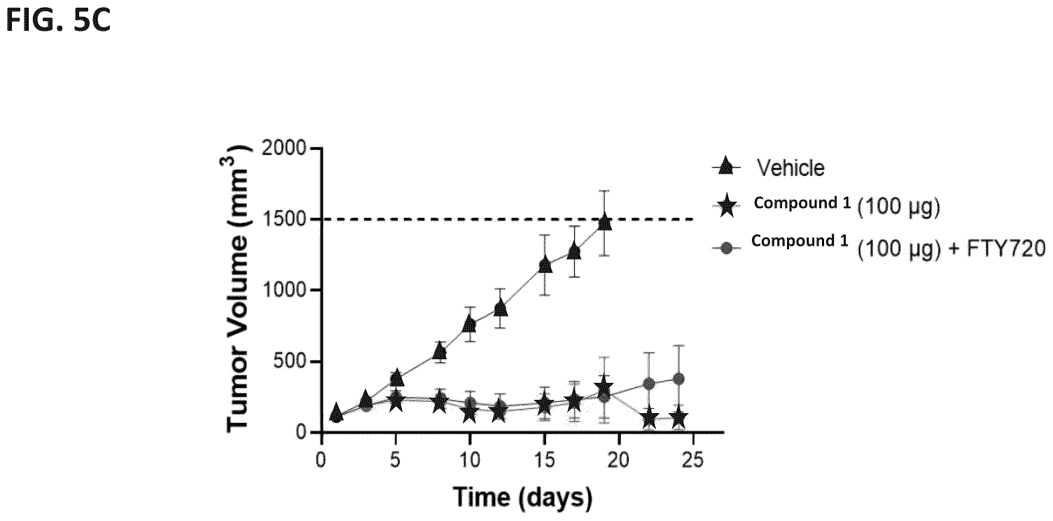

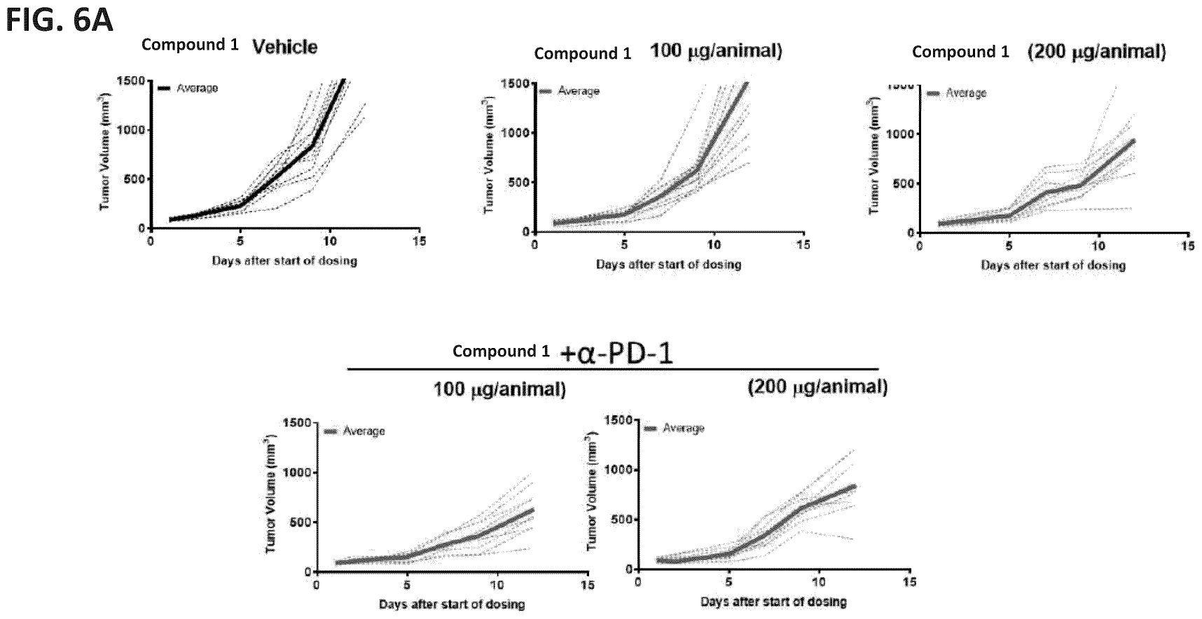

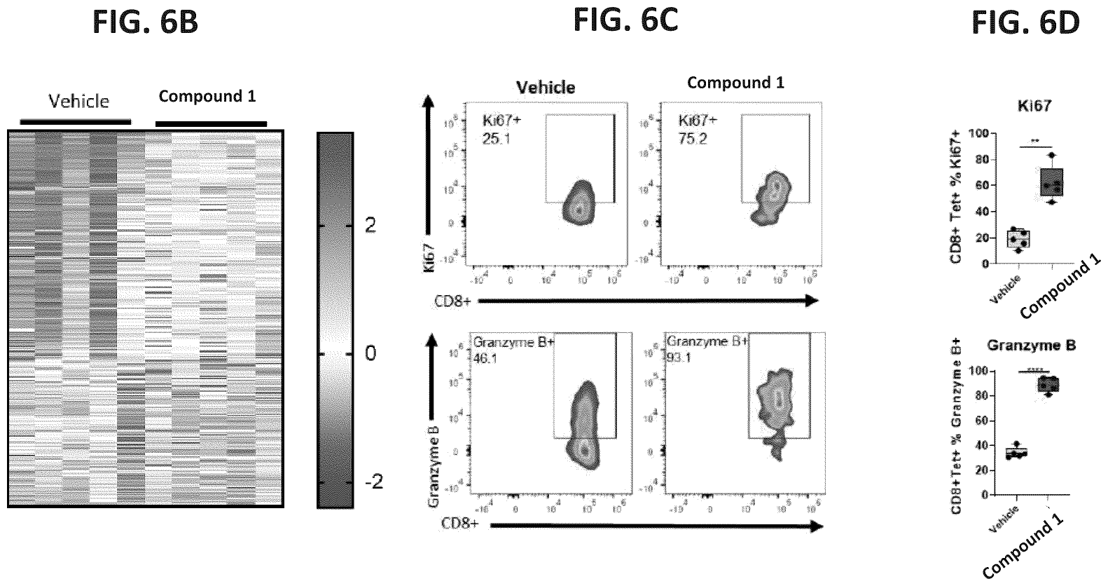

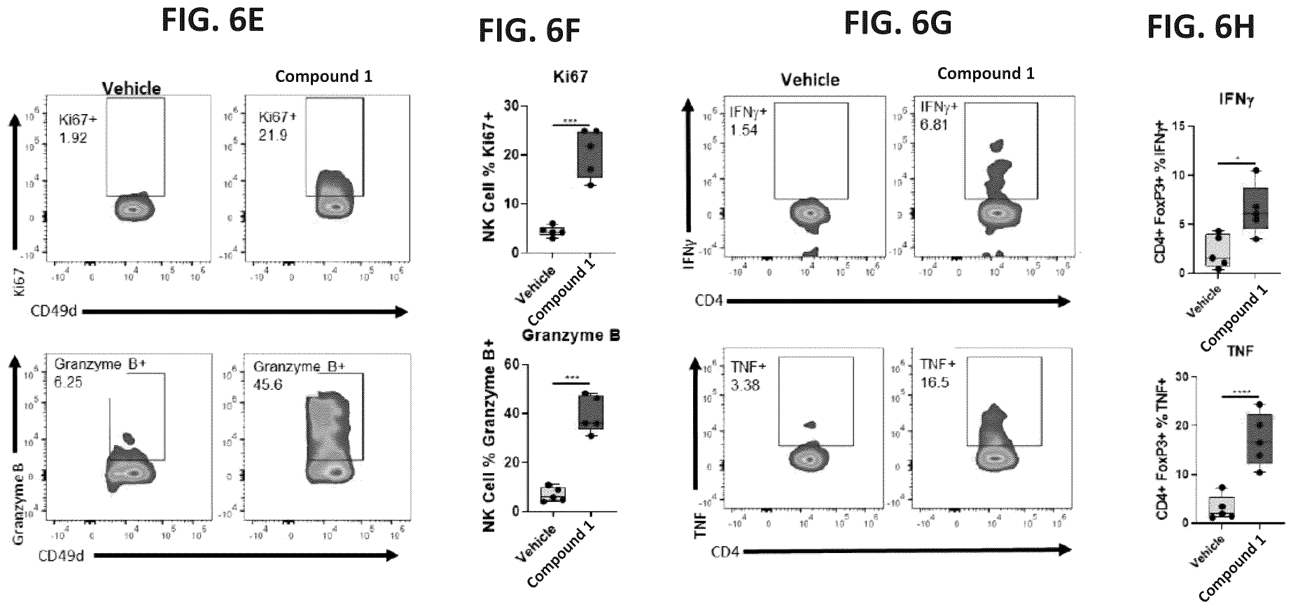

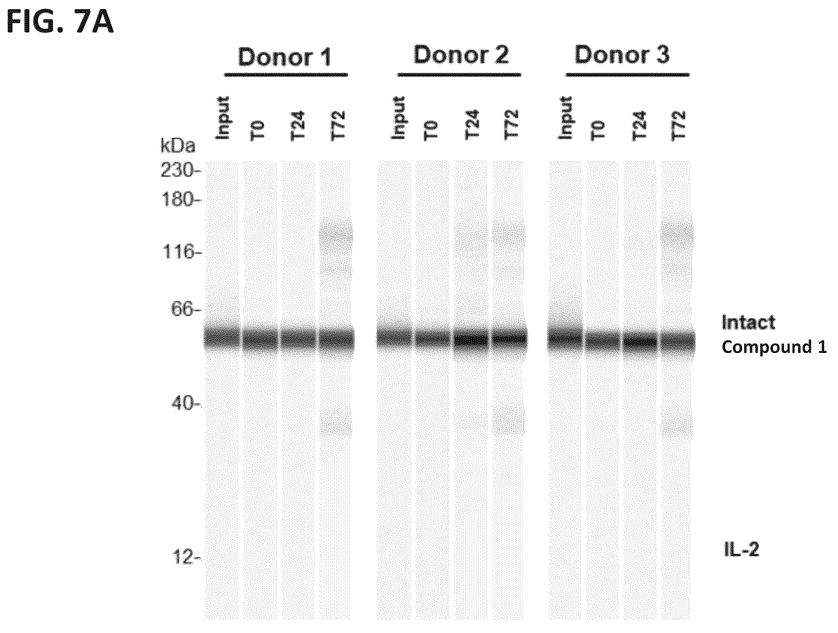

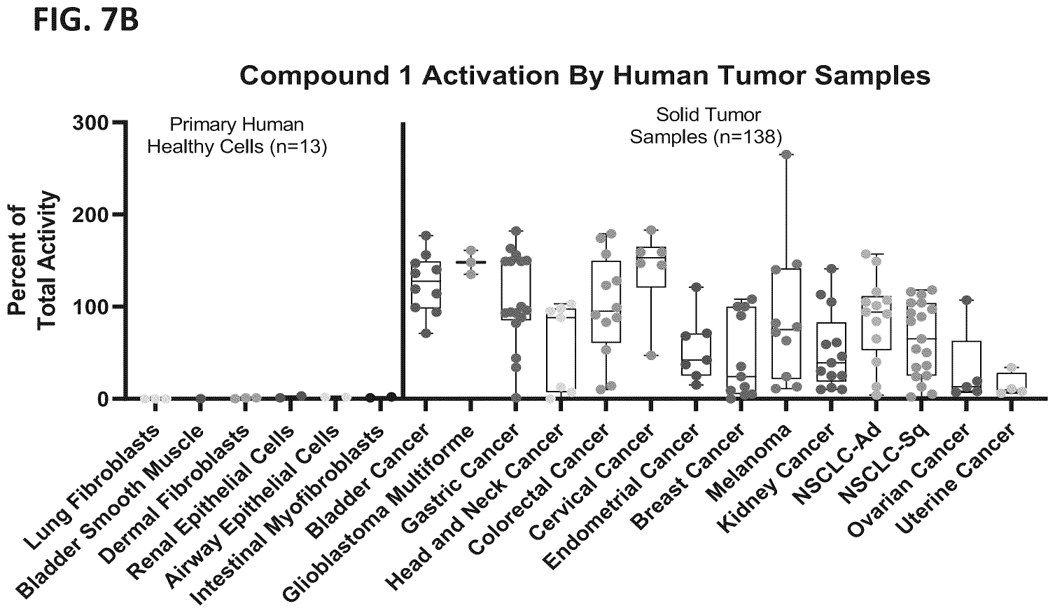

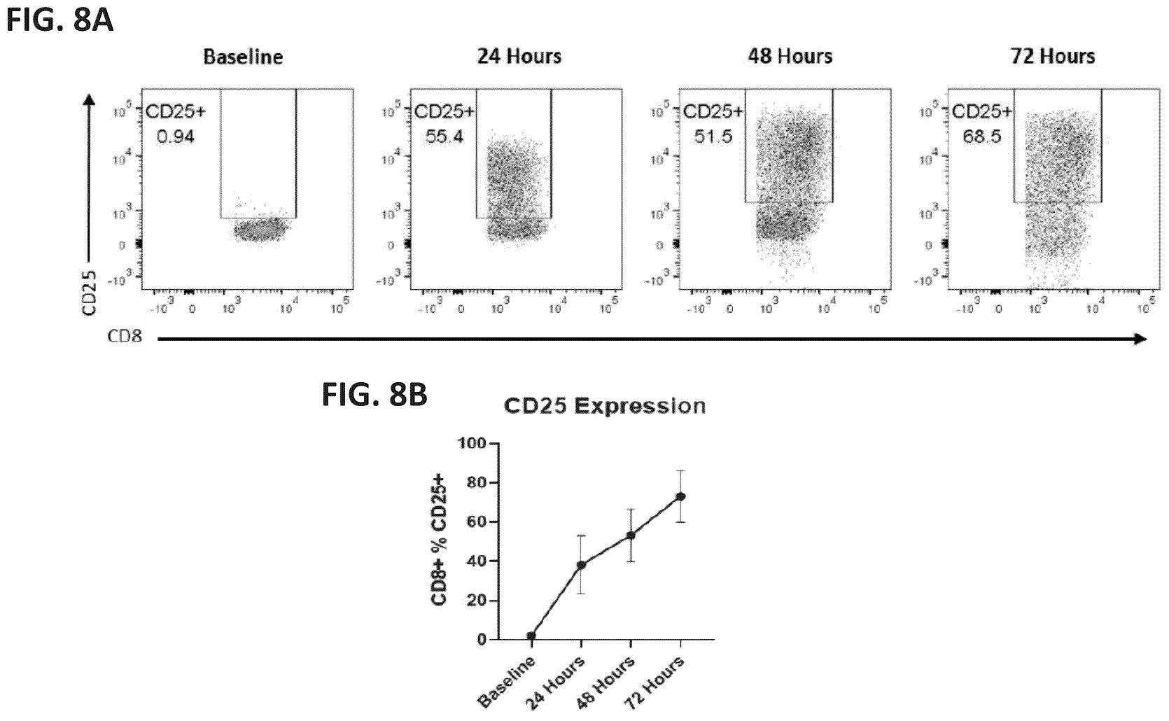

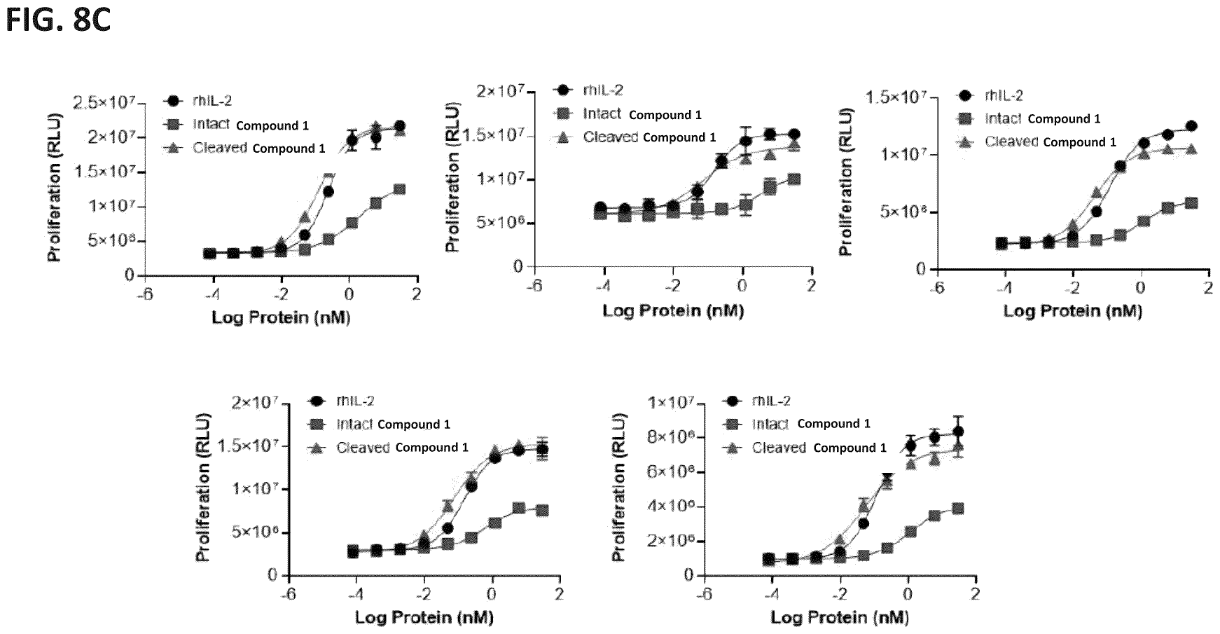

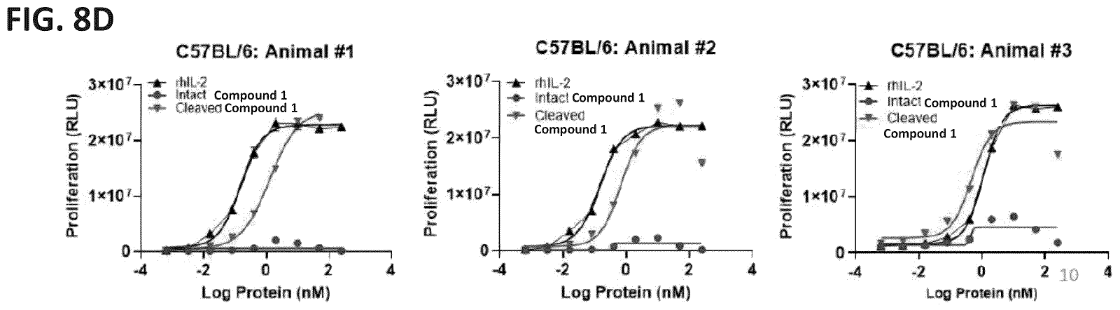

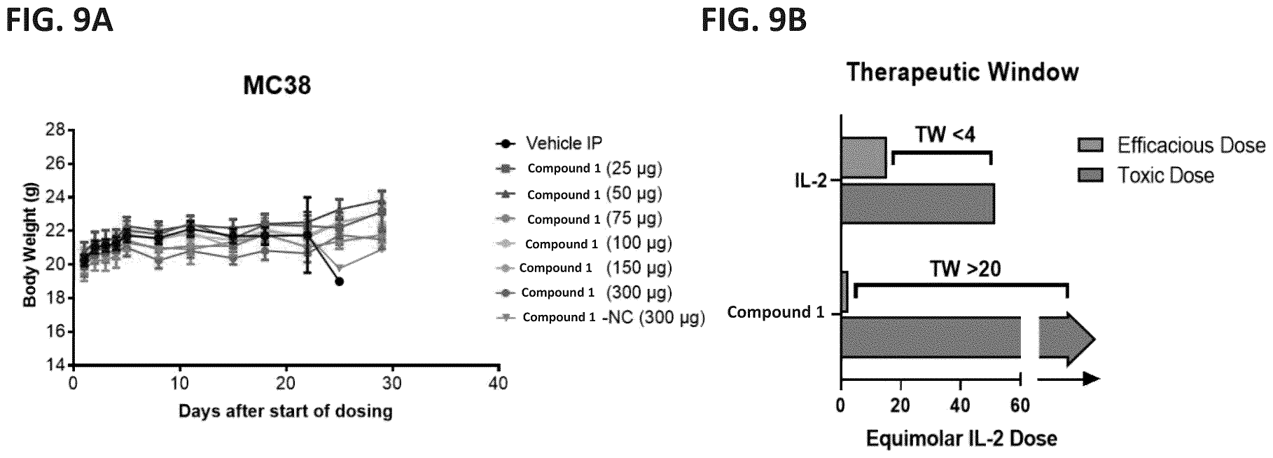

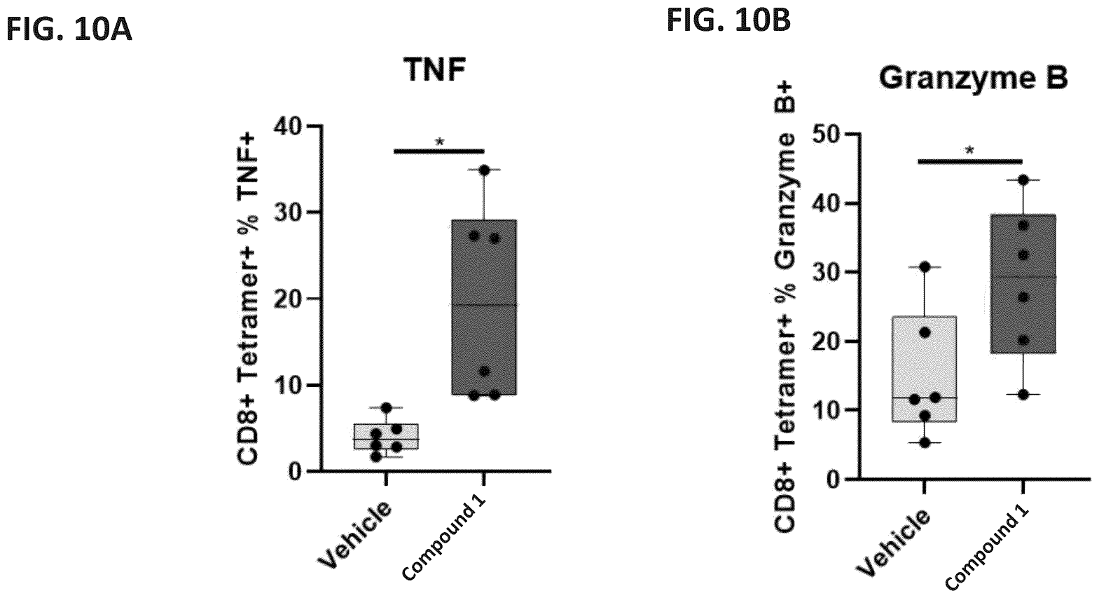

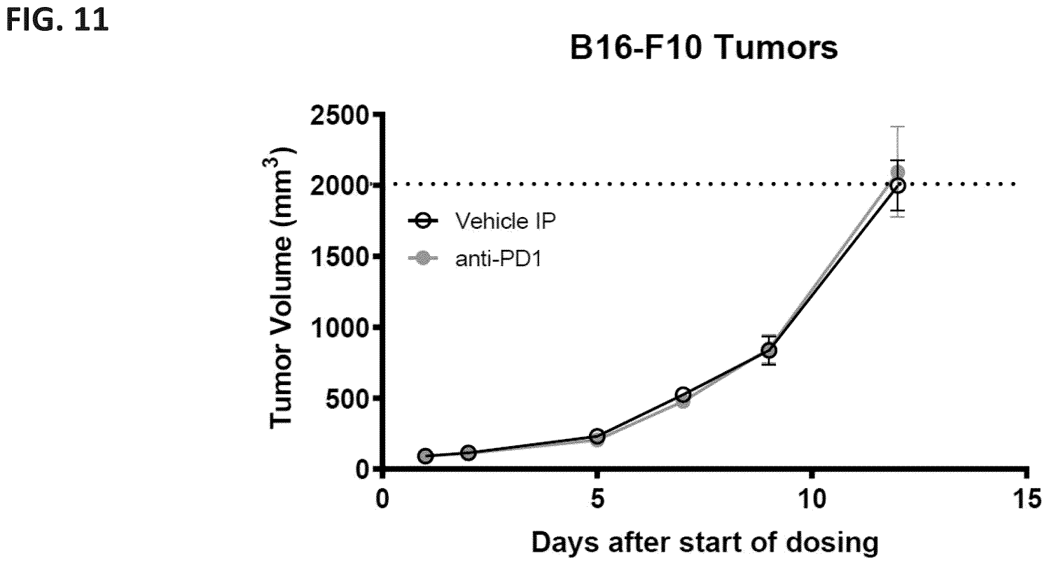

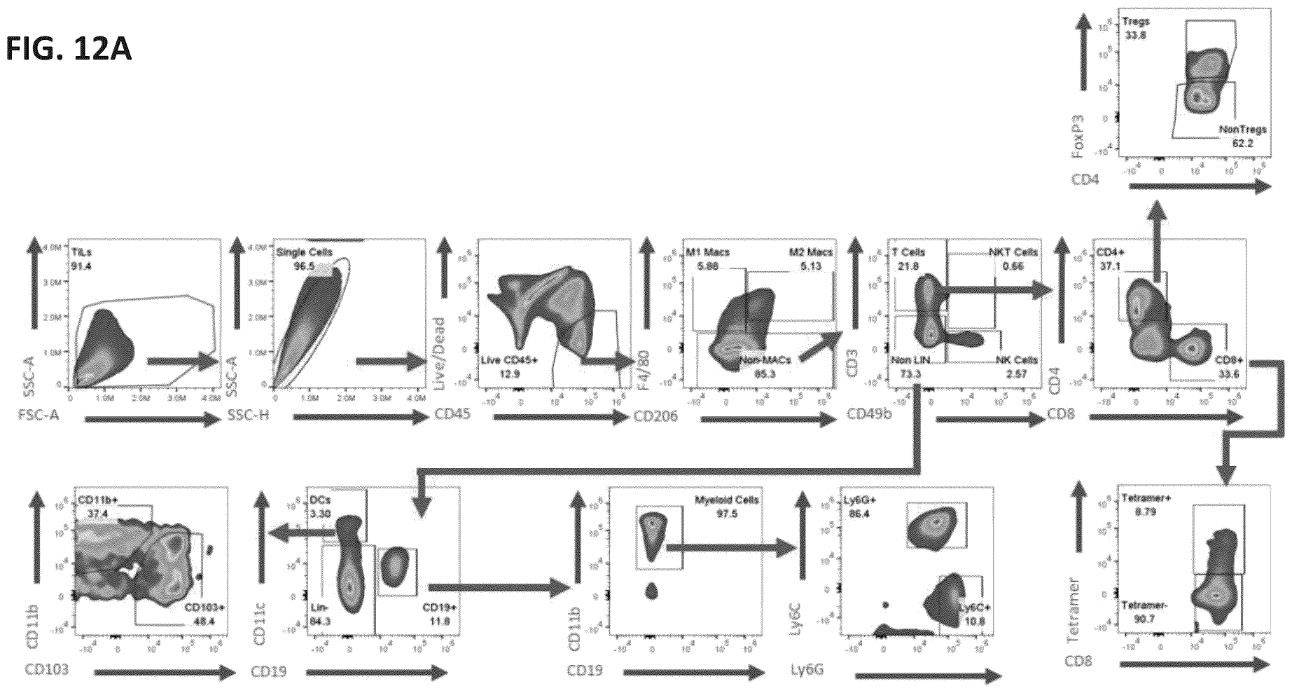

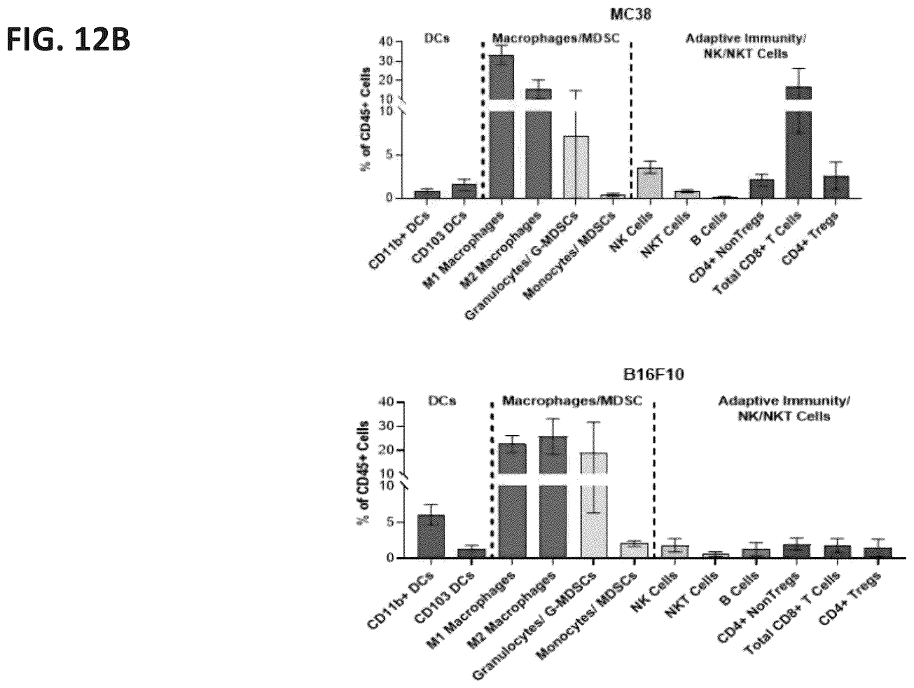

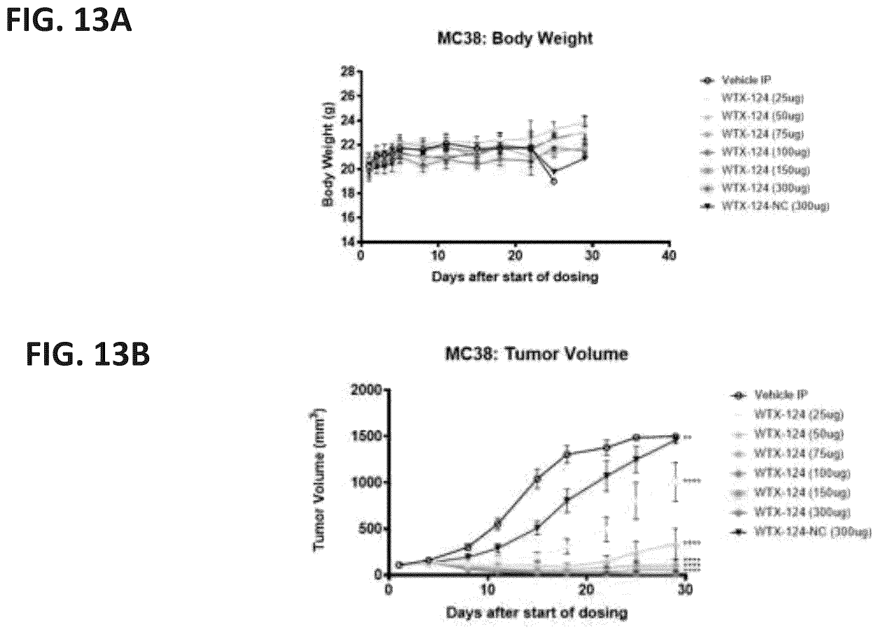

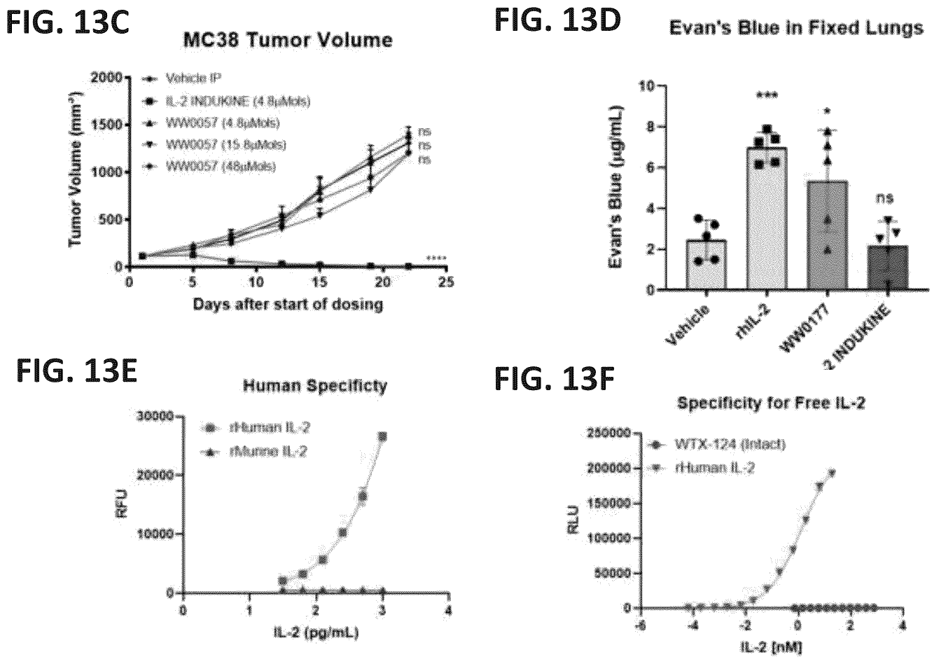

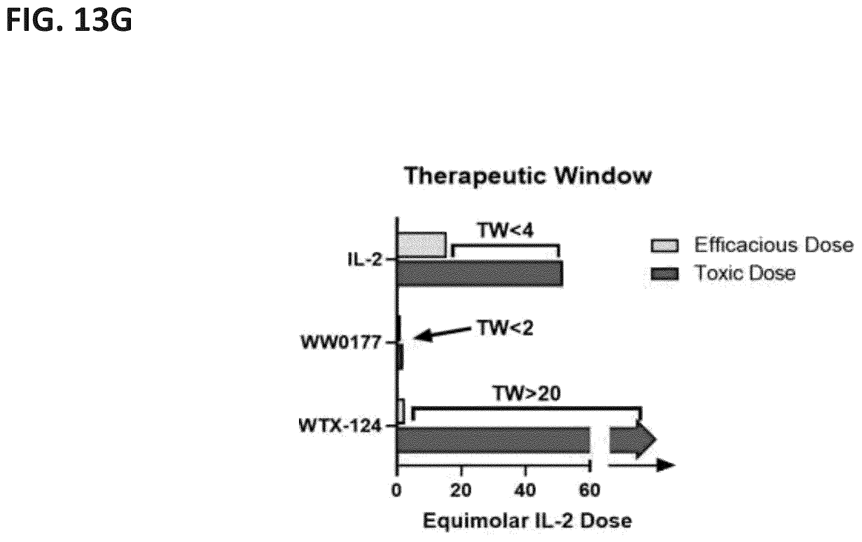

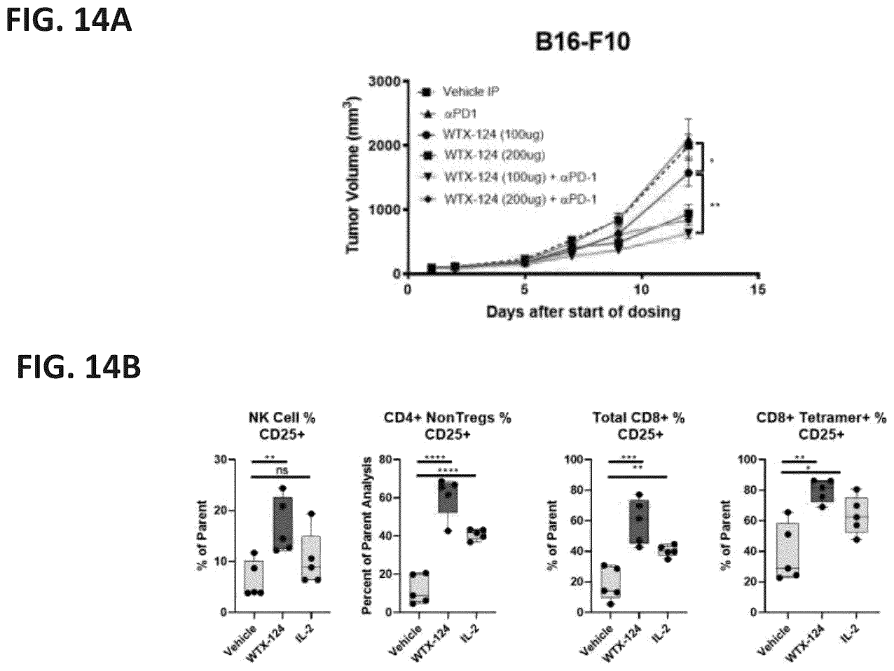

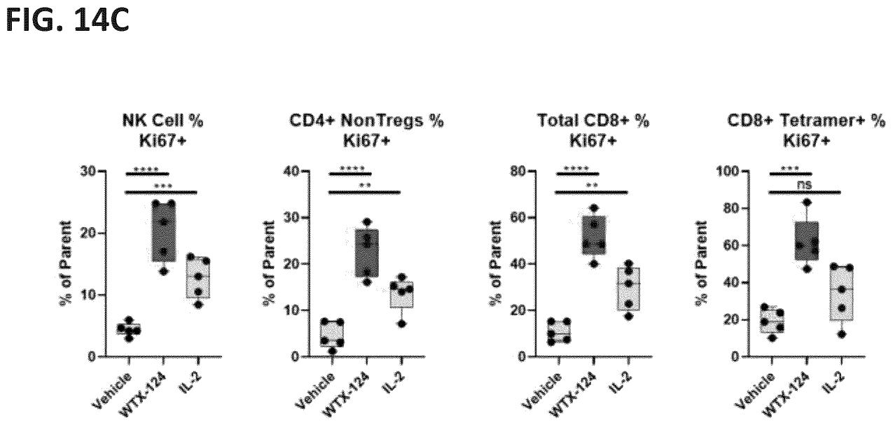

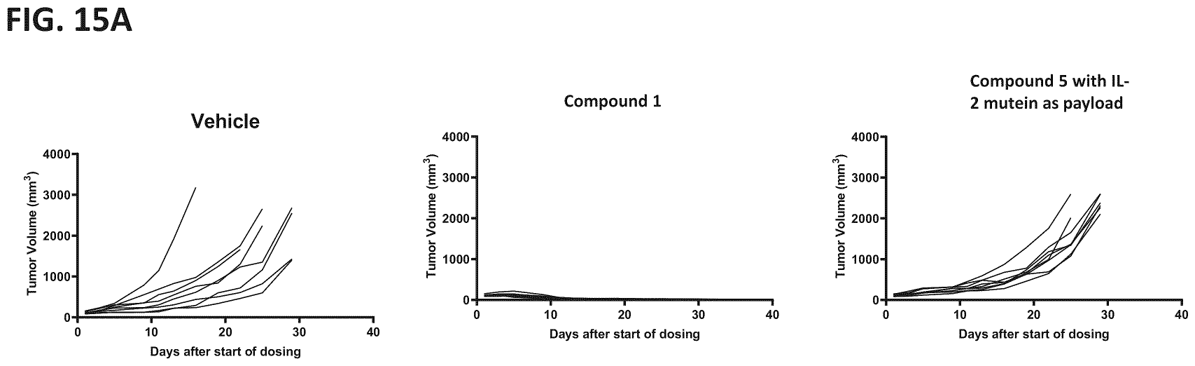

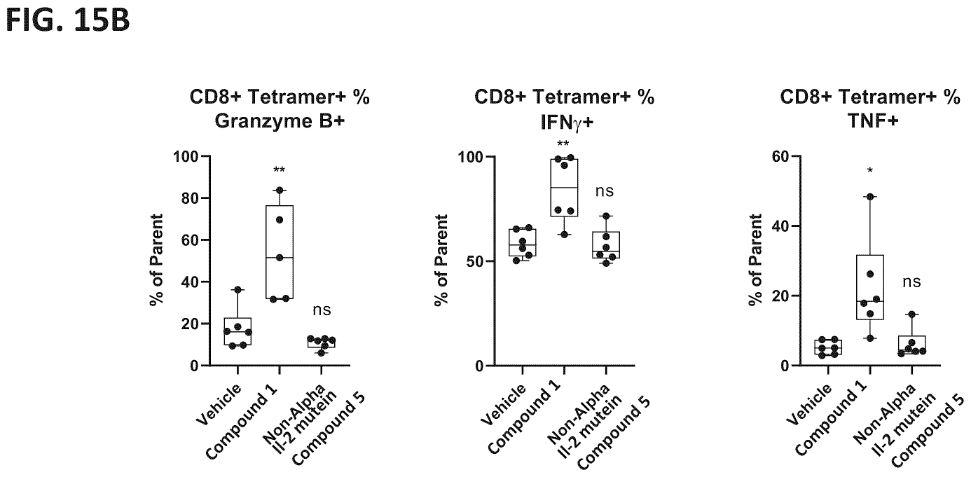

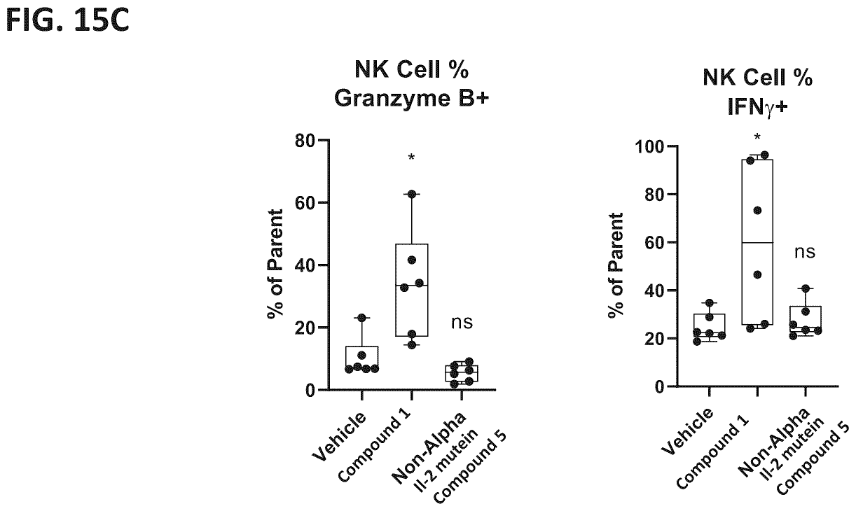

To examine whether tumor rejection in Compound 1-treated mice resulted in immunological memory, spleens from mice were examined for the presence of tumor-specific memory CD8+ T cells 6 months after the initial MC38 implantation (MC38 CR mice) (FIG. 3A). Previous studies have identified that a peptide derived from Murine Leukemia Virus protein gp70 (KSPWFTTL) (SEQ ID NO: 7) is an antigen presented by MC38 tumors and that T cells specific for this antigen can be identified using fluorescently labeled MHC peptide complexes known as tetramers (18). Spleens from MC38 CR mice had a higher overall frequency of tetramer-positive CD8+ T cells than age matched tumor-naïve mice (FIGS. 3A-3B). Furthermore, although the tetramer-positive cells from tumor-naïve mice largely maintained a naïve phenotype, cells from MC38 CR mice were predominantly of an effector memory phenotype (CD44^{hi}CD62^{low}) (FIGS. 3C-3D). Furthermore, upon re-stimulation, tetramer-positive cells from MC38 CR mice secreted more of the effector cytokines TNF and IFNγ (FIGS. 3E-3F). Co-expression of two or more effector cytokines is known as polyfunctionality, and it is associated with greater cytolytic activity in T cells (19). WTX-124 treatment also significantly increased the frequency of polyfunctional T cells following re-stimulation (FIG. 3G). These data are consistent with the idea that Compound 1 treatment results in immune mediated tumor rejection which then translates into immunological memory.

Although the phenotype of these splenocytes suggests the generation of tumor-specific memory, the ultimate test of a memory response is protection against rechallenge. Therefore, Compound 1-induced MC38 CR mice were re-challenged with MC38 tumor cells 60 days after the initial implantation (FIG. 311). Importantly, no treatment was administered during the rechallenge. Unlike the tumor-naïve mice, none of the MC38 CR mice developed tumors (FIG. 3I), demonstrating that inducible IL-2 protein treatment-induced tumor rejection results in immunological memory and protection against subsequent tumor rechallenge.

### Compound 1 Treatment Amplified MC38 Tumor Infiltration and Induced Immune Cell Activation

To better understand the mechanism by which Compound 1 treatment induces anti-tumor immunity, MC38 tumor-bearing mice were randomized into treatment groups on Day 0 and treated with either vehicle or Compound ion Day 1 and Day 4. Tumors were harvested 24 hours after their second dose. Total RNA was extracted from the single-cell suspensions and analyzed using the NanoString nCounter® PanCancer Mouse Immune Profiling Panel. Compound 1 treatment resulted in a clear shift in the transcriptional profile, with 437/770 genes in the panel having statistically significant differences in expression compared with the control group (FIG. 4A-4B). NanoString nSolver™ pathway analysis of this dataset revealed a series of immune activation-related pathways that were upregulated by Compound 1 treatment, including both broad immune activation signatures such as adaptive immunity and inflammation, as well as more specific signatures such as leukocyte function, NK cell function, and T cell function (FIG. 4C). Interestingly, the expression of several transcripts associated with immune checkpoint proteins also increased following Compound 1 treatment, including PD-1, TIGIT, and CTLA-4 (FIG. 4D). This likely reflects the overall increase in immune cell activation among the tumor-infiltrating lymphocytes (TILs), as many checkpoint proteins are upregulated during a typical immune response.

In addition to the NanoString analysis, immune cell profiling by flow cytometry was also performed. As soon as 5 days after the initial dose, Compound 1 treatment resulted in a large increase in the density of infiltrating immune cells, including tumor-specific tetramer-positive CD8+ T cells (˜19.8-fold increase) and to a lesser extent Tregs (˜2.5-fold increase) (FIG. 4E). The effect of IL-2-based therapies on Tregs is a major topic of discussion in the scientific community, as there is some concern that IL-2 treatment will result in counterproductive Treg expansion, which may hinder immunotherapy in the clinic. This has led to the development of several IL-2 variant molecules that are engineered to avoid Treg engagement. In contrast, Compound 1 is not specifically engineered to avoid Tregs, as we hypothesize that the activity of fully active IL-2 on the cytolytic cells will overcome any possible Treg activation associated with the therapy. In support of this hypothesis, the increase in tetramer-positive CD8+ T cells following Compound 1 treatment far exceeded the increase in Tregs, resulting in a significant increase in the tetramer-positive CD8+/Treg ratio after Compound 1 treatment (FIG. 4F). This finding, coupled with the extremely potent anti-tumor activity generated by Compound 1 suggests that Treg activation does not significantly impair the efficacy of Compound 1.

To assess the activation state of the tumor-infiltrating T cells, samples from the TILS were re-stimulated, and the production of IFNγ, TNF, and granzyme B was assessed. Compound 1 treatment significantly increased the frequency of tetramer-positive CD8 T cells producing IFNγ (FIGS. 4G-4H), TNF (FIG. 10A), and granzyme B (FIG. 10B). Compound 1 treatment also significantly increased the polyfunctionality of the tetramer-positive CD8+ T cells, with a greater frequency of tetramer-positive CD8+ T cells producing either two or all three of these effector cytokines compared with the control group (FIG. 4I).

Recent data have demonstrated that under certain circumstances, Tregs can also produce effector cytokines such as TNF and IFNγ, in a phenomenon known as “Treg Fragility” (20). Importantly, the production of effector cytokines by Tregs is associated with the loss of their suppressive activity. Interestingly, although very few Tregs from the control tumors produced either IFNγ or TNF, a subpopulation of Tregs from the Compound 1-treated tumors produced both these effector cytokines (FIGS. 4J-4M). Together, these data demonstrate that Compound 1 treatment increases tumor infiltration, activates tumor-specific CD8+ T cells, and causes phenotypic instability of Tregs.

### Tumor Specific Activation of Immune Cells by Compound 1 to Generate Tumor Rejection

To confirm the effects of systemic Compound 1 treatment are selective for the tumor microenvironment, effector cytokine production by T cells derived from the tumor, spleen, peripheral blood, and draining lymph node were compared after Compound 1 treatment, using the same treatment schedule as previously described. Since the tetramer+ population is selectively enriched among CD8+ T cells within the tumor, the inclusion of these cells in the analysis could bias the comparison across different sites. Therefore, tetramer-negative CD8+ T cells were specifically examined across the various tissues. As with the previous data, Compound 1 induced a significantly higher frequency of IFNγ-producing CD8+ T cells and CD4+ non-Tregs within the tumor, compared with relatively minor levels of activity seen in the examined peripheral tissues (FIGS. 5A-5B). Together with the earlier toxicity data, these data demonstrate that Compound 1 treatment does not result in widespread, peripheral T cell activation.

While the peripheral CD8+ T cell activation seen with Compound 1 treatment was limited, it remained possible that this low level of peripheral activity was still playing a role in generating the anti-tumor immunity in this model. To test whether tumor-specific activation was sufficient to generate anti-tumor immunity, mice were implanted with MC38 tumors that grew to around 100-150 mm3 before some mice were treated with Fingolimod, or FTY720. FTY720 is a small molecule that blocks the sphingosine-1-phosphate receptors, thereby preventing lymphocyte egress from the thymus and secondary lymphoid tissues (21). Therefore, any anti-tumor activity seen in FTY720-treated mice is derived from the immune cells that have already infiltrated the tumor at the start of treatment, and not from the activation and subsequent trafficking of additional lymphocytes from secondary immune tissues. Daily FTY720 treatment had no effect on the anti-tumor activity of Compound 1 (FIG. 5C) demonstrating that the activation of the TILs alone was sufficient to reject MC38 tumors. Together, these data demonstrate that systemic administration of Compound 1 to tumor-bearing animals results in tumor processing of the inducible IL-2 molecule and preferential activation of tumor-infiltrating immune cells that is sufficient to generate potent anti-tumor immunity.

In cancer patients, the presence of a pre-existing TIL population, termed a “hot” tumor, correlates with responses to immunotherapy, and the lack of a pre-existing TIL population, known as a “cold” tumor, has the opposite correlation. Murine syngeneic tumor models vary in their baseline immune infiltration as well as their responses to immunotherapy. For example, in MC38 tumors, approximately 20% of the TILs are CD8+ T cells compared with only 2.5% in B16-F10 tumors (FIGS. 12A-12B).

To test the activity of Compound 1 in a less immunogenic tumor model, mice were injected subcutaneously with B16-F10 melanoma cells. Tumors were allowed to grow to an average volume of 100 mm³ before mice were randomized to receive either PBS or various doses of WTX-124, using the same dosing schedule as before. Compound 1 tumor-infiltrating tetramer-positive manner (FIG. 6A) while anti-PD-1 treatment alone was ineffective in this model (FIG. 11). Interestingly, combining the lower dose of Compound 1 with PD-1 blockade demonstrated combinatorial activity. However, no additional benefit of PD-1 blockade was seen with a higher dose of Compound 1.

To further explore the mechanism of tumor growth inhibition, total tumor RNA was extracted from mice treated with Compound 1, 24 hours after the second dose, and analyzed using the NanoString nCounter® PanCancer Mouse Immune Profiling Panel. Compound 1 treatment resulted in a large transcriptional shift, with 184/770 examined transcripts statistically different after Compound 1 treatment (FIG. 6B). Interestingly, immune profiling by flow cytometry of the B16-F10 TILs did not reveal the large increase in immune cells observed in the MC38 tumors after Compound 1 treatment, which may explain the differences in anti-tumor efficacy between these two models at this dose (data not shown). However, Compound 1 treatment did induce proliferation and granzyme B production by tumor-infiltrating tetramer-positive CD8+ T cells (FIGS. 6C-6D) and NK cells (FIGS. 6E-6F). Furthermore, Compound 1 was more effective than an equimolar amount of recombinant hIL-2 at inducing CD25 expression and proliferation by tumor infiltrating NK cells, CD4+ NonTregs, total CD8+ T cells and tetramer+CD8+ T cells, likely due to the enhanced PK profile of the IL-2 prodrug compared to the free cytokine. Lastly, just as in the MC38 model, Compound 1 treatment of B16-F10 tumor-bearing mice resulted in a small subset of tumor-infiltrating FoxP3+ Tregs producing inflammatory cytokines such as TNF and IFNγ (FIGS. 6G-611), suggesting that Compound 1 treatment induces Treg fragility in this model as well. Together, these data demonstrate that Compound 1 treatment can induce TIL activation and inhibition of tumor growth in less inflamed, colder tumor models.

### Compound 1 was Stable in Human Serum and is Processed by Human Tumors

Human tumor samples are heterogeneous in nature and display different degrees of protease dysregulation and expression (15). Therefore, a screen was performed to identify potential INDUKINE™ molecule linkers based on stability in systemic circulation and processing by the majority of tumor types. The basis of this screen was a protease agnostic approach, where linkers cleaved specifically by primary human tumor samples were selected rather using a linker based on a specific target protease. This resulted in the selection of the linker sequence separating the different domains of Compound 1.

As a test of Compound 1 peripheral stability, the protein was incubated with human serum from healthy donors (n=3) for up to 72 hours before processing and measured by western blot. In agreement with the murine plasma experiments, Compound 1 was not processed by human serum in any of the tested donors (FIG. 7A). To test whether Compound 1 was processed by tumor samples, an ex vivo processing assay was developed. Briefly, dissociated human tumor samples were incubated with Compound 1 or control proteins for 48 hours before the resultant IL-2 activity was measured. Using Compound 1-NC as a proxy for the baseline activity of the intact Compound 1 prodrug, and pre-cut Compound las representative of a fully activated molecule, the activity induced by incubation of Compound 1 with the dissociated human tumor samples was normalized to a range of 0-100% activation. In some instances, the primary human tumor samples also contained viable TILs, which had the capacity to consume some of the free IL-2 in the pre-cut Compound 1 positive control group. This resulted in the activity of the positive control being artificially depressed in certain samples, which allows for the possibility of some samples recording over 100% activity compared to the positive control. Despite this issue, this assay is sufficient to be used as a binary analysis of whether or not Compound 1 is being processed by primary human tumor samples.

To examine how well Compound 1 was processed by various tissue samples, healthy primary human cells (n=13) and primary human tumor samples (n=97) were examined for the capacity to cleave Compound 1. The healthy primary cells were derived from various tissues, and the tumor samples covered a wide range of tumor types and stages. Importantly, exposure of Compound 1 to the healthy primary cells did not result in any evidence of cleavage, once again suggesting that this protein will be stable in the periphery in patients (FIG. 7B). In contrast, the majority of the tumor samples tested were able to process Compound 1. These data suggest that Compound 1 is stable when exposed to human serum or healthy primary cells but is efficiently activated by most human tumor samples, supporting its further development as a new immunotherapy for patients with cancer.

### 1.3 Discussion

High-dose IL-2 therapy was initially approved for patients with metastatic renal cell carcinoma in 1992 and for patients with advanced melanoma in 1998 (4). Before the advent of the modern field of immuno-oncology, high-dose IL-2 stood out as a treatment associated with complete responses, albeit in a minority of patients. However, the anti-tumor potential of proinflammatory cytokines, like IL-2, has been hindered by the serious toxicities linked to their systemic delivery and the engagement of target cells outside the tumor microenvironment (4). In the particular case of IL-2, several pharmaceutical and biotechnology companies have tried to minimize this problem by creating less-active forms of IL-2 (known as non-alpha molecules) that avoid activation of the IL-2 high-affinity receptor (7-10). Unfortunately, these molecular variants will still systemically activate cells carrying the medium-affinity receptor (CD122/CD132 subunits), which is responsible for the signal transduction of the cytokine, and they encounter similar toxicity problems as fully active IL-2 treatment at the doses required to see efficacy in preclinical models. Indeed, the non-α approach to IL-2 therapy may end up simply shifting the therapeutic window rather than improving it. Furthermore, newly activated CD8+ T cells upregulate CD25 to form the high-affinity receptor, which is required for their sustained expansion in the presence of antigens. For example, in a publication using a viral infection model, CD8+ T cells lacking CD25 failed to expand in infected tissues, despite expression of the medium-affinity receptor (22).

The design of inducible IL-2 addresses the challenges associated with rhIL-2 therapy. Inducible IL-2 contains a native IL-2 to realize the full pharmacological potential of this cytokine in driving anti-tumor immunity. The molecule is engineered as a prodrug to minimize the systemic toxicity and is conditionally activated to release IL-2 selectively in the tumor microenvironment. The activity of Compound 1 was highly inducible in vitro in human reporter cell assay systems as well as in human and mouse primary cells. Likewise, Compound 1 was efficacious in mouse syngeneic models and this efficacy was dependent on the tumor-specific processing. The half-life extension domain provides the opportunity for better drug exposure with less frequent dosing compared with the traditional dosing schedule for high-dose IL-2 therapy (proleukin). For example, although complete responses could be reliably generated in the MC38 mouse model by dosing twice a week, complete responses in 100% of the mice could also be achieved with doses as infrequent as once every 2 weeks, with slightly higher amounts of the prodrug. Furthermore, the peripheral inactivation provided by the IL-2 inactivation domain allowed for the safe administration of this IL-2 prodrug to mice at doses>20-fold higher than the dose required for potent efficacy but without obvious toxicity. Between the increased efficacy and decreased toxicity, Compound 1 has a significantly wider therapeutic window than previously described for high-dose IL-2.

The data reported in this publication demonstrate that Compound 1 efficacy is driven by the expansion and activation of effector cells in the tumor (both T cells and NK cells), which can produce effector cytokines such as TNF, granzyme B and IFN-γ. Indeed, activation of the tumor-infiltrating immune cells was sufficient to generate potent anti-tumor responses. Additionally, Compound 1 treatment increased the frequency of tumor-infiltrating polyfunctional CD8+ T cells, which are associated with greater cytolytic activity in viral models (18,19). One of the concerns expressed by proponents of non-α IL-2 therapies is that CD25 is highly expressed on Tregs, and therefore Treg expansion will inhibit anti-tumor immunity generated in response to wild-type IL-2. Although Compound 1 treatment did result in a slight Treg expansion, the expansion of CD8+ T cells far outpaced that of the Tregs, resulting in a favorable CD8/Treg ratio after treatment. Furthermore, WTX-124 treatment significantly increased the expression of IFN-γ by effector cells in a tumor-specific manner. IFN-γ is a fundamental effector cytokine that drives anti-tumor efficacy by amplifying the cellular immune component of the response and skewing CD4+ T cells towards a TH1 phenotype. Also, more recently, it has been shown that IFN-γ directs the mechanistic fragility of Tregs (20). This phenomenon was observed upon treatment with Compound 1, as the intratumoral Tregs began to produce cytokines traditionally associated with T effector cells, and it may contribute to the overall efficacy of Compound 1.

An important feature of Compound 1 is the selective processing of the prodrug in the tumor, allowing for systemic delivery, good exposure, and activation of the prodrug to release fully active IL-2 in the tumor microenvironment. Indeed, Compound 1 was highly stable while in circulation as shown in mice and in non-human primates (data not shown), as well as when WTX-124 was exposed to healthy primary human cells or plasma. In contrast, Compound 1 was reliably processed by primary human dissociated tumor samples from a wide variety of different cancer types, demonstrating the potential for systemically administered Compound 1 to selectively deliver IL-2 to the site of the disease and positively contribute to the development of an effective immune response. The clinical benefits and safety of Compound 1 treatment will be examined in the upcoming Phase I trial, subject to FDA clearance, testing Compound 1 either alone or in combination with the anti-PD-1 therapy pembrolizumab.

In summary, this work presents the design features and mechanistic characteristics of Compound 1, a novel, conditionally activated IL-2 prodrug that provides tumor-selective delivery of full potency IL-2 to activate tumor-specific immune cell populations.

### 1.4 References

1. Wei S C, Duffy C R, Allison J P. Fundamental mechanisms of immune checkpoint blockade therapy. Cancer Discov 2018; 8:1069-86.
2. Ribas A, Wolchok J D. Cancer immunotherapy using checkpoint blockade. Science 2018; 359:1350-5.
3. Pardoll D M. The blockade of immune checkpoints in cancer immunotherapy. Nat Rev Cancer 2012; 12:252-64.
4. Waldmann T A. Cytokines in cancer immunotherapy. Cold Spring Harb Perspect Biol 2018; 10.
5. Mitra S, Leonard W J. Biology of IL-2 and its therapeutic modulation: Mechanisms and strategies. J Leukoc Biol 2018; 103:643-55.
6. Malek T R. The biology of interleukin-2. Annu Rev Immunol 2008; 26:453-79.
7. Sharma M, Khong H, Fa'ak F, Bentebibel S E, Janssen L M E, Chesson B C, et al. Bempegaldesleukin selectively depletes intratumoral Tregs and potentiates T cell-mediated cancer therapy. Nat Commun 2020; 11:661.
8. Charych D H, Hoch U, Langowski J L, Lee S R, Addepalli M K, Kirk P B, et al. NKTR-214, an engineered cytokine with biased IL2 receptor binding, increased tumor exposure, and marked efficacy in mouse tumor models. Clin Cancer Res 2016; 22:680-90.
9. Klein C, Waldhauer I, Nicolini V G, Freimoser-Grundschober A, Nayak T, Vugts D J, et al. Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines. Oncoimmunology 2017; 6:e1277306.
10. Lopes J E, Fisher J L, Flick H L, Wang C, Sun L, Ernstoff M S, et al. ALKS 4230: a novel engineered IL-2 fusion protein with an improved cellular selectivity profile for cancer immunotherapy. J Immunother Cancer 2020; 8:e000673.
11. Mitschke J, Burk U C, Reinheckel T. The role of proteases in epithelial-to-mesenchymal cell transitions in cancer. Cancer Metastasis Rev 2019; 38:431-44.
12. Dudani J S, Warren A D, Bhatia S N. Harnessing protease activity to improve cancer care. Annu Rev Cancer Biol 2018; 2:353-76.
13. Yost K E, Satpathy A T, Wells D K, Qi Y, Wang C, Kageyama R, et al. Clonal replacement of tumor-specific T cells following PD-1 blockade. Nat Med 2019; 25:1251-9.
14. Konrad M W, Hemstreet G, Hersh E M, Mansell P W, Mertelsmann R, Kolitz J E, et al. Pharmacokinetics of recombinant interleukin 2 in humans. Cancer Res 1990; 50:2009-17.
15. Sands H, Loveless S E. Biodistribution and pharmacokinetics of recombinant, human 125I-interleukin-2 in mice. Int J mmunopharmacol 1989; 11:411-6.
16. Atkins M B, Lotze M T, Dutcher J P, Fisher R I, Weiss G, Margolin K, et al. High-dose recombinant interleukin 2 therapy for patients with metastatic melanoma: analysis of 270 patients treated between 1985 and 1993. J Clin Oncol 1999; 17:2105.
17. Ye X, Waite J C, Dhanik A, Gupta N, Zhong M, Adler C, et al. Endogenous retroviral proteins provide an immunodominant but not requisite antigen in a murine immunotherapy tumor model. Oncoimmunology 2020; 9:1758602.
18. Wherry E J, Blattman J N, Murali-Krishna K, Van Der Most R, Ahmed R. Viral persistence alters CD8 T-cell immunodominance and tissue distribution and results in distinct stages of functional impairment. J Virol 2003; 77:4911-27.
19. Imai N, Tawara I, Yamane M, Muraoka D, Shiku H, Ikeda H. CD4(+) T cells support polyfunctionality of cytotoxic CD8(+) T cells with memory potential in immunological control of tumor. Cancer Sci 2020; 111:1958-68.
20. Overacre-Delgoffe A E, Chikina M, Dadey R E, Yano H, Brunazzi E A, Shayan G, et al. Interferon-γ drives Treg fragility to promote anti-tumor immunity. Cell 2017; 169:1130-41. e11.
21. Matloubian M, Lo C G, Cinamon G, Lesneski M J, Xu Y, Brinkmann V, et al. Lymphocyte egress from thymus and peripheral lymphoid organs is dependent on S1P receptor 1. Nature 2004; 427:355-60.
22. D'Souza W N, Lefrançois L. IL-2 is not required for the initiation of CD8 T cell cycling but sustains expansion. J Immunol 2003; 171:5727-35.

1. A method for treating cancer, comprising administering to a subject in need thereof and effective amount of an inducible interleukin-2 (IL-2) prodrug, wherein the inducible IL-2 prodrug is administered systemically, is activated by cleavage by a protease that has higher activity in the tumor microenvironment than in other locations, and results in at least about 40-fold more cleavage of the inducible IL-2 prodrug in the tumor microenvironment compared with the circulation.

2. The method of claim 1, wherein at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 93-fold, at least about 95-fold, or at least about 100-fold more cleavage of the inducible IL-2 prodrug in the tumor microenvironment compared with the circulation.

3. The method of claim 1, wherein the administration results in an amount of inducible IL-2 prodrug in the plasma that is at least about 5-fold greater than the amount of inducible IL-2 prodrug in the tumor.

4. The method of claim 3, wherein the among of inducible IL-2 prodrug in the plasma is at least about 5-fold, at least about 10-fold, at least about 15-fold, at least about 18-fold, at least about 20-fold, or at least about 25-fold greater than the amount of inducible IL-2 prodrug in the tumor.

5. The method of claim 4, wherein the method results in a significant increase in the tumor reactive CD8+/Treg ratio.

6. A method for inducing immunological memory to a tumor, comprising administering to a subject in need thereof and effective amount of an inducible interleukin-2 (IL-2) prodrug, wherein the inducible IL-2 prodrug is administered systemically, is activated by cleavage by a protease that has higher activity in the tumor microenvironment than in other locations.

7. The method of claim 6, wherein at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 93-fold, at least about 95-fold, or at least about 100-fold more cleavage of the inducible IL-2 prodrug in the tumor microenvironment compared with the circulation.

8. The method of claim 6, wherein the administration results in an amount of inducible IL-2 prodrug in the plasma that is at least about 5-fold greater than the amount of inducible IL-2 prodrug in the tumor.

9. The method of claim 8, wherein the amount of inducible IL-2 prodrug in the plasma is at least about 5-fold, at least about 10-fold, at least about 15-fold, at least about 18-fold, at least about 20-fold, or at least about 25-fold greater than the amount of inducible IL-2 prodrug in the tumor.

10. The method of claim 6, wherein the immunological memory is characterized by tumor reactive CD8+ cells with a memory phenotype (e.g., CD8+CD44^{hi}CD62^{low})

11. The method of claim 6, wherein the immunological memory is characterized by tumor reactive CD8+ cells that produce TNF and/or IFNgamma upon restimulation.

12. The method of claim 6, wherein the immunological memory is characterized by polyfunctional tumor reactive CD8+ cells that produce TNF and IFNgamma upon restimulation.

13. The method of claim 6, wherein the tumor reactive CD8+ cells further produce granzyme B upon restimulation.

14. A method for selectively activating effector CD8+ T cells in the tumor microenvironment or tumor infiltrating lymphocytes, comprising administering to a subject in need thereof and effective amount of an inducible interleukin-2 (IL-2) prodrug, wherein the inducible IL-2 prodrug is administered systemically, is activated by cleavage by a protease that has higher activity in the tumor microenvironment than in other locations, and results a significantly higher frequency of CD8+ T cells that produce TNF and/or IFNgamma within the tumor in comparison to peripheral tissue.

15. (canceled)

16. The method of claim 14, wherein the administration results in at least about 40-fold more cleavage of the inducible IL-2 prodrug in the tumor microenvironment compared with the circulation.

17. The method of claim 16, wherein at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 93-fold, at least about 95-fold, or at least about 100-fold more cleavage of the inducible IL-2 prodrug in the tumor microenvironment compared with the circulation.

18. (canceled)

19. The method of claim 14, wherein the among of inducible IL-2 prodrug in the plasma is at least about 5-fold, at least about 10-fold, at least about 15-fold, at least about 18-fold, at least about 20-fold, or at least about 25-fold greater than the amount of inducible IL-2 prodrug in the tumor.

20. (canceled)

21. The method of claim 1, wherein the inducible IL-2 prodrug is Compound 1, Compound 2, Compound 3, Compound 4 or an amino acid sequence variant of any of the foregoing.

22. The method of any one of the preceding claims, wherein the inducible IL-2 prodrug is administered about twice a week or less frequently.

23-24. (canceled)
